(19) United States
(12) Patent Application Publication
(10) Pub. No.: US 2009/0269313 A1
(43) Pub. Date: Oct. 29, 2009

(54) ENCAPSULATION SYSTEM

(75) Inventor: Jerry L. Nadler, Charlottesville, VA (US)

Correspondence Address: NIXON & VANDERHYE, PC, 901 NORTH GLEBE ROAD, 11TH FLOOR, ARLINGTON, VA, 22203, US

(73) Assignee: DIAKINE THERAPEUTICS, INC., Charlottesville, VA (US)

(21) Appl. No.: 12/373,870

(22) PCT Filed: Jul. 19, 2007
(86) PCT No.: PCT/US07/73893

§ 371 Date: Jan. 14, 2009

(51) Int. Cl. A61K 35/12; A61P 3/10

(52) U.S. Cl. 424/93.7

(57) ABSTRACT

An encapsulation system for use in the treatment of diabetes (Types 1 or 2, and LADA) are provided. The system has (1) a delivery vehicle comprising a selectively permeable membrane that allows passage of glucose, insulin and other nutrients through the membrane, but prevents large molecules such as antibodies or inflammatory cells from passing through the membrane; (2) a population of islet cells or insulin producing cells encapsulated by said membrane; and (3) a biological response modifier that may be in contact with the membrane or encapsulated by the membrane. Generally, the biological response modifier is a compound, including resolved enantiomers, diastereomers, tautomers, salts and solvates thereof, having the following formula:

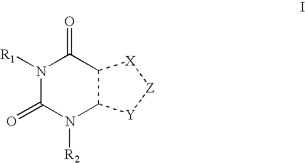

wherein:
X, Y and Z are independently selected from a member of the group consisting of C(R₃), N, N(R₃) and S;
R₁ is selected from a member of the group consisting of hydrogen, methyl, C₍₅₋₉₎alkyl, C₍₅₋₉₎alkenyl, C₍₅₋₉₎alkynyl, C₍₅₋₉₎hydroxyalkyl, C₍₃₋₈₎alkoxyl, C₍₅₋₉₎alkoxyalkyl, the R₁ being optionally substituted;
R₂ and R₃ are independently selected from a member of the group consisting of hydrogen, halo, oxo, C₍₁₋₂₀₎alkyl, C₍₁₋₂₀₎hydroxyalkyl, C₍₁₋₂₀₎thioalkyl, C₍₁₋₂₀₎alkylamino, C₍₁₋₂₀₎alkylaminoalkyl, C₍₁₋₂₀₎aminoalkyl, C₍₁₋₂₀₎aminoalkoxyalkenyl, C₍₁₋₂₀₎aminoalkoxyalkynyl, C₍₁₋₂₀₎diaminoalkyl, C₍₁₋₂₀₎triaminoalkyl, C₍₁₋₂₀₎tetraaminoalkyl, C₍₅₋₁₅₎aminotrialkoxyamino, C₍₁₋₂₀₎alkylamido, C₍₁₋₂₀₎alkylamidoalkyl, C₍₁₋₂₀₎amidoalkyl, C₍₁₋₂₀₎acetamidoalkyl, C₍₁₋₂₀₎alkenyl, C₍₁₋₂₀₎alkynyl, C₍₃₋₈₎alkoxyl, C₍₁₋₁₁₎alkoxyalkyl, and C₍₁₋₂₀₎dialkoxyalkyl.

### FIELD OF THE INVENTION

Transplantation of insulin producing cells to treat diabetes (Types 1 or 2 and Latent Autoimmune Diabetes in Adults (“LADA”)) is limited because transplanted cells are destroyed quickly by the recipient's immune system. To overcome this limitation, it is desirable that insulin-producing cells be enclosed in a semi-permeable membrane or device that would protect cells from immune attack, while allowing the influx of molecules important for cell function/survival and efflux of the desired cellular products.

### BACKGROUND OF THE INVENTION

Among the major obstacles in research directed to pancreatic islet transplantation for the treatment of diabetes is an inability to induce permissive acceptance of xenograft tissue transplants in the host mammal. Current methods of transplantation must suppress immune response by the host mammal that may lead to rejection of the transplanted cells and loss of islet function. Many transplantation approaches require the host to take general immunosuppressive agents to prevent a host immune response from destroying the transplanted tissue. However, such immunosuppressive agents are undesirable because they reduce the immune response of the host generally, and thus can lead to poor health. Thus, there is also a need in the art for a simple, non-invasive method of introducing a transplant into a host without requiring general immunosuppressive agents.

The principle of immunoisolation or immunoprotection of cells for transplantation overcomes two main obstacles: 1) cell transplantation without the need for immunosuppression and its accompanying side effects, and 2) transplantation of cells from non-human species (xenograft) to overcome the limited supply of donor cells (allografts) for such diseases as diabetes. Many diseases may be treated best by the regulated release of a cellular product (hormone, protein, neurotransmitter, etc.). Thus, a variety of cell types are candidates for transplantation of immunoisolated cells, including pancreatic islets (human or xeno), engineered beta-cells, stem cells, hepatocytes, neurons, parathyroid cells, etc. To combat rejection, immunosuppressive drugs have been used, but such immunosuppressive therapy impairs the body's immunological defenses and carries significant side effects and risks in itself.

It is well known that spheres may be prepared very readily, for example from sodium alginate, by using the property of alginate solutions to gel in the presence of cations such as, for example, calcium ions. The material to be encapsulated in the spheres is first dispersed in the aqueous alginate solution. This solution is added dropwise to an aqueous solution of a calcium salt. There is immediate gelation, which produces spheres of gelled alginate. The surface of the spheres may then be stabilized by immersion in a solution of a polycationic polymer such as poly-L-lysine or polyethyleneimine (Lim, F., U.S. Pat. No. 4,352,883). A membrane forms at the periphery, resulting from ionic association between the alginate and the polycation. This membrane allows small molecules to pass through while retaining large molecules and cells. It is then possible to liquefy the inner gel by immersing the alginate spheres, stabilized by the polycationic polymer, in a citrate solution, so as to chelate the calcium in the spheres (Lim, F., U.S. Pat. No. 4,352,883). The material incorporated then remains contained within the membrane.

Approximately one percent of the volume of the human pancreas is made up of islets of Langerhans (hereinafter “islets” or “pancreatic islets”), which are scattered throughout the exocrine pancreas. Each islet comprises insulin producing beta-cells as well as glucagon containing alpha cells, somatostatin secreting delta cells, and pancreatic polypeptide containing cells (PP-cells). The majority of islet cells are insulin-secreting beta-cells. Approaches to containing and protecting transplanted islet cells have been proposed, including the use of extravascular diffusion chambers, intravascular diffusion chambers, intravascular ultrafiltration chambers, macroencapsulation and micro-encapsulation. The goal of pancreatic islet transplantation is to achieve normal glycemic levels in a treated diabetic subject for some extended period of time. Compositions and methods of treating isolated pancreatic cells, or of treating encapsulated pancreatic cells, to enhance glucose-stimulated insulin production by the capsules and to provide durable capsules capable of glucose-stimulated insulin production, are therefore desirable.

Transplantation of pancreatic islets for the treatment of type 1 diabetes allows for physiologic glycemic control and insulin-independence when sufficient islets are implanted via the portal vein into the liver. Intrahepatic islet implantation requires specific infrastructure and expertise, and risks inherent to the procedure include bleeding, thrombosis, and elevation of portal pressure. Additionally, the relatively higher drug metabolite concentrations in the liver may contribute to the delayed loss of graft function of recent clinical trials. Identification of alternative implantation sites using biocompatible devices may be of assistance improving graft outcome. A workable bioartificial pancreas would be easy to implant, biopsy, and retrieve, while allowing for sustained graft function. The subcutaneous (SC) site for the bioartificial device may also require a minimally invasive procedure performed under local anesthesia.

One aspect of the present invention is directed to the use of bioartificial pancreatic constructs to immunoisolate pancreatic islets or insulin secreting cells to reverse established diabetes. The interest in this approach stems from the dire shortage of human pancreatic donors for pancreatic islets and the potential to reverse diabetes without the need for immunosuppressive drugs. In order to protect the viability of transplanted islet cells, devices or microcapsules have been developed to contain xenografts and thus allow islets from porcine or primate species to be used to reverse diabetes. It would also be possible to use this approach for allogenic transplants or cell lines that have been genetically engineered to release insulin in a glucose regulated fashion. The principle of these approaches is to separate the insulin delivery source form the immune system of the host by a selectively permeable membrane. These systems allow glucose and other nutrients in and insulin to be secreted in response to ambient glucose levels. However, large molecules such as antibodies or inflammatory cells cannot enter.

Encapsulation of insulin producing cells has shown some success in reversing chemically-induced diabetes in rodents and in a small scale human clinical trial. Most cell encapsulation currently utilizes modifications of the procedure originated by Lim and Sun in which the encapsulant is suspended in a polyanionic aqueous solution and extruded by an air jet/syringe pump droplet generator into calcium ions. The method of microencapsulation described by Lim and Sun involves forming gelled alginate droplets around isolated islet cells, and then adding coats of poly-L-lysine and additional alginate. Poly(L-lysine), which is a cationic macromolecule, is mixed with the hardened polyanionic gel, and a membrane is formed at the interface as a result of the ionic interaction. See e.g., U.S. Pat. Nos. 4,352,883, 4,352,883 and 4,806,355, the disclosures of which are expressly incorporated herein by reference. The inner gelled core of the microcapsule is then liquefied by chelation. However, chelation of the core affects the structural support of the capsules and may adversely affect durability. The success of microencapsulated islet cell transplantation in treating diabetes depends on the ability of the microcapsules to provide sufficient amounts of insulin in response to glucose stimulation, over an extended period of time, to achieve adequate glycemic control. In principle these “capsules” or “devices” could work. However, in the setting of Type 1 diabetes or xenografts, undesirable inflammatory cytokines are often involved (e.g., interleukins, IL1, IL12, and IL18, tumor necrosis factor, or Interferon gamma), which enter the capsules or devices and lead to reduced function or apoptotic cell death of the islet tissue or insulin secreting cells. Since insulin and the cytokines are of similar size, it is difficult to prepare a semipermeable membrane that would allow insulin to be released and simultaneously prevent cytokines from entering the device. Therefore, an agent capable of protecting encapsulated islets or insulin secreting cells form cytokine damage would have clinical value because such an agent would facilitate the function and longevity of transplanted encapsulated cells or tissues that secrete/produce glucose-stimulated insulin without the chronic use of immune suppressant drugs.

### SUMMARY OF THE INVENTION

According to principles of the present invention, the use of BRMs to prevent immune damage and enhance the function of encapsulated islets or insulin producing cells is provided herein. Encapsulation involves the surrounding of insulin producing cells with a biocompatible biopolymer prior to implantation, which reduces the host's immune response to the implanted material. Biological Response Modifiers (“BRMs”) can enhance encapsulation techniques by reducing inflammatory cytokine-induced damage to pancreatic islet cells and isolated beta-cells. BRMs also enhance glucose induced insulin secretion thus improving the function of these insulin secreting cells. Preferred BRM compounds are described below. In a preferred embodiment, the BRMs can be delivered to a subject systemically by intravenous means or by orally administration routes.

Preferrably, BRMs are incorporated into a semipermeable membrane or encapsulation system to protect locally insulin producing cells from inflammatory cytokines. For example, pancreatic islet cells are treated with a BRM alone or in combination with another compound (e.g., an antioxidant, a beta-cell growth, differentiating or neogenesis factor, an anti-endotoxin or an antibiotic) in a medium for culturing the cells before encapsulation; in a medium for cryopreserving the cells by freezing followed by thawing and encapsulation; in a medium for culturing the cells after encapsulation; or in a medium for culturing the cells before encapsulation. The inventive methods of treating cells and microcapsules may be combined, for example, by culturing isolated cells prior to microencapsulation and then culturing the resulting microcapsules.

### DESCRIPTION OF PREFERRED EMBODIMENTS

All patents, patent applications and literatures cited or referenced in this description are incorporated herein by reference in their entirety. In the case of inconsistencies, the present disclosure, including definitions, will control.

### DEFINITIONS

As used herein, materials that are intended to come into contact with biological fluids or tissues (such as by implantation or transplantation into a subject) are termed “biomaterials”. It is desirable that biomaterials induce minimal reactions between the material and the physiological environment. Biomaterials are considered “biocompatible” if, after being placed in the physiological environment, there is minimal inflammatory reaction, no evidence of anaphylactic reaction, and minimal cellular growth on the biomaterial surface. Upon implantation in a host mammal, a biocompatible microcapsule does not elicit a host response sufficient to detrimentally affect the function of the microcapsule; such host responses include formation of fibrotic structures on or around the microcapsule, immunological rejection of the microcapsule, or release of toxic or pyrogenic compounds from the microcapsule into the surrounding host tissue.

The term “encapsulate” as used herein refers to the containment of a cell or cells within a capsule delineated by a physical barrier (i.e., a barrier that reduces or controls the permeability of the capsule).

The term “microcapsule” or “microsphere” as used herein refers to a structure containing a core of biological substance (such as cells) in an aqueous medium, surrounded by a semi-permeable membrane, and having a diameter of no more than 2 mm. Preferably, microspheres are from about 3 μm to about 2 mm in diameter. More preferably, microcapsules range from about 50 μm to about 1,000 μm in diameter, or from about 300 μm to about 500 μm in diameter. Depending on the method of microencapsulation used, it will be apparent that the microcapsule wall or membrane may also contain some cells therein.

As used herein, the term “culture” refers to the maintenance or growth of cells on or in a suitable nutrient medium, after removal of the cells from the body. Suitable nutrient culture media are readily available commercially, and will be apparent to those skilled in art given the cell type to be cultured.

The term “cells” as used herein refers to cells in various forms, including but not limited to cells retained in tissue, cell clusters (such as pancreatic islets or portions thereof), and individually isolated cells.

A first aspect of the present invention is a method of treating isolated pancreatic islet cells by first culturing the cells in a medium containing at least one BRM or a combination of a BRM and an antioxidant, a beta-cell growth, differentiating or neogenesis factor, an anti-endotoxin, or an antibiotic. The cells are then microencapsulated in a biocompatible microcapsule that may, for example, contain a hydrogel core and a semipermeable outer membrane, to provide a microcapsule containing living cells therein.

A further aspect of the present invention is a method of treating isolated pancreatic islet cells, by first cryopreserving the cells in a cryopreservation medium containing at least one BRM or a combination of a BRM and a beta-cell growth, differentiating or neogenesis factor, an antioxidant, an anti-endotoxin, or an antibiotic; then thawing the cells and encapsulating the cells in a biocompatible microcapsule having, for example, a hydrogel core and a semipermeable outer membrane.

A still further aspect of the present invention is a method of treating biocompatible microcapsules containing living cells, where the microcapsule contains, for example, a hydrogel core and a semipermeable outer membrane. The microcapsules are cultured in a medium containing at least one BRM or a combination of a BRM and a beta-cell growth, differentiating or neogenesis factor, an antioxidant, an anti-endotoxin, or an antibiotic.

A further aspect of the present invention is a method of preparing microencapsulated cells by first culturing the cells in a cell culture medium containing at least one BRM or a combination of a BRM and a beta-cell growth, differentiating or neogenesis factor, an antioxidant, an anti-endotoxin, or an antibiotic. The cells are then encapsulated in a biocompatible microcapsule having, for example, a hydrogel core and a semipermeable outer membrane, where the living cells are present in the core. The microcapsules are then cultured in a medium containing at least one BRM (or a combination of a BRM and an antioxidant, an anti-endotoxin, and an antibiotic.

Preferrably, isolated insulin producing cells (e.g., pancreatic islet cells (human or xeno)) are cultured for a sufficient period of time (e.g., from about 12 to as long as about 36 hours) in a medium containing a BRM, cryopreserved by freezing in a medium containing the BRM or combination of compounds, thawed and microencapsulated.

In a preferred embodiment the composition is encapsulated by the semipermeable membrane. For example Lisofylline can be solubilized and the capsule can be formed in the Lisofylline solution. Alternatively, the BRM may be linked or embedded in the polymer matrix comprising the semipermeable membrane. The BRM can be linked to the polymer matrix (e.g., via covalent bonds, ionic bonds, hydrogen bonds, aromatic bonds, metallic bonds, hydrophobic/hydrophilic interactions, etc.). In a preferred embodiment, BRMs are linked to reactive groups located on the polymers prior to formation of the polymer matrix. In another preferred embodiment of the invention, a composition is provided comprising a selectively permeable membrane that allows passage of glucose, insulin and other nutrients through the membrane, but prevents large molecules such as antibodies or inflammatory cells from passing through the membrane and a BRM, wherein the BRM is linked to the polymer components of the membrane. This membrane can be formed as a capsule that encapsulates a viable population of islet cells or insulin producing/secreting cells. The BRMs described herein can also be used to enhance the function of insulin secreting cells that are resistant to interleukin/beta and gamma interferon (Diabetes 49:562-570, 2000) or genetically engineered cells that can release insulin.

In another preferred aspect, and as an alternative to intrahepatic islet transplantation, a biocompatible device (e.g., bio-artificial organ) may be employed. See e.g., Pileggi, Antonello, et al., “Reversal of Diabetes by Pancreatic Islet Transplantation into a Subcutaneous, Neovascularized Device.” Transplantation 81(9):1318-1324 (May 15, 2006). For example, the device may be approximately 2 centimeters long (about one inch) or longer with a diameter of approximately half-a-centimeter or more. The device may be made of any biocompatible material (e.g., a cylindrical stainless steel mesh) and comprise plastic (polytetrafluoroethylene) caps and a plug. The device (with the plug in place) may be implanted in the omental fat or under the skin prior to allow embedding by connective tissue and neovascularization (e.g., for approximately 40 days) whereby tissue and new blood vessels are allowed to proliferate around and inside the device. Then, the plug may be removed and replaced with islet cells or insulin secreting cells (as described herein) inside the pre-vascularized device optionally along with a BRM (as described herein) or a combination of a BRM and a beta-cell growth, differentiating or neogenesis factor, an antioxidant, an anti-endotoxin, or an antibiotic. The device may then be capped. Reversal of diabetes and glycemic control can be monitored after islet transplantation to observe restored euglycemia and sustained function long-term.

Beta-Cell Growth, Differentiating or Neogenesis Factors

Preferred factors/agents that could be used to induce pancreatic beta-cell or insulin producing cell growth differentiation and/or neogenesis include, but are not limited to, one or more members of the group consisting of:
glucagon-like peptide 1 (GLP-1);
long-acting, DPP-IV-resistant GLP-1 analogs thereof, including, without limitation, members of the group consisting of Exendin-4 (Ex-4), Exenatide (Byetta®, Amylin Pharmaceuticals), Exenatide LAR and related analogs disclosed in U.S. Pat. No. 5,424,286, U.S. Pat. No. 6,858,576, U.S. Pat. No. 6,872,700, U.S. Pat. No. 6,902,744, U.S. Pat. No. 6,956,026, U.S. Pat. No. 6,899,883 and U.S. Pat. No. 6,989,148 (the entire disclosures of which are incorporated herein by reference), Liraglutide (a.k.a., NN2211 or Arg(34)Lys(26)-(N-epsilon-(gamma-Glu(N-alpha-hexadecanoyl))-GLP-1 (7-37)) (Novo Nordisk), CJC-1131 (Conjuchem Inc.), Albugon (Human Genome Sciences), LY-548806 (Eli Lilly & Co), and the like;
inhibitors of GLP-1 degradation (a.k.a., DPP-IV inhibitors), which may be orally administered drugs that improve glycemic control by preventing DPP-IV degradation of GLP-1 and GIP and increasing incretin hormone levels to restore beta-cell mass or function, including, without limitation, members of the group consisting of Sitagliptin (a.k.a. MK-0431, Merck), Vildagliptin (a.k.a. LAF-237) and NVP DPP728 (both of Novartis), Saxagliptin (Bristol Myers Squibb), P32/98 (Probiodrug) and FE 999011 (a.k.a. [(2S)-1-([2′S]-2′-amino-3′,3′ dimethyl-butanoyl)-pyrrolidine-2-carbonitrile] developed by Ferring Research Institute), PHX1149 (Phenomix), and the like;
gastric inhibitory polypeptide (GIP) and analogs thereof (e.g., which are disclosed in U.S. Patent Publication No. 20050233969),
peptides such as gastrin and/or epidermal growth factor 1, including islet neogenesis therapy (Transition Therapeutics),
insulin like growth factor 1 or 2;
Parathyroid hormone related peptide (PTHrP) and
Hepatocyte growth factor or islet neogenesis associated protein (INGAP).

Other preferred agents include, without limitation, providing one or any combination of transcription factors shown to be important for insulin gene transcription or β-cell growth or development, including, without limitation, members of the group consisting of Neurogen 3, PDX-1, NKX6.1 and the like.

Other preferred agents that induce pancreatic β-cell or insulin producing cell growth and/or differentiation include, but are not limited to, members of the group consisting of: histone deacetylose inhibitors (HDAC) such as NVP-LAQ824, TrichostatinA-0, hydroxamate, suberanihohydroxamic or cyclic tetrapeptides, apicidin and trapoxin as well as synthetic inhibitors, including CG1521 and others, scriptide and analogs. Other HDAC inhibitors include: oxamflatin, pyroxamide, propenamides, chlamydocin, diheteropeptin, WF-3136, Cyl-1 and Cyl-2, FR 901228, cyclic-hydroxamic-acid-containing peptides, MS-275, CI-994 and depudecin.

Still other examples of preferred agents that induce pancreatic β-cell or insulin producing cell growth and/or differentiation include, but are not limited to, amino-terminal extended forms of GLP-1 selected from the group consisting of: (a) glucagon-like peptide 1(7-37); (b) glucagon-like peptide 1(7-36) amide; and (c) an effective fragment or analog of (a) or (b) (each of which are described in U.S. Pat. No. 6,899,883 and U.S. Pat. No. 6,989,148).

Biological Response Modifiers

Preferred BRMs include, without limitation, members selected from the group consisting of:

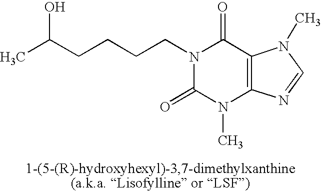

and related analogs such as

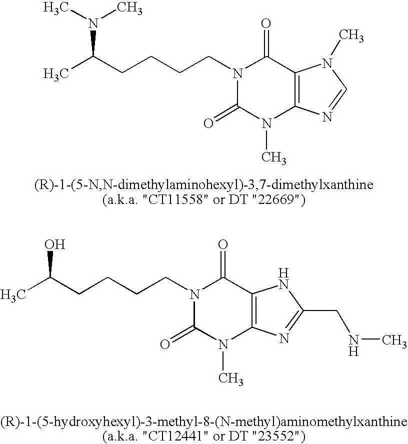

Without wishing to be bound by any theory of operation or mode of action, BRMs exhibit anti-inflammatory function by reducing inflammatory cytokine production or downstream effects (including, without limitation, IL-12, IL-23, IL-27, TNF-α, IFN-γ, IL-6 and IL-1β), selectively suppressing neutrophil and leukocyte adhesion and phagocytic activity, and decreasing neutrophil migration and degranulation during sepsis. More significantly, BRMs allows retention of beta-cell insulin secretory function after inflammatory cytokine insult and regulates immune cellular function to prevent autoimmunity. In addition, BRMs also exhibit the ability to ameliorate hemorrhage-induced tissue injury and to preserve tissue function during decreased blood flow or in poorly ventilated conditions. BRMs have been shown to reduce autoimmune reoccurrence in islet transplantation in NOD mice and protect human islets from inflammatory injury. All of these characteristics render the BRMs disclosed herein (e.g., LSF and the LSF analogs) capable of improving biological function and reducing autoimmune damage in insulin producing cells. LSF and its analogs disclosed herein represent a new class of immunomodulatory compounds that are capable of regulating cellular functions but retain host immune competence.

Further preferred BRMs include, without limitation, compounds, pharmaceutically acceptable derivatives (e.g., racemic mixtures, resolved enantiomers, diastereomers, tautomers, salts and solvates thereof) or prodrugs thereof, having the following Formula I:

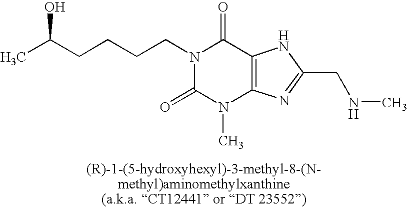

wherein:

the dashed lines, i.e., [symbol] in Formula I represent a single or double bond;

X, Y and Z are independently selected from a member of the group consisting of C(R3), N, N(R3) and S;

R1 is selected from a member of the group consisting of hydrogen, methyl, a substituted alkyl (as defined herein, which includes without limitation substituted C(5-9)alkyl), C(5-9)alkenyl, C(5-9)alkynyl, C(5-9)hydroxyalkyl, C(3-8)alkoxyl, C(5-9)alkoxyalkyl; and

R2 and R3 are independently selected from a member of the group consisting of hydrogen, halo, oxo, C(1-20)alkyl, C(1-20)hydroxyalkyl, C(1-20)thioalkyl, C(1-20)alkylamino, C(1-20)alkylaminoalkyl, C(1-20)aminoalkyl, C(1-20)aminoalkoxyalkenyl, C(1-20)aminoalkoxyalkynyl, C(1-20)diaminoalkyl, C(1-20)triaminoalkyl, C(1-20)tetraaminoalkyl, C(5-15)aminotrialkoxyamino, C(1-20)alkylamido, C(1-20)alkylamidoalkyl, C(1-20)amidoalkyl, C(1-20)acetamidoalkyl, C(1-20)alkenyl, C(1-20)alkynyl, C(3-8)alkoxyl, C(1-11)alkoxyalkyl, and C(1-20)dialkoxyalkyl.

R1 is optionally substituted with a member selected from the group consisting of N—OH, acylamino, cyano (e.g., NC—), cyanamido (e.g., NCNH—), cyanato (e.g., NCO—), sulfo, sulfonyl, sulfinyl, sulfhydryl (mercapto), sulfeno, sulfanilyl, sulfamyl, sulfamino, and phosphino, phosphinyl, phospho, phosphono and —NRaRb, wherein each of Ra and Rb may be the same or different and each is independently selected from the group consisting of hydrogen, optionally substituted alkyl, cycloalkyl, alkenyl, cycloalkenyl, alkynyl, aryl, heteroaryl and heterocyclic group.

Each R2 and R3 is optionally substituted with one or more members of the group consisting of hydroxyl, methyl, carboxyl, furyl, furfuryl, biotinyl, phenyl, naphthyl, amino group, amido group, carbamoyl group, cyano (e.g., NC—), cyanamido (e.g., NCNH—), cyanato (e.g., NCO—), sulfo, sulfonyl, sulfinyl, sulfhydryl (mercapto), sulfeno, sulfanilyl, sulfamyl, sulfamino, phosphino, phosphinyl, phospho, phosphono, N—OH, —Si(CH3)3 (a.k.a. SiMe3), C(1-3)alkyl, C(1-3)hydroxyalkyl, C(1-3)thioalkyl, C(1-3)alkylamino, benzyldihydrocinnamoyl group, benzoyldihydrocinnamido group, heterocyclic group and carbocyclic group.

The heterocyclic group or carbocyclic group is optionally substituted with one or more members of the group consisting of halo, hydroxyl, nitro (e.g., —NO2), SO2NH2, C(1-6)alkyl, C(1-6)haloalkyl, C(1-8)alkoxyl, C(1-11)alkoxyalkyl, C(1-6)alkylamino, and C(1-6)aminoalkyl.

Preferably, both X and Y are not N(R3) when Z is C(R3) and R3 is H or C(1-3)alkyl.

More preferably, R1 is not an ω-1 secondary alcohol substituted C(5-8) alkyl when both X and Y are N(R3), Z is C(R3) and R3 is H or C(1-3) alkyl.

In another preferred aspect of the present invention, R1 is an ω-1 secondary alcohol substituted C(5-8) alkyl when both X and Y are N(R3), Z is C(R3) and R3 is H or C(1-3) alkyl.

In a another aspect, more preferred LSF analog compounds include the following compounds, pharmaceutically acceptable derivatives (e.g., racemic mixtures, resolved enantiomers, diastereomers, tautomers, salts and solvates thereof) or prodrugs thereof, having the following Formula II:

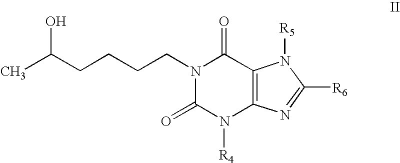

wherein R₄, R₅ and R₆ are independently selected from a member of the group consisting of hydrogen, halo, oxo, C₍₁₋₂₀₎alkyl, C₍₁₋₂₀₎hydroxyalkyl, C₍₁₋₂₀₎thioalkyl, C₍₁₋₂₀₎alkylamino, C₍₁₋₂₀₎alkylaminoalkyl, C₍₁₋₂₀₎aminoalkyl, C₍₁₋₂₀₎aminoalkoxyalkenyl, C₍₁₋₂₀₎aminoalkoxyalkynyl, C₍₁₋₂₀₎diaminoalkyl, C₍₁₋₂₀₎triaminoalkyl, C₍₁₋₂₀₎tetraaminoalkyl, C₍₃₋₁₅₎aminodialkoxyamino, C₍₅₋₁₅₎aminotrialkoxyamino, C₍₁₋₂₀₎alkylamido, C₍₁₋₂₀₎alkylamidoalkyl, C₍₁₋₂₀₎amidoalkyl, C₍₁₋₂₀₎acetamidoalkyl, C₍₁₋₂₀₎alkenyl, C₍₁₋₂₀₎alkynyl, C₍₃₋₈₎alkoxyl, C₍₁₋₁₁₎alkoxyalkyl, and C₍₁₋₂₀₎dialkoxyalkyl.

Each R4, R5 and R6 is optionally substituted with one or more members of the group consisting of hydroxyl, methyl, carboxyl, furyl, furfuryl, biotinyl, phenyl, naphthyl, amino group, amido group, carbamoyl group, cyano (e.g., NC—), cyanamido (e.g., NCNH—), cyanato (e.g., NCO—), sulfo, sulfonyl, sulfinyl, sulfhydryl (mercapto), sulfeno, sulfanilyl, sulfamyl, sulfamino, phosphino, phosphinyl, phospho, phosphono, N—OH, —Si(CH3)3, C(1-3)alkyl, C(1-3)hydroxyalkyl, C(1-3)thioalkyl, C(1-3)alkylamino, benzyldihydrocinnamoyl group, benzoyldihydrocinnamido group, heterocyclic group and carbocyclic group.

The heterocyclic group or carbocyclic group is optionally substituted with one or more members of the group consisting of halo, hydroxyl, nitro (e.g., —NO2), SO2NH2, C(1-6) alkyl, C(1-6)haloalkyl, C(1-8)alkoxyl, C(1-11)alkoxyalkyl, C(1-6)alkylamino, and C(1-6) aminoalkyl. In a preferred embodiment, each R4, R5 and R6 are not simultaneously methyl.

In a preferred embodiment, both R4 and R5 are not methyl when R6 is H.

In another preferred embodiment, R6 is not methyl when R4 is methylfuryl and R5 is H.

In a further preferred embodiment, R6 is not propyl or isopropyl when R4 is methyl and R5 is H.

In a still further preferred embodiment, R4 is not acetamidohexyl when R5 is methyl and R6 is H.

Preferred examples of R2, and R3 groups of Formula I and R4, R5 and R6 groups of Formula II include, without limitation, members selected from the group consisting of 1-adamantanemethyl, 1-phenylcyclopropyl, 1-phenylproply, 1-propenyl, 2-bromopropyl, 2-buten-2-yl, 2-butyl, 2-cyclohexylethyl, 2-cyclopentylethyl, 2-furyl, 2-hydroxyethyl, 2-hydroxystyryl, 2-methoxyethyl, 2-methoxystyryl, 2-methylbutyl, 2-methylcyclopropyl, 2-norboranemethyl, 2-phenylpropyl, 2-propenyl, 2-propyl, 2-thienyl, 2-trifluoromethylstyryl, 3,4,5-triethoxyphenyl, 3,4,5-trimethoxyphenyl, 3,4-dichlorobenzyl, 3,4-dichlorophenyl, 3,4-difluorophenyl, 3,4-difluorobenzyl, 3,4-dihydroxybenzyl, 3,4-dihydroxystyryl, 3,4-dimethoxybenzyl, 3,4-dimethoxyphenethyl, 3,4-dimethoxyphenyl, 3,4-dimethoxystyryl, 3,4-dimethylphenyl, 3,5-bis(trifluoromethyl)-benzyl, 3,5-dimethylphenyl, 3-bromo-4-methylphenyl, 3-bromobenzyl, 3-cyclohexylpropyl, 3-dimethylaminobutyl, 3-fluoro-4-methylphenyl, 3-fluorobenzyl, 3-hepten-3-yl, 3-hydroxy-n-butyl, 3-hydroxypropyl, 3-iodo-4-methylphenyl, 3-methoxy-4-methylphenyl, 3-methoxybenzyl, 3-methylbenzyl, 3-phenylpropyl, 3-trifluoromethylbenzyl, 4′-ethyl-4-biphenyl, 4-biphenyl, 4-bromobenzyl, 4-bromophenyl, 4-butylphenyl, 4-chloropentyl, 4-chlorostyryl, 4-ethoxybenzyl, 4-fluorobenzyl, 4-fluorophenyl, 4-hydroxyphenyl, 4-isobutylphenethyl, 4-isopropylphenyl, 4-methoxybenzyl, 4-methoxy-n-butyl, 4-methylbenzyl, 4-methylcyclohexanemethyl, 4-methylcyclohexyl, 4-phenylbenzyl, 4-t-butylcyclohexyl, 4-vinylphenyl, 5-hydroxyhexyl, alpha-methylstyryl, benzyl, cyclobutyl, cycloheptyl, cyclohexyl, cyclohexylmethyl, cyclopentyl, ethyl, hexyl, isobutyl, isopropyl, isovaleryl, m-anisyl, methyl, m-tolyl, n-butyl, n-propyl, p-anisyl, phenethyl, phenyl, propyl, p-tolyl, styryl, t-butyl, and the like.

Preferred R2, R3, R4, R5 and R6 groups include, without limitation, members selected from the group consisting of methyl, ethyl, oxo, isopropyl, n-propyl, isobutyl, n-butyl, t-butyl, 2-hydroxyethyl, 3-hydroxypropyl, 3-hydroxy-n-butyl, 2methoxyethyl, 4-methoxy-n-butyl, 5-hydroxyhexyl, 2-bromopropyl, 3-dimethylaminobutyl, 4-chloropentyl, methylamino, aminomethyl, methylphenyl, and the like.

In accordance with the present invention, the LSF compounds, LSF analogs, salts, solvates and prodrugs thereof, may exist in their tautomeric form (for example, as an amide or imino ether). All such tautomeric forms are contemplated herein as part of the present invention. Further, all stereoisomers (for example, geometric isomers, optical isomers and the like) of the present compounds (including those of the salts, solvates and prodrugs of the compounds as well as the salts and solvates of the prodrugs), such as those which may exist due to asymmetric carbons on various substituents, including enantiomeric forms (which may exist even in the absence of asymmetric carbons), rotameric forms, atropisomers, and diastereomeric forms, are contemplated within the scope of this invention, as are positional isomers (such as, for example, 4-pyridyl and 3-pyridyl). Individual stereoisomers of the compounds described herein as suitable for use in the present invention may, for example, be substantially free of other isomers, or may be admixed, for example, as racemates or with all other, or other selected, stereoisomers. The chiral centers of the present invention can have the S or R configuration as defined by the IUPAC 1974 Recommendations. The use of the terms “salt”, “solvate” “prodrug” and the like, is intended to equally apply to the salt, solvate and prodrug of enantiomers, stereoisomers, rotamers, tautomers, positional isomers, racemates or prodrugs of compounds disclosed herein.

In accordance with the principles of the present invention, the LSF analogs described herein may contain one or more asymmetrically substituted carbon atoms and, thus, may occur as racemates and racemic mixtures, single enantiomers, diastereomeric mixtures and individual diastereomers. Each stereogenic carbon may be of the R or S configuration. Many geometric isomers of olefins, C—N double bonds, and the like can also be present in the compounds described herein, and all such stable isomers are contemplated in the present invention. It is well known in the art how to prepare optically active forms, such as by resolution of racemic forms or by synthesis from optically active starting materials. All chiral, diastereomeric, racemic forms and all geometric forms of a structure are intended to be encompassed within the present invention unless a specific stereochemistry or isomer form is specifically indicated.

The compounds of the present invention may be modified by appending appropriate functionalites to enhance selective biological properties. Such modifications are known in the art and include, without limitation, those which increase penetration into a given biological compartment (e.g., blood, lymphatic system, central nervous system), increase oral or intravenous bioavailability, increase solubility to allow administration by injection, alter metabolism, alter rate of excretion, etc.

“Stereoisomer” or “Optical isomer” as used herein means a stable isomer that has at least one chiral atom or restricted rotation giving rise to perpendicular dissymmetric planes (e.g., certain biphenyls, allenes, and spiro compounds) and can rotate plane-polarized light. Because asymmetric centers and other chemical structure exist in the compounds described herein as suitable for use in the present invention which may give rise to stereoisomerism, the invention contemplates stereoisomers and mixtures thereof. The compounds described herein and their salts include asymmetric carbon atoms and may therefore exist as single stereoisomers, racemates, and as mixtures of enantiomers and diastereomers. Typically, such compounds will be prepared as a racemic mixture. If desired, however, such compounds can be prepared or isolated as pure stereoisomers, i.e., as individual enantiomers or diastereomers, or as stereoisomer-enriched mixtures. As discussed in more detail below, individual stereoisomers of compounds are prepared by synthesis from optically active starting materials containing the desired chiral centers or by preparation of mixtures of enantiomeric products followed by separation or resolution, such as conversion to a mixture of diastereomers followed by separation or recrystallization, chromatographic techniques, use of chiral resolving agents, or direct separation of the enantiomers on chiral chromatographic columns. Starting compounds of particular stereochemistry are either commercially available or are made by the methods described below and resolved by techniques well-known in the art.

“Enantiomers” as used herein means a pair of stereoisomers that are non-superimposable mirror images of each other.

“Diastereoisomers” or “Diastereomers” as used herein mean optical isomers which are not mirror images of each other.

“Racemic mixture” or “Racemate” as used herein means a mixture containing equal parts of individual enantiomers.

“Non-Racemic Mixture” as used herein means a mixture containing unequal parts of individual enantiomers.

“Stable compound”, as used herein, is a compound that is sufficiently robust to survive isolation to a useful degree of purity from a reaction mixture, and formulation into an efficacious therapeutic agent, i.e., possesses stability that is sufficient to allow manufacture and that maintains the integrity of the compound for a sufficient period of time to be useful for the purposes detailed herein (e.g., therapeutic or prophylactic administration to a mammal or for use in affinity chromatography applications). Typically, such compounds are stable at a temperature of 40° C. or less, in the absence of moisture or other chemically reactive conditions, for at least a week. “Metabolically stable compound” denotes a compound that remains bioavailable when orally ingested by a mammal.

“Substituted”, as used herein, whether express or implied and whether preceded by “optionally” or not, means that any one or more hydrogen on the designated atom (C, N, etc.) is replaced with a selection from the indicated group, provided that the designated atom's normal valency is not exceeded, and that the substitution results in a stable compound. For instance, when a CH2 is substituted by a keto substituent (═O), then 2 hydrogens on the atom are replaced. It should be noted that when a substituent is listed without indicating the atom via which such substituent is bonded, then such substituent may be bonded via any atom in such substituent. For example, when the substituent is piperazinyl, piperidinyl, or tetrazolyl, unless specified otherwise, said piperazinyl, piperidinyl, tetrazolyl group may be bonded to the rest of the compound of Formula I or II, as well as the R2, R3, R4, R5 and R6 groups substituted thereon, via any atom in such piperazinyl, piperidinyl, tetrazolyl group. Combinations of substituents and/or variables are permissible only if such combinations result in stable compounds. Further, when more than one position in a given structure may be substituted with a substituent selected from a specified group, the substituents may be either the same or different at every position. Typically, when a structure may be optionally substituted, 0-15 substitutions are preferred, 0-5 substitutions are more preferred, and 0-1 substitution is most preferred.

“Optional” or “optionally” as used herein means that the subsequently described event or circumstance may or may not occur, and that the description includes, without limitation, instances where said event or circumstance occurs and instances in which it does not. For example, optionally substituted alkyl means that alkyl may or may not be substituted by those groups enumerated in the definition of substituted alkyl.

“Acyl” as used herein denotes a radical provided by the residue after removal of hydroxyl from an organic acid. Examples of such acyl radicals include, without limitation, alkanoyl and aroyl radicals. Examples of such lower alkanoyl radicals include, without limitation, formyl, acetyl, propionyl, butyryl, isobutyryl, valeryl, isovaleryl, pivaloyl, hexanoyl, trifluoroacetyl.

“Acylamino” as used herein denotes an N-substituted amide, i.e., RC(O)—NH and RC(O)—NR′—. A non-limiting example is acetamido.

“Acyloxy” as used herein means 1 to about 4 carbon atoms. Preferred examples include, without limitation, alkanoyloxy, benzoyloxy and the like.

“Alkyl” or “lower alkyl” as used herein is intended to include both branched and straight-chain saturated aliphatic hydrocarbon radicals/groups having the specified number of carbon atoms. In particular, “alkyl” refers to a monoradical branched or unbranched saturated hydrocarbon chain, preferably having from 1 to 40 carbon atoms, more preferably 1 to 10 carbon atoms, even more preferably 1 to 6 carbon atoms, such as methyl, ethyl, n-propyl, isopropyl, n-butyl, secondary butyl, tert-butyl, n-hexyl, n-octyl, n-decyl, n-dodecyl, 2-ethyldodecyl, tetradecyl, and the like, unless otherwise indicated.

“Substituted alkyl” as used herein refers to an alkyl group as defined above having from 1 to 5 substituents selected, without limitation, from the group consisting of alkoxyl, substituted alkoxyl, cycloalkyl, substituted cycloalkyl, cycloalkenyl, substituted cycloalkenyl, acyl, acylamino, acyloxyl, aminoacyl, aminoacyloxyl, oxyaminoacyl, azido, cyano, halogen, hydroxyl, keto, thioketo, carboxyl, carboxylalkyl, thioaryloxyl, thioheteroaryloxyl, thioheterocyclooxyl, thiol, thioalkoxyl, substituted thioalkoxyl, aryl, aryloxyl, heteroaryl, heteroaryloxyl, heterocyclic, heterocyclooxyl, hydroxyamino, alkoxyamino, nitro, —SO-alkyl, —SO-aryl, —SO-heteroaryl, —SO2-alkyl, —SO2-aryl, —SO2-heteroaryl, and —NRaRb, wherein Ra and Rb may be the same or different and are chosen from hydrogen, optionally substituted alkyl, cycloalkyl, alkenyl, cycloalkenyl, alkynyl, aryl, heteroaryl and heterocyclic group.

“Alkylamino” as used herein denotes amino groups which have been substituted with one or two alkyl radicals. Preferred are “lower N-alkylamino” radicals having alkyl portions having 1 to 6 carbon atoms. Preferred lower alkylamino may be mono or dialkylamino such as N-methylamino, N-ethylamino, N,N-dimethylamino, N,N-diethylamino or the like.

“Alkylaminoalkyl” as used herein embraces radicals having one or more alkyl radicals attached to an aminoalkyl radical.

“Alkylaminocarbonyl” as used herein denotes an aminocarbonyl group which has been substituted with one or two alkyl radicals on the amino nitrogen atom. Preferred are “N-alkylaminocarbonyl” “N,N-dialkylaminocarbonyl” radicals. More preferred are “lower N-alkylaminocarbonyl” “lower N,N-dialkylaminocarbonyl” radicals with lower alkyl portions as defined above.

“Alkylcarbonyl”, “arylcarbonyl” and “aralkylcarbonyl” as used herein include radicals having alkyl, aryl and aralkyl radicals, as defined above, attached via an oxygen atom to a carbonyl radical. Examples of such radicals include, without limitation, substituted or unsubstituted methylcarbonyl, ethylcarbonyl, phenylcarbonyl and benzylcarbonyl.

“Alkylsulfinyl” as used herein embraces radicals containing a linear or branched alkyl radical, of one to ten carbon atoms, attached to a divalent —S(═O)— radical. More preferred alkylsulfinyl radicals are “lower alkylsulfinyl” radicals having alkyl radicals of one to six carbon atoms. Examples of such lower alkylsulfinyl radicals include, without limitation, methylsulfinyl, ethylsulfinyl, butylsulfinyl and hexylsulfinyl.

“Alkylsulfonyl” as used herein embraces alkyl radicals attached to a sulfonyl radical, where alkyl is defined as above. More preferred alkylsulfonyl radicals are “lower alkylsulfonyl” radicals having one to six carbon atoms. Examples of such lower alkylsulfonyl radicals include, without limitation, methylsulfonyl, ethylsulfonyl and propylsulfonyl. The “alkylsulfonyl” radicals may be further substituted with one or more halo atoms, such as fluoro, chloro or bromo, to provide haloalkylsulfonyl radicals.

“Alkylthio” as used herein embraces radicals containing a linear or branched alkyl radical, of one to about ten carbon atoms attached to a divalent sulfur atom. More preferred alkylthio radicals are “lower alkylthio” radicals having alkyl radicals of one to six carbon atoms. Examples of such lower alkylthio radicals are methylthio, ethylthio, propylthio, butylthio and hexylthio.

“Alkylthioalkyl” as used herein embraces radicals containing an alkylthio radical attached through the divalent sulfur atom to an alkyl radical of one to about ten carbon atoms. More preferred alkylthioalkyl radicals are “lower alkylthioalkyl” radicals having alkyl radicals of one to six carbon atoms. Examples of such lower alkylthioalkyl radicals include, without limitation, methylthiomethyl.

“Alkylene” as used herein refers to a diradical of a branched or unbranched saturated hydrocarbon chain, preferably having from 1 to 40 carbon atoms, more preferably 1 to 10 carbon atoms, even more preferably 1 to 6 carbon atoms. This term is exemplified by groups such as methylene (—CH2-), ethylene (—CH2CH2-), the propylene isomers (e.g. —CH2CH2CH2- and —CH(CH3)CH2-), and the like.

“Substituted alkylene” as used herein refers to: (1) an alkylene group as defined above having from 1 to 5 substituents selected from a member of the group consisting of alkoxyl, substituted alkoxyl, cycloalkyl, substituted cycloalkyl, cycloalkenyl, substituted cycloalkenyl, acyl, acylamino, acyloxyl, aminoacyl, aminoacyloxyl, oxyacylamino, azido, cyano, halogen, hydroxyl, keto, thioketo, carboxyl, carboxylalkyl, thiol, thioalkoxyl, substituted thioalkoxyl, aryl, aryloxyl, thioaryloxyl, heteroaryl, heteroaryloxyl, thioheteroaryloxyl, heterocyclic, heterocyclooxyl, thioheterocyclooxyl, nitro, and —NRaRb, wherein Ra and Rb may be the same or different and are chosen from hydrogen, optionally substituted alkyl, cycloalkyl, alkenyl, cycloalkenyl, alkynyl, aryl, heteroaryl and heterocyclic. Additionally, such substituted alkylene groups include, without limitation, those where 2 substituents on the alkylene group are fused to form one or more cycloalkyl, substituted cycloalkyl, cycloalkenyl, substituted cycloalkenyl, aryl, heterocyclic or heteroaryl groups fused to the alkylene group; (2) an alkylene group as defined above that is interrupted by 1-20 atoms independently chosen from oxygen, sulfur and NRa, where Ra is chosen from hydrogen, optionally substituted alkyl, cycloalkyl, alkenyl, cycloalkenyl, alkenyl, cycloalkenyl, alkynyl, aryl, heteroaryl and heterocyclic, or groups selected from carbonyl, carboxyester, carboxyamide and sulfonyl; or (3) an alkylene group as defined above that has both from 1 to 5 substituents as defined above and is also interrupted by 1 to 20 atoms as defined above. Examples of substituted alkylenes are chloromethylene (—CH(C1)-), aminoethylene (—CH(NH2)CH2-), 2-carboxypropylene isomers (—CH2CH(CO2H)CH2-), ethoxyethyl (—CH2CH2O—CH2CH2-), ethylmethylaminoethyl (—CH2CH2N(CH3)CH2CH2-), 1-ethoxy-2-(2-ethoxy-ethoxy)ethane (—CH2CH2O—CH2CH2-OCH2CH2-OCH2CH2-), and the like.

“Alkynyl” as used herein is intended to include hydrocarbon chains of either a straight or branched configuration and one or more triple carbon-carbon bonds which may occur in any stable point along the chain, such as ethynyl, propynyl and the like. For example, alkynyl refers to an unsaturated acyclic hydrocarbon radical in so much as it contains one or more triple bonds, such radicals containing about 2 to about 40 carbon atoms, preferably having from about 2 to about 10 carbon atoms and more preferably having 2 to about 6 carbon atoms. Non-limiting examples of preferred alkynyl radicals include, ethynyl, propynyl, butyn-1-yl, butyn-2-yl, pentyn-1-yl, pentyn-2-yl, 3-methylbutyn-1-yl, hexyn-1-yl, hexyn-2-yl, hexyn-3-yl, 3,3-dimethylbutyn-1-yl radicals and the like.

“Alicyclic hydrocarbon” as used herein means a aliphatic radical in a ring with 3 to about 10 carbon atoms, and preferably from 3 to about 6 carbon atoms. Examples of preferred alicyclic radicals include, without limitation, cyclopropyl, cyclopropylenyl, cyclobutyl, cyclopentyl, cyclohexyl, 2-cyclohexen-1-ylenyl, cyclohexenyl and the like.

“Alkoxyalkyl” as used herein embraces alkyl radicals having one or more alkoxy radicals attached to the alkyl radical, that is, to form monoalkoxyalkyl and dialkoxyalkyl radicals. The “alkoxy” radicals may be further substituted with one or more halo atoms, such as fluoro, chloro or bromo, to provide haloalkoxy radicals. More preferred haloalkoxy radicals are “lower haloalkoxy” radicals having one to six carbon atoms and one or more halo radicals. Examples of such radicals include, without limitation, fluoromethoxy, chloromethoxy, trifluoromethoxy, trifluoromethoxy, fluoroethoxy and fluoropropoxy. Further, “alkoxycarbonyl” means a radical containing an alkoxy radical, as defined above, attached via an oxygen atom to a carbonyl radical. More preferred are “lower alkoxycarbonyl” radicals with alkyl portions having 1 to 6 carbons. Examples of such lower alkoxycarbonyl (ester) radicals include, without limitation, substituted or unsubstituted methoxycarbonyl, ethoxycarbonyl, propoxycarbonyl, butoxycarbonyl and hexyloxycarbonyl.

“Aminoalkyl” as used herein embraces alkyl radicals substituted with amino radicals. More preferred are “lower aminoalkyl” radicals. Examples of such radicals include, without limitation, aminomethyl, aminoethyl, and the like.

“Aminocarbonyl” as used herein denotes an amide group of the formula —C(═O)NH2.

“Aralkoxy” as used herein embraces aralkyl radicals attached through an oxygen atom to other radicals.

“Aralkoxyalkyl” as used herein embraces aralkoxy radicals attached through an oxygen atom to an alkyl radical.

“Aralkyl” as used herein embraces aryl-substituted alkyl radicals such as benzyl, diphenylmethyl, triphenylmethyl, phenylethyl, and diphenylethyl. The aryl in said aralkyl may be additionally substituted with halo, alkyl, alkoxy, halkoalkyl and haloalkoxy.

“Aralkylamino” as used herein embraces aralkyl radicals attached through an nitrogen atom to other radicals.

“Aralkylthio” as used herein embraces aralkyl radicals attached to a sulfur atom.

“Aralkylthioalkyl” as used herein embraces aralkylthio radicals attached through a sulfur atom to an alkyl radical.

“Aromatic hydrocarbon radical” as used herein means 4 to about 16 carbon atoms, preferably 6 to about 12 carbon atoms, more preferably 6 to about 10 carbon atoms. Examples of preferred aromatic hydrocarbon radicals include, without limitation, phenyl, naphthyl, and the like.

“Aroyl” as used herein embraces aryl radicals with a carbonyl radical as defined above. Examples of aroyl include, without limitation, benzoyl, naphthoyl, and the like and the aryl in said aroyl may be additionally substituted.

“Arylamino” as used herein denotes amino groups which have been substituted with one or two aryl radicals, such as N-phenylamino. Arylamino radicals may be further substituted on the aryl ring portion of the radical.

“Aryloxyalkyl” as used herein embraces radicals having an aryl radical attached to an alkyl radical through a divalent oxygen atom.

“Arylthioalkyl” as used herein embraces radicals having an aryl radical attached to an alkyl radical through a divalent sulfur atom.

“Carbonyl” whether used alone or with other terms, such as “alkoxycarbonyl”, denotes —(C═O)—.

“Carboxy” or “carboxyl”, whether used alone or with other terms, such as “carboxyalkyl”, denotes —CO2H.

“Carboxyalkyl” as used herein embraces alkyl radicals substituted with a carboxy radical. More preferred are “lower carboxyalkyl” which embrace lower alkyl radicals as defined above, and may be additionally substituted on the alkyl radical with halo. Examples of such lower carboxyalkyl radicals include, without limitation, carboxymethyl, carboxyethyl and carboxypropyl.

“Cycloalkenyl” as used herein embraces partially unsaturated carbocyclic radicals having three to twelve carbon atoms. More preferred cycloalkenyl radicals are “lower cycloalkenyl” radicals having four to about eight carbon atoms. Examples of such radicals include, without limitation, cyclobutenyl, cyclopentenyl and cyclohexenyl.

“Cycloalkyl” as used herein embraces saturated carbocyclic radicals having three to twelve carbon atoms. More preferred cycloalkyl radicals are “lower cycloalkyl” radicals having three to about eight carbon atoms. Examples of such radicals include, without limitation, cyclopropyl, cyclobutyl, cyclopentyl and cyclohexyl.

“Hydroxyalkyl” as used herein embraces linear or branched alkyl radicals having one to about twenty carbon atoms any one of which may be substituted with one or more hydroxyl radicals. Preferred hydroxyalkyl radicals are “lower hydroxyalkyl” radicals having one to six carbon atoms and one or more hydroxyl radicals. Non-limiting examples of such radicals include hydroxymethyl, hydroxyethyl, hydroxypropyl, hydroxybutyl and hydroxyhexyl.

“Sulfamyl”, “aminosulfonyl” and “sulfonamidyl” as used herein denote NH2O2S—.

“Sulfonyl”, whether used alone or linked to other terms such as alkylsulfonyl, denotes respectively divalent radicals —SO2-.

“Alkenyl” as used herein is intended to include hydrocarbon chains of either a straight or branched configuration and one or more unsaturated carbon-carbon bonds which may occur in any stable point along the chain. For example, alkenyl refers to an unsaturated acyclic hydrocarbon radical in so much as it contains at least one double bond. Such radicals containing from about 2 to about 40 carbon atoms, preferably from about 2 to about 10 carbon atoms and more preferably about 2 to about 6 carbon atoms. Non-limiting examples of preferred alkenyl radicals include propylenyl, buten-1-yl, isobutenyl, penten-1-yl, 2-2-methylbuten-1-yl, 3-methylbuten-1-yl, hexen-1-yl, hepten-1-yl, and octen-1-yl, and the like

“Alkoxyl” as used herein represents an alkyl group of indicated number of carbon atoms attached through an oxygen bridge. “Alkoxy” and “alkyloxy” embrace linear or branched oxy-containing radicals each having alkyl portions of one to about ten carbon atoms. More preferred alkoxy radicals are “lower alkoxy” radicals having one to six carbon atoms. Examples of such radicals include, without limitation, methoxy, ethoxy, propoxy, butoxy and tert-butoxy.

“Aryl” as used herein refers to an unsaturated aromatic carbocyclic group of from 6 to 20 carbon atoms having a single ring (e.g., phenyl) or multiple condensed (fused) rings (e.g., naphthyl or anthryl). “aryl” embraces aromatic radicals such as phenyl, naphthyl, tetrahydronaphthyl, indane and biphenyl. Unless otherwise constrained by the definition for the aryl substituent, such aryl groups can optionally be substituted with from 1 to 5 substituents selected from a member of the group consisting of acyloxyl, hydroxyl, thiol, acyl, alkyl, alkoxyl, alkenyl, alkynyl, cycloalkyl, cycloalkenyl, substituted alkyl, substituted alkoxyl, substituted alkenyl, substituted alkynyl, substituted cycloalkyl, substituted cycloalkenyl, aminoacyl, acylamino, alkaryl, aryl, aryloxyl, azido, carboxyl, carboxylalkyl, cyano, halo, nitro, heteroaryl, heteroaryloxyl, heterocyclic, heterocyclooxyl, aminoacyloxyl, oxyacylamino, thioalkoxyl, substituted thioalkoxyl, thioaryloxyl, thioheteroaryloxyl, —SO-alkyl, —SO-substituted alkyl, —SO-aryl, —SO-heteroaryl, —SO2-alkyl, —SO2-substituted alkyl, —SO2-aryl, —SO2-heteroaryl, trihalomethyl, NRaRb, wherein Ra and Rb may be the same or different and are chosen from hydrogen, optionally substituted alkyl, cycloalkyl, alkenyl, cycloalkenyl, alkynyl, aryl, heteroaryl and heterocyclic. Preferred aryl substituents include, without limitation, without limitation, alkyl, alkoxyl, halo, cyano, nitro, trihalomethyl, and thioalkoxy (i.e., —S-alkyl).

“N-arylaminoalkyl” and “N-aryl-N-alkyl-aminoalkyl” as used herein denote amino groups which have been substituted with one aryl radical or one aryl and one alkyl radical, respectively, and having the amino group attached to an alkyl radical. Examples of such radicals include, without limitation, N-phenylaminomethyl and N-phenyl-N-methylaminomethyl.

“Carbocycle” or “carbocyclic group” as used herein is intended to mean any stable 3 to 7 membered monocyclic or bicyclic or 7 to 14 membered bicyclic or tricyclic or an up to 26 membered polycyclic carbon ring, any of which may be saturated, partially unsaturated, or aromatic.

“Substituted carbocycle” or “substituted carbocyclic group” as used herein refers to carbocyclic groups having from 1 to 5 substituents selected from a member of the group consisting of alkoxyl, substituted alkoxyl, cycloalkyl, cycloalkenyl, substituted cycloalkenyl, acyl, acylamino, acyloxyl, amino, aminoacyl, aminoacyloxyl, oxyaminoacyl, azido, cyano, halogen, hydroxyl, keto, thioketo, carboxyl, carboxylalkyl, thioaryloxyl, thioheteroaryloxyl, thioheterocyclooxyl, thiol, thioalkoxyl, substituted thioalkoxyl, aryl, aryloxyl, heteroaryl, heteroaryloxyl, heterocyclic, heterocyclooxyl, hydroxyamino, alkoxyamino, nitro, —SO-alkyl, —SO-substituted alkyl, —SO-aryl, —SO-heteroaryl, —SO2-alkyl, —SO2-substituted alkyl, —SO2-aryl, —SO2-heteroaryl, and NRaRb, wherein Ra and Rb may be the same or different and are chosen from hydrogen, optionally substituted alkyl, cycloalkyl, alkenyl, cycloalkenyl, alkynyl, aryl, heteroaryl and heterocyclic. Preferred examples of carbocyclic groups include, without limitation, members selected from the group consisting of adamantyl, anthracenyl, benzamidyl, benzyl, bicyclo[2.2.1]heptanyl, bicyclo[2.2.1]hexanyl, bicyclo[2.2.2]octanyl, bicyclo[3.2.0]heptanyl, bicyclo[4.3.0]nonanyl, bicyclo[4.4.0]decanyl, biphenyl, biscyclooctyl, cyclobutanyl (cyclobutyl), cyclobutenyl, cycloheptanyl (cycloheptyl), cycloheptenyl, cyclohexanedionyl, cyclohexenyl, cyclohexyl, cyclooctanyl, cyclopentadienyl, cyclopentanedionyl, cyclopentenyl, cyclopentyl, cyclopropyl, decalinyl, 1,2-diphenylethanyl, indanyl, 1-indanonyl, indenyl, naphthyl, napthlalenyl, phenyl, resorcinolyl, stilbenyl, tetrahydronaphthyl (tetralin), tetralinyl, tetralonyl, tricyclododecanyl, and the like.

“Cycloalkyl” as used herein is intended to include saturated ring groups, including mono-, bi- or poly-cyclic ring systems, such as, without limitation, cyclopropyl, cyclobutyl, cyclopentyl, cyclohexyl, cycloheptyl, cyclooctyl, and adamantyl. “Bicycloalkyl” is intended to include saturated bicyclic ring groups such as, without limitation, [3.3.0]bicyclooctane, [4.3.0]bicyclononane, [4.4.0]bicyclodecane (decalin), [2.2.2]bicyclooctane, and so forth.

“Halo” or “halogen” as used herein refers to fluoro, chloro, bromo and iodo; and “counterion” is used to represent a small, negatively charged species such as chloride, bromide, hydroxide, acetate, sulfate and the like.

“Haloalkyl” as used herein is intended to include both branched and straight-chain saturated aliphatic hydrocarbon groups having the specified number of carbon atoms, substituted with 1 or more halogen. Haloalkyl embraces radicals wherein any one or more of the alkyl carbon atoms is substituted with halo as defined above. Specifically embraced are monohaloalkyl, dihaloalkyl and polyhaloalkyl radicals. A monohaloalkyl radical, for one example, may have either an iodo, bromo, chloro or fluoro atom within the radical. Dihalo and polyhaloalkyl radicals may have two or more of the same halo atoms or a combination of different halo radicals. “Lower haloalkyl” embraces radicals having 1-6 carbon atoms. Non-limiting examples of haloalkyl radicals include fluoromethyl, difluoromethyl, trifluoromethyl, chloromethyl, dichloromethyl, trichloromethyl, pentafluoroethyl, heptafluoropropyl, difluorochloromethyl, dichlorofluoromethyl, difluoroethyl, difluoropropyl, dichloroethyl and dichloropropyl.

“Heterocycle” or “heterocyclic group” as used herein refers to a saturated or unsaturated group having a single ring, multiple condensed rings or multiple joined/bonded rings, from 1 to 40 carbon atoms and from 1 to 10 hetero ring atoms, preferably 1 to 4 hetero ring atoms, selected from nitrogen, sulfur, phosphorus, and/or oxygen. Preferably, “heterocycle” or “heterocyclic group” means a stable 5 to 7 membered monocyclic or bicyclic or 7 to 10 membered bicyclic heterocyclic ring that may be saturated, partially unsaturated, or aromatic, and that comprises carbon atoms and from 1 to 4 heteroatoms independently selected from a member of the group consisting of nitrogen, oxygen and sulfur and wherein the nitrogen and sulfur heteroatoms are optionally be oxidized and the nitrogen heteroatom may optionally be quaternized, and including any bicyclic group in which any of the above-defined heterocyclic rings is fused to a benzene ring. The heterocyclic groups may be substituted on carbon or on a nitrogen, sulfur, phosphorus, and/or oxygen heteroatom so long as the resulting compound is stable. Unless otherwise constrained by the definition for the heterocyclic substituent, such heterocyclic groups can be optionally substituted with 1 to 5, and preferably 1 to 3 substituents. Suitable, but non-limiting, examples of such substituents include members selected from the group consisting of alkoxyl, substituted alkoxyl, cycloalkyl, substituted cycloalkyl, cycloalkenyl, substituted cycloalkenyl, acyl, acylamino, acyloxyl, aminoacyl, aminoacyloxyl, oxyaminoacyl, cyano, halogen, hydroxyl, keto, thioketo, carboxyl, carboxylalkyl, thioaryloxyl, thioheteroaryloxyl, thioheterocyclooxyl, thiol, thioalkoxyl, substituted thioalkoxyl, aryl, aryloxyl, heteroaryl, heteroaryloxyl, heterocyclic, heterocyclooxyl, hydroxyamino, alkoxyamino, nitro, —SO-alkyl, —SO-substituted alkyl, —SO-aryl, —SO-heteroaryl, —SO2-alkyl, —SO2-substituted alkyl, —SO2-aryl, —SO, -heteroaryl, and NRaRb, wherein Ra and Rb may be the same or different and are chosen from hydrogen, optionally substituted alkyl, cycloalkyl, alkenyl, cycloalkenyl, alkynyl, aryl, heteroaryl and heterocyclic.

Preferred examples of such heterocyclic groups include, without limitation, acridinyl, acridonyl, adeninyl, alkylpyridinyl, alloxanyl, alloxazinyl, anthracenyl, anthranilyl, anthraquinonyl, anthrenyl, ascorbyl, azaazulenyl, azabenzanthracenyl, azabenzanthrenyl, azabenzonaphthenyl, azabenzophenanthrenyl, azachrysenyl, azacyclazinyl, azaindolyl, azanaphthacenyl, azanaphthalenyl, azaphenoxazinyl, azapinyl, azapurinyl, azapyrenyl, azatriphenylenyl, azepinyl, azetidinedionyl, azetidinonyl, azetidinyl, azinoindolyl, azinopyrrolyl, azinyl, aziridinonyl, aziridinyl, azirinyl, azocinyl, azoloazinyl, azolyl, barbituric acid, benzacridinyl, benzazapinyl, benzazinyl, benzimidazolethionyl, benzimidazolonyl, benzimidazolyl, benzisothiazolyl, benzisoxazolyl, benzocinnolinyl, benzodiazocinyl, benzodioxanyl, benzodioxolanyl, benzodioxolyl, benzofuranyl (benzofuryl), benzofuroxanyl, benzonaphthyridinyl, benzopyranonyl (benzopyranyl), benzopyridazinyl, benzopyronyl, benzoquinolinyl, benzoquinolizinyl, benzothiadiazinyl, benzothiazepinyl, benzothiazinyl, benzothiazolyl, benzothiepinyl, benzothiophenyl, benzotriazepinonyl, benzotriazolyl, benzoxadizinyl, benzoxazinyl, benzoxazolinonyl, benzoxazolyl, benzylisoquinolinyl, beta-carbolinyl, biotinyl, bipyridinyl, butenolidyl, butyrolactonyl, caprolactamyl, carbazolyl, 4a H-carbazolyl, carbolinyl, catechinyl, chromanyl, chromenopyronyl, chromonopyranyl, chromylenyl, cinnolinyl, coumarinyl, coumaronyl, decahydroquinolinyl, decahydroquinolonyl, depsidinyl, diazaanthracenyl, diazaphenanthrenyl, diazepinyl, diazinyl, diaziridinonyl, diaziridinyl, diazirinyl, diazocinyl, dibenzazepinyl, dibenzofuranyl, dibenzothiophenyl, dibenzoxazepinyl, dichromylenyl, dihydrobenzimidazolyl, dihydrobenzothiazinyl, dihydrofuranyl, dihydroisocoumarinyl, dihydroisoquinolinyl, dihydrooxazolyl, dihydropyranyl, dihydropyridazinyl, dihydropyridinyl, dihydropyridonyl, dihydropyrimidinyl, dihydropyronyl, dihydrothiazinyl, dihydrothiopyranyl, dihydroxybenzenyl, dimethoxybenzenyl, dimethylxanthinyl, dioxadiazinyl, dioxanthylenyl, dioxanyl, dioxenyl, dioxepinyl, dioxetanyl, dioxinonyl, dioxinonyl, dioxiranyl, dioxolanyl, dioxolonyl, dioxolyl, dioxopiperazinyl, diprylenyl, dipyrimidopyrazinyl, dithiadazolyl, dithiazolyl, 2H,6H-1,5,2-dithiazinyl, dithietanyl, dithiolanyl, dithiolenyl, dithiolyl, enantholactamyl, episulfonyl, flavanyl, flavanyl, flavinyl, flavonyl, fluoranyl, fluorescienyl, furandionyl, furanochromanyl, furanonyl, furanoquinolinyl, furanyl (furyl), furazanyl, furfuryl, furopyranyl, furopyrimidinyl, furopyronyl, furoxanyl, glutarimidyl, glycocyamidinyl, guaninyl, heteroazulenyl, hexahydropyrazinoisoquinolinyl, hexahydropyridazinyl, homophthalimidyl, hydantoinyl, hydrofuranyl, hydrofurnanonyl, hydroimidazolyl, hydroindolyl, hydropyranyl, hydropyrazinyl, hydropyrazolyl, hydropyridazinyl, hydropyridinyl, hydropyrimidinyl, hydropyrrolyl, hydroquinolinyl, hydrothiochromenyl, hydrothiophenyl, hydrotriazolyl, hydroxytrizinyl, imidazolethionyl, imidazolidinyl, imidazolinyl, imidazolonyl, imidazolyl, imidazoquinazolinyl, imidazothiazolyl, indazolebenzopyrazolyl, indazolyl, 1H-indazolyl, indolenyl, indolinyl, indolizidinyl, indolizinyl, indolonyl, indolyl, 3H-indolyl, indoxazenyl, inosinyl, isatinyl, isatogenyl, isoalloxazinyl, isobenzofurandionyl, isobenzofuranyl, isochromanyl, isoflavonyl, isoindolinyl (isoindolyl), isoindolobenzazepinyl, isoquinolinyl, isoquinuclidinyl, isothiazolyl, isoxazolidinyl, isoxazolinonyl, isoxazolinyl, isoxazolonyl, isoxazolyl, lactamyl, lactonyl, lumazinyl, maleimidyl, methylbenzamidyl, methylbenzoyleneureayl, methyldihydrouracilyl, methyldioxotetrahydropteridinyl, methylpurinyl, methylthyminyl, methylthyminyl, methyluracilyl, methylxanthinyl, monoazabenzonaphthenyl, morpholinyl (morpholino), naphthacenyl, naphthalenyl, naphthimidazolyl, naphthimidazopyridinedionyl, naphthindolizinedionyl, naphthodihydropyranyl, naphthofuranyl, naphthothiophenyl, naphthylpyridinyl, naphthyridinyl, octahydroisoquinolinyl, octylcarboxamidobenzenyl, oroticyl, oxadiazinyl, oxadiazolyl, oxathianyl, oxathiazinonyl, oxathietanyl, oxathiiranyl, oxathiolanyl, oxatriazolyl, oxazinonyl, oxaziranyl, oxaziridinyl, oxazolidinonyl, oxazolidinyl, oxazolidonyl, oxazolinonyl, oxazolinyl, oxazolonyl, oxazolopyrimidinyl, oxazolyl, oxepinyl, oxetananonyl, oxetanonyl, oxetanyl, oxindolyl, oxiranyl, oxolenyl, pentazinyl, pentazolyl, perhydroazolopyridinyl, perhydrocinnolinyl, perhydroindolyl, perhydropyrroloazinyl, perhydropyrrolooxazinyl, perhydropyrrolothiazinyl, perhydrothiazinonyl, perimidinyl, petrazinyl, phenanthraquinonyl, phenanthridinyl, phenanthrolinyl, phenarsazinyl, phenazinyl, phenothiazinyl, phenoxanthinyl, phenoxazinyl, phenoxazonyl, phthalazinyl, phthalideisoquinolinyl, phthalimidyl, phthalonyl, piperazindionyl, piperazinodionyl, piperazinyl, piperidinyl, piperidonyl, 4-piperidonyl, polyoxadiazolyl, polyquinoxalinyl, prolinyl, prylenyl, pteridinyl, pterinyl, purinyl, pyradinyl, pyranoazinyl, pyranoazolyl, pyranonyl, pyranopyradinyl, pyranopyrandionyl, pyranopyridinyl, pyranoquinolinyl, pyranyl, pyrazinyl, pyrazolidinyl, pyrazolidonyl, pyrazolinonyl, pyrazolinyl, pyrazolobenzodiazepinyl, pyrazolonyl, pyrazolopyridinyl, pyrazolopyrimidinyl, pyrazolotriazinyl, pyrazolyl, pyrenyl, pyridazinyl, pyridazonyl, pyridinethionyl, pyridinonaphthalenyl, pyridinopyridinyl, pyridocolinyl, pyridoindolyl, pyridopyrazinyl, pyridopyridinyl, pyridopyrimidinyl, pyridopyrrolyl, pyridoquinolinyl, pyridyl (pyridinyl), pyrimidinethionyl, pyrimidinyl, pyrimidionyl, pyrimidoazepinyl, pyrimidopteridinyl, pyronyl, pyrrocolinyl, pyrrolidinyl, 2-pyrrolidinyl, pyrrolinyl, pyrrolizidinyl, pyrrolizinyl, pyrrolobenzodiazepinyl, pyrrolodiazinyl, pyrrolonyl, pyrrolopyrimidinyl, pyrroloquinolonyl, pyrrolyl, 2H-pyrrolyl, quinacridonyl, quinazolidinyl, quinazolinonyl, quinazolinyl, quinolinyl, quinolizidinyl, quinolizinyl, 4H-quinolizinyl, quinolonyl, quinonyl, quinoxalinyl, quinuclidinyl, quinuclidinyl, rhodaminyl, spirocoumaranyl, succinimidyl, sulfolanyl, sulfolenyl, sultamyl, sultinyl, sultonyl, sydononyl, tetrahydrofuranyl, tetrahydroisoquinolinyl, tetrahydrooxazolyl, tetrahydropyranyl, tetrahydropyrazinyl, tetrahydropyridazinyl, tetrahydropyridinyl, tetrahydroquinolinyl, tetrahydroquinoxalinyl, tetrahydrothiapyranyl, tetrahydrothiazolyl, tetrahydrothiophenyl, tetrahydrothiopyranonyl, tetrahydrothiopyranyl, tetraoxanyl, tetrazepinyl, tetrazinyl, tetrazolyl, tetronyl, thiabenzenyl, thiachromanyl, thiadecalinyl, thiadiazinyl, 6H-1,2,5-thiadiazinyl, thiadiazolinyl, thiadiazolyl, thiadioxazinyl, thianaphthenyl, thianthrenyl, thiapyranyl, thiapyronyl, thiatriazinyl, thiatriazolyl, thiazepinyl, thiazetidinyl, thiazinyl, thiaziridinyl, thiazolidinonyl, thiazolidinyl, thiazolinonyl, thiazolinyl, thiazolobenzimidazolyl, thiazolopyridinyl, thiazolyl, thienopryidinyl, thienopyrimidinyl, thienopyrrolyl, thienothiophenyl, thienyl, thiepinyl, thietanyl, thiiranyl, thiochromenyl, thiocoumarinyl, thiolanyl, thiolenyl, thiolyl, thiophenyl, thiopyranyl, thyminyl, triazaanthracenyl, triazepinonyl, triazepinyl, triazinoindolyl, triazinyl, triazolinedionyl, triazolinyl, triazolopyridinyl, triazolopyrimidinyl, triazolyl, trioxanyl, triphenodioxazinyl, triphenodithiazinyl, trithiadiazepinyl, trithianyl, trixolanyl, trizinyl, tropanyl, uracilyl, xanthenyl, xanthinyl, xanthonyl, xanthydrolyl, xylitolyl, and the like as well as N-alkoxy-nitrogen containing heterocycles. Preferred heterocyclic groups include, without limitation, members of the group consisting of acridinyl, aziridinyl, azocinyl, azepinyl, benzimidazolyl, benzodioxolanyl, benzofuranyl, benzothiophenyl, carbazole, 4a H-carbazole, chromanyl, chromenyl, cinnolinyl, decahydroquinolinyl, dioxoindolyl, furazanyl, furyl, furfuryl, imidazolidinyl, imidazolinyl, imidazolyl, 1H-indazolyl, indolenyl, indolinyl, indolizinyl, indolyl, 3H-indolyl, isobenzofuranyl, isochromanyl, isoindolinyl, isoindolyl, isoquinolinyl, isothiazolyl, isoxazolyl, morpholinyl, naphthalenyl, naphthyridinyl, norbornanyl, norpinanyl, octahydroisoquinolinyl, oxazolidinyl, oxazolyl, oxiranyl, perimidinyl, phenanthridinyl, phenanthrolinyl, phenarsazinyl, phenazinyl, phenothiazinyl, phenoxathiinyl, phenoxazinyl, phenyl, phthalazinyl, piperazinyl, piperidinyl, 4-piperidonyl, piperidyl, pteridinyl, purinyl, pyranyl, pyrazinyl, pyrazolidinyl, pyrazolinyl, pyrazolyl, pyrenyl, pyridazinyl, pyridinyl, pyridyl, pyridyl, pyrimidinyl, pyrrolidinyl, 2-pyrrolidonyl, pyrrolonyl, pyrrolyl, 2H-pyrrolyl, quinazolinyl, 4H-quinolizinyl, quinolinyl, quinoxalinyl, quinuclidinyl, β-carbolinyl, tetrahydrofuranyl, tetrahydroisoquinolinyl, tetrahydroquinolinyl, tetrazolyl, 6H-1,2,5-thiadiazinyl, 2H-, 6H-1,5,2-dithiazinyl, thianthrenyl, thiazolyl, thienyl, thiophenyl, triazinyl, xanthenyl, xanthinyl, and the like.

“Pharmaceutically acceptable derivative” or “prodrug” as used herein means any pharmaceutically acceptable salt, ester, salt of an ester, or other derivative of a compound of the present invention which, upon administration to a recipient, is capable of providing (directly or indirectly) a compound of this invention. The term “prodrug”, as employed herein, denotes a compound that is a drug precursor which, upon administration to a subject, undergoes chemical conversion by metabolic or chemical processes to yield a pharmaceutically active compound. Particularly favored derivatives and prodrugs are those that increase the bioavailability of the compounds of this invention when such compounds are administered to a mammal (e.g., by allowing an orally administered compound to be more readily absorbed into the blood) or that enhance delivery of the parent compound to a biological compartment (e.g., the brain or lymphatic system) relative to the parent species. Prodrugs are considered to be any covalently bonded carriers which release the active parent drug according to Formula I or II in vivo when such prodrug is administered to a mammalian subject. Preferred prodrugs include, without limitation, derivatives where a group that enhances aqueous solubility or active transport through the gut membrane is appended to the structure of Formula I or II. Prodrugs of the compounds of Formula I or II are prepared by modifying functional groups present in the compounds in such a way that the modifications are cleaved, either in routine manipulation or in vivo, to the parent compounds. Prodrugs include compounds of Formula I or II wherein hydroxyl, amino, sulfhydryl, or carboxyl groups are bonded to any group that, when administered to a mammalian subject, cleaves to form a free hydroxyl, amino, sulfhydryl, or carboxyl group, respectively. Examples of prodrugs include, but are not limited to, acetate, formate and benzoate derivatives of alcohol and amine functional groups in the compounds of Formula I or II, and the like. A discussion of prodrugs is provided in T. Higuchi and V. Stella, Pro-drugs as Novel Delivery Systems (1987) 14 of the A.C.S. Symposium Series, and in Bioreversible Carriers in Drug Design, (1987) Edward B. Roche, ed., American Pharmaceutical Association and Pergamon Press, both of which are incorporated herein by reference.

“Solvate” means a physical association of a compound described herein with one or more solvent molecules. This physical association involves varying degrees of ionic and covalent bonding, including hydrogen bonding. In certain instances the solvate will be capable of isolation, for example when one or more solvent molecules are incorporated in the crystal lattice of the crystalline solid. “Solvate” encompasses both solution-phase and isolatable solvates. Non-limiting examples of preferred solvates include ethanolates, methanolates, and the like. “Hydrate” is a solvate wherein the solvent molecule is H₂O.

“Pharmaceutically acceptable salts” as used herein refer to derivatives of the disclosed compounds wherein the parent compound of Formula I or II is modified by making acid or base salts of the compound of Formula I or II. Examples of pharmaceutically acceptable salts include, but are not limited to, mineral or organic acid salts of basic residues such as amines; alkali or organic salts of acidic residues such as carboxylic acids; and the like. The pharmaceutically acceptable salts of the compounds of Formula I or II include the conventional nontoxic salts or the quaternary ammonium salts of the compounds of Formula I or II formed, for example, from nontoxic inorganic or organic acids. For example, such conventional non-toxic salts include, without limitation, those derived from inorganic acids such as acetic, 2-acetoxybenzoic, adipic, alginic, ascorbic, aspartic, benzoic, benzenesulfonic, bisulfic, butyric, citric, camphoric, camphorsulfonic, cyclopentanepropionic, digluconic, dodecylsulfanilic, ethane disulfonic, ethanesulfonilic, fumaric, glucoheptanoic, glutamic, glycerophosphic, glycolic, hemisulfanoic, heptanoic, hexanoic, hydrochloric, hydrobromic, hydroiodic, 2-hydroxyethanesulfonoic, hydroxymaleic, isethionic, lactic, malic, maleic, methanesulfonic, 2-naphthalenesulfonilic, nicotinic, nitric, oxalic, palmic, pamoic, pectinic, persulfanilic, phenylacetic, phosphoric, propionic, pivalic, propionate, salicylic, succinic, stearic, sulfuric, sulfamic, sulfanilic, tartaric, thiocyanic, toluenesulfonic, tosylic, undecanoatehydrochloric, and the like. The pharmaceutically acceptable salts of the present invention can be synthesized from the compounds of Formula I or II which contain a basic or acidic moiety by conventional chemical methods, for example, by reacting the free base or acid with stoichiometric amounts of the appropriate base or acid, respectively, in water or in an organic solvent, or in a mixture of the two (nonaqueous media like ether, ethyl acetate, ethanol, isopropanol, or acetonitrile are preferred) or by reacting the free base or acid with an excess of the desired salt-forming inorganic or organic acid or base in a suitable solvent or various combinations of solvents. Lists of suitable salts are found in Remington's Pharmaceutical Sciences, 17th ed., Mack Publishing Company, Easton, Pa., 1985, p. 1418, et al., the entire disclosure of which is incorporated herein by reference.

Further, exemplary acid addition salts include acetates, ascorbates, benzoates, benzenesulfonates, bisulfates, borates, butyrates, citrates, camphorates, camphorsulfonates, fumarates, hydrochlorides, hydrobromides, hydroiodides, lactates, maleates, methanesulfonates, naphthalenesulfonates, nitrates, oxalates, phosphates, propionates, salicylates, succinates, sulfates, tartarates, thiocyanates, toluenesulfonates (also known as tosylates) and the like. Additionally, acids which are generally considered suitable for the formation of pharmaceutically useful salts from basic pharmaceutical compounds are discussed, for example, by S. Berge et al, Journal of Pharmaceutical Sciences (1977) 66(1) 1-19; P. Gould, International J. of Pharmaceutics (1986) 33 201-217; Anderson et al, The Practice of Medicinal Chemistry (1996), Academic Press, New York; and in The Orange Book (Food & Drug Administration, Washington, D.C. on their website). These disclosures are incorporated herein by reference thereto.

Exemplary basic salts include ammonium salts, alkali metal salts such as sodium, lithium, and potassium salts, alkaline earth metal salts such as calcium and magnesium salts, salts with organic bases (for example, organic amines) such as dicyclohexylamines, t-butyl amines, and salts with amino acids such as arginine, lysine and the like. Basic nitrogen-containing groups may be quarternized with agents such as lower alkyl halides (e.g. methyl, ethyl, and butyl chlorides, bromides and iodides), dialkyl sulfates (e.g. dimethyl, diethyl, and dibutyl sulfates), long chain halides (e.g. decyl, lauryl, and stearyl chlorides, bromides and iodides), aralkyl halides (e.g. benzyl and phenethyl bromides), and others.

All such acid salts and base salts are intended to be pharmaceutically acceptable salts within the scope of the invention and all acid and base salts are considered equivalent to the free forms of the corresponding compounds for purposes of the invention.

“Pharmaceutically effective” or “therapeutically effective” amount of a compound of the present invention is an amount that is sufficient to effect the desired therapeutic, ameliorative, inhibitory or preventative effect, as defined herein, when administered to a mammal in need of such treatment. The amount will vary depending upon the subject and disease condition being treated, the weight and age of the subject, the severity of the disease condition, the manner of administration and the like, which can be readily determined by one of skill in the art.

“Mammal” as used herein means humans and other mammalian animals.

“Treatment” as used herein refers to any treatment of a disease (e.g., diabetes mellitus) or condition in a mammal, particularly a human, and includes, without limitation: (i) preventing the disease or condition from occurring in a subject which may be predisposed to the condition but has not yet been diagnosed with the condition and, accordingly, the treatment constitutes prophylactic treatment for the pathologic condition; (ii) inhibiting the disease or condition, i.e., arresting its development; (iii) relieving the disease or condition, i.e., causing regression of the disease or condition; or (iv) relieving the symptoms resulting from the disease or condition, e.g., relieving an inflammatory response without addressing the underlining disease or condition.

The present invention also envisions the quaternization of any basic nitrogen-containing groups of the compounds disclosed herein. The basic nitrogen can be quaternized with any agents known to those of ordinary skill in the art including, without limitation, lower alkyl halides, such as methyl, ethyl, propyl and butyl chlorides, bromides and iodides; dialkyl sulfates including dimethyl, diethyl, dibutyl and diamyl sulfates; long chain halides such as decyl, lauryl, myristyl and stearyl chlorides, bromides and iodides; and aralkyl halides including benzyl and phenethyl bromides. Water or oil-soluble or dispersible products may be obtained by such quaternization.

Without being bound by the above general structural descriptions/definitions, preferred compounds suitable as BRMs include, but are not limited to the following compounds. It will be appreciated, as noted above, that where an R or S enantiomer is exemplified for each particular compound, the corresponding S or R enantiomer, respectively, is also intended even though it may not be specifically shown below.

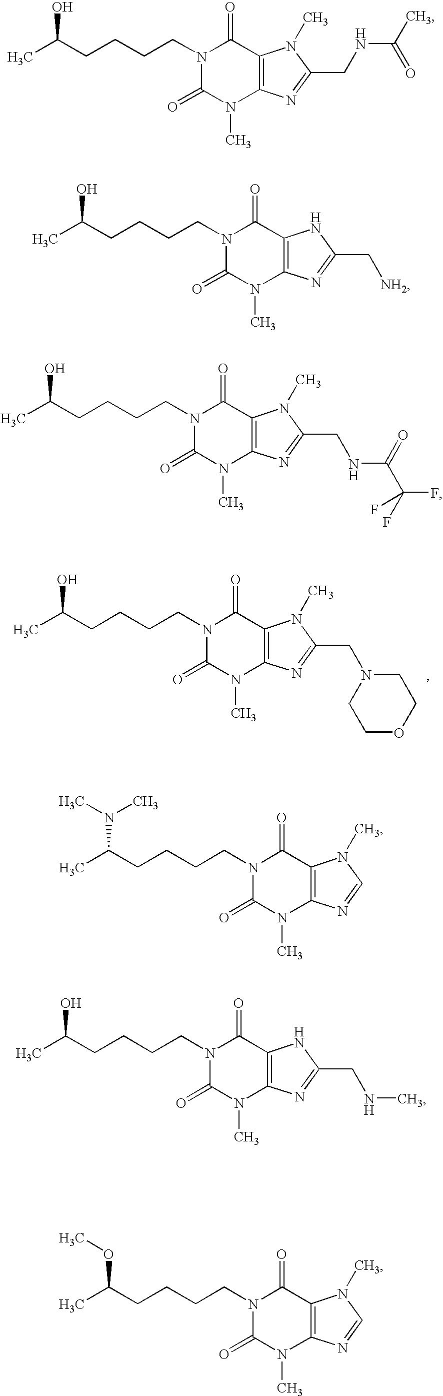

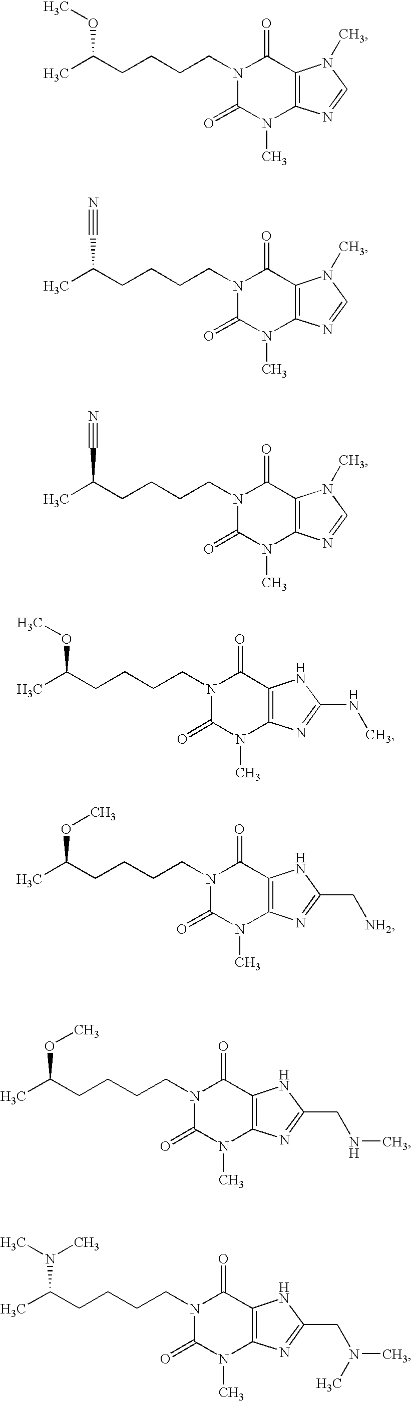

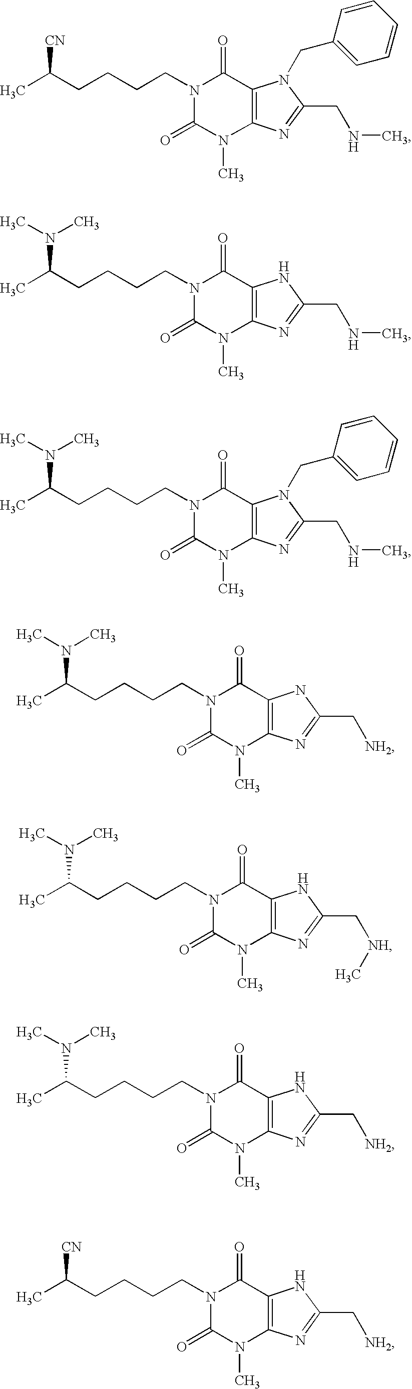

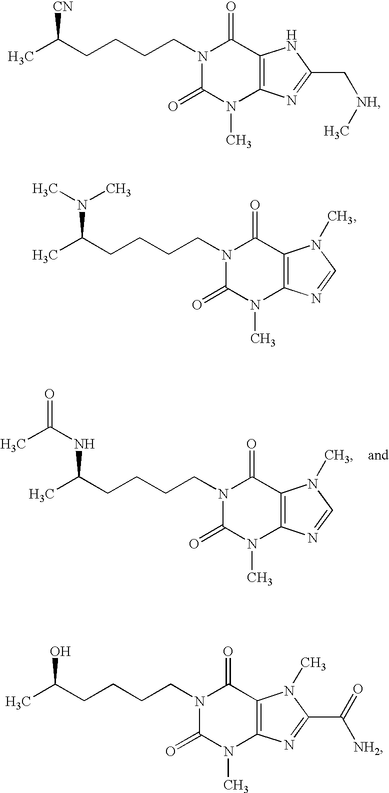

Further representative compounds of the present invention having utility as a biological/immune response modifier (immunomodulating) or anti-inflammatory agent in accordance with the present invention are set forth below in Table 1. The compounds in Table 1 have the following general structure of Formula II:

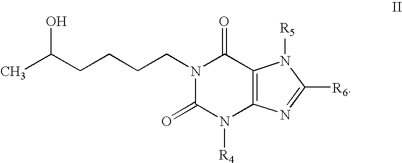

It is noted that in Table 1, “Me” represents “—CH3,” and “Et” represents “—CH2CH3.” In addition, although the below-exemplified moieties in Table 1 are representative of R4, R5 and R6 in Formula II, it will be understood that the exemplified moieties, without being limited by the above description/definitions, are also representative of R2 and R3 in Formula I.

TABLE 1

| R₄ | R₅ | R₆ |
|---|---|---|
| Me | H | 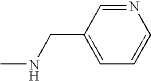 |
| Me | H | 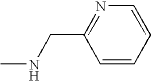 |
| Me | H | 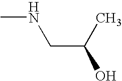 |
| Me | H | 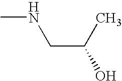 |
| Me | H | 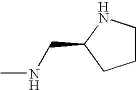 |
| Me | H | 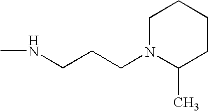 |
| Me | H | 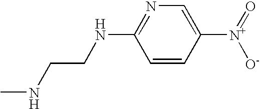 |
| Me | H | 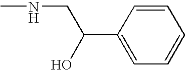 |
| Me | H | 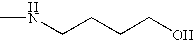 |
| Me | H | 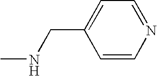 |
| Me | H | 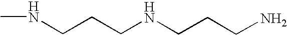 |
| Me | H | 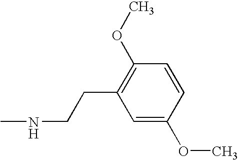 |
| Me | H | 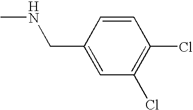 |
| Me | H | 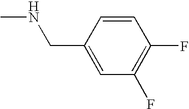 |
| Me | H | 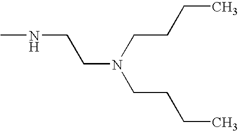 |
| Me | H | 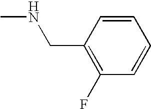 |
| Me | H | 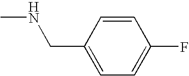 |
| Me | H | 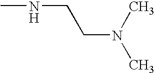 |
| Me | H | 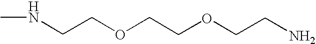 |
| Me | H | 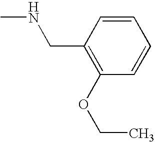 |
| Me | H | 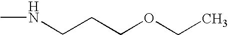 |
| Me | H | 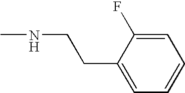 |
| Me | H | 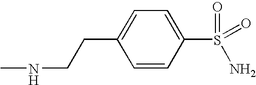 |
| Me | H | 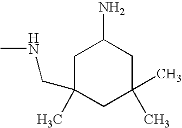 |
| Me | H | 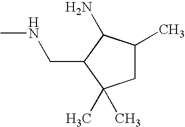 |
| Me | H | 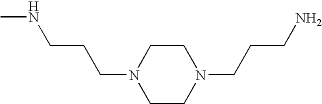 |
| Me | H | 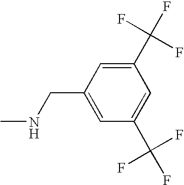 |
| Me | H | 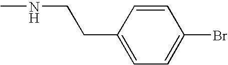 |
| Me | H | 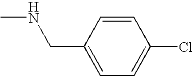 |
| Me | H | 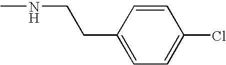 |
| Me | H | 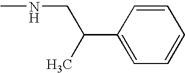 |
| Me | H | 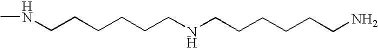 |
| Me | H | 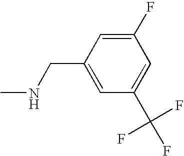 |
| Me | H | 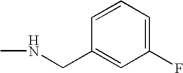 |
| Me | H | 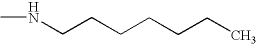 |
| Me | H | 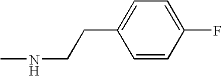 |
| Me | H | 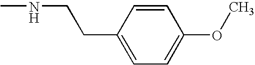 |
| Me | H | 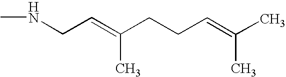 |
| Me | H | 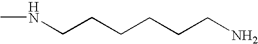 |
| Me | H | 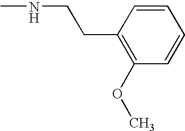 |
| Me | H | 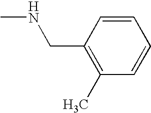 |
| Me | H | 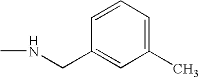 |
| Me | H | 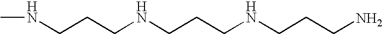 |
| Me | H | 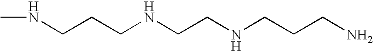 |
| Me | H | 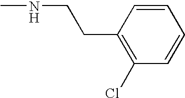 |
| Me | H | 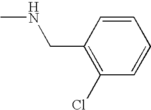 |
| Me | H | 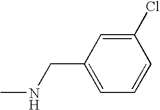 |
| Me | H | 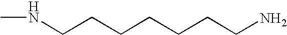 |
| Me | H | 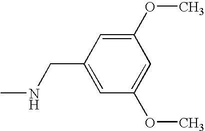 |
| Me | H | 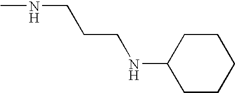 |
| Me | H | 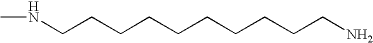 |
| Me | H | 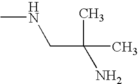 |
| Me | H | 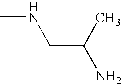 |
| Me | H | 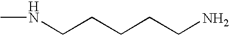 |
| Me | H | 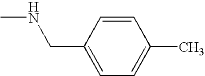 |
| Me | H | 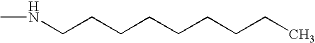 |
| Me | H | 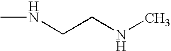 |
| Me | H | 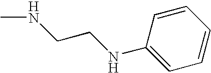 |
| Me | H | 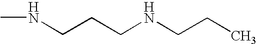 |
| Me | H | 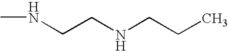 |
| Me | H | 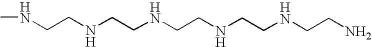 |
| Me | H | 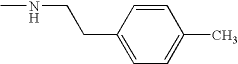 |
| Me | H | 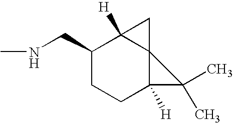 |
| Me | H | 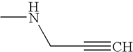 |
| Me | H | 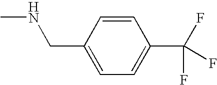 |
| Me | H | 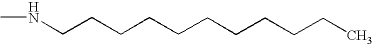 |
| Me | H | 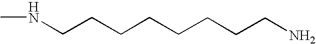 |
| Me | H | 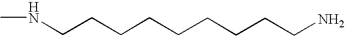 |
| Me | H | 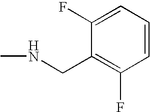 |
| Me | H | 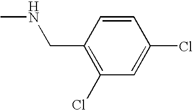 |
| Me | H | 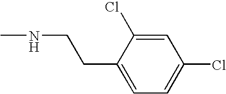 |
| Me | H | 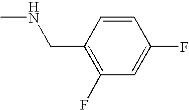 |
| Me | H | 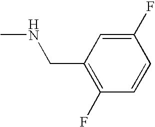 |
| Me | H | 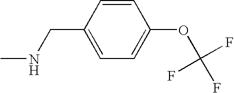 |
| Me | H | 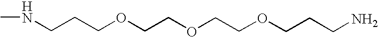 |
| Me | H | 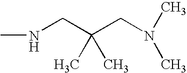 |
| Me | H | 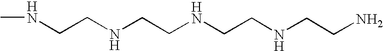 |
| Me | H | 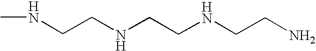 |
| Me | H | 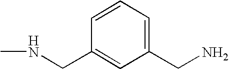 |
| Me | H | 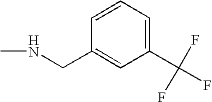 |
| Me | H | 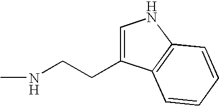 |
| Me | H | 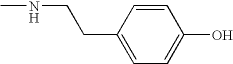 |
| Me | H | 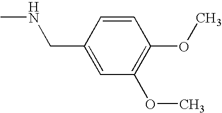 |
| Me | H | 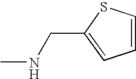 |
| Me | H | 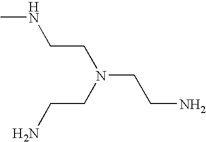 |
| Me | H | 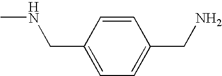 |
| Me | H | 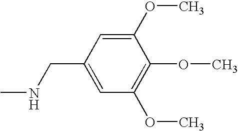 |
| Me | H | 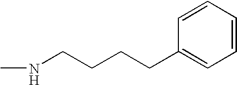 |
| Me | H | 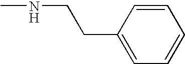 |
| Me | H | 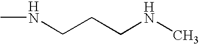 |
| Me | H | 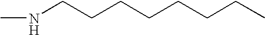 |
| Me | H | 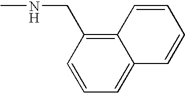 |
| Me | H | 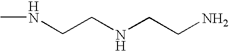 |
| Me | H | 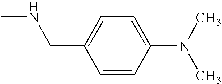 |
| Me | H | 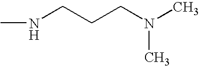 |
| Me | H | 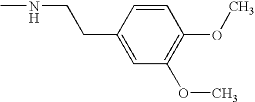 |
| Me | H | 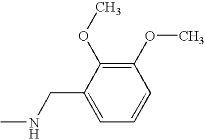 |
| Me | H | 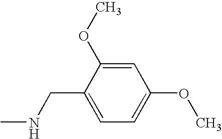 |
| Me | H | 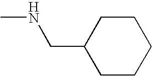 |
| Me | H | 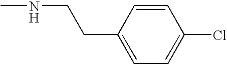 |
| Me | H | 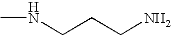 |
| Me | H | 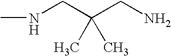 |
| Me | H | 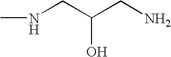 |
| Me | H | 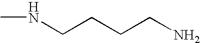 |
| Me | H | 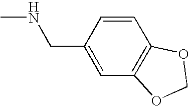 |
| Me | H | 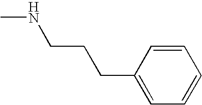 |
| Me | H | 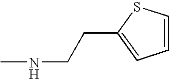 |
| Me | H | 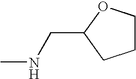 |
| Me | H | 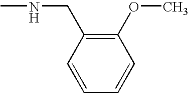 |
| Me | H | 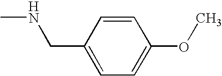 |
| Me | H | 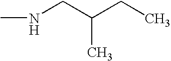 |
| Me | H | 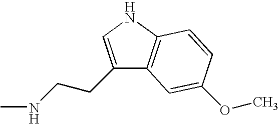 |
| Me | H | 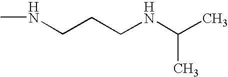 |
| Me | H | 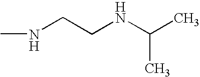 |
| Me | H | 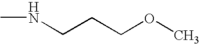 |
| Me | H | 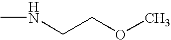 |
| Me | H | 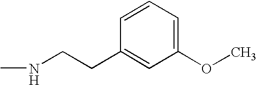 |
| Me | H | 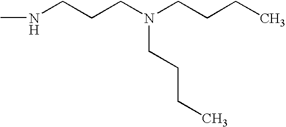 |
| Me | H | 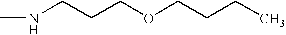 |
| Me | H | 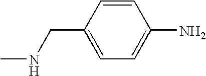 |
| Me | H | 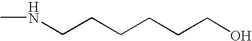 |
| Me | H | 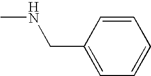 |
| Me | H | 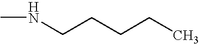 |
| Me | H | 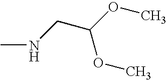 |
| Me | H | 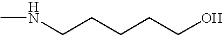 |
| Me | H | 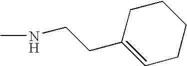 |
| Me | H | 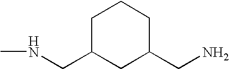 |
| Me | H | 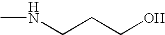 |
| Me | H | 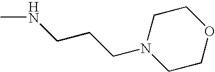 |
| Me | H | 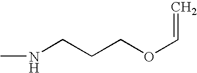 |
| Me | H | 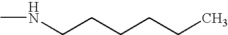 |
| Me | H | 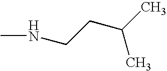 |
| Me | H | 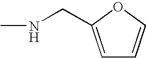 |
| Me | H | 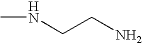 |
| Me | H | 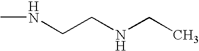 |
| Me | H | 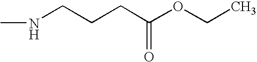 |
| Me | H | 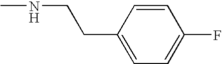 |
| Me | H | 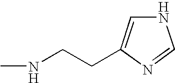 |
| Me | H | 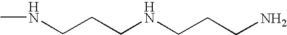 |
| Me | H | 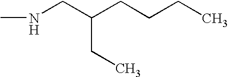 |
| Me | H | 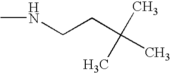 |
| Me | H | 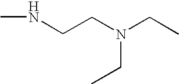 |
| Me | H | 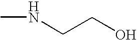 |
| Me | H | 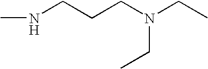 |
| Me | H | 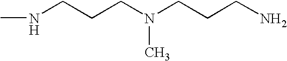 |
| Me | H | 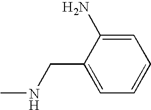 |
| Me | H | 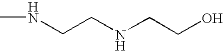 |
| Me | H | 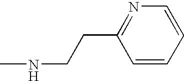 |
| Me | H | 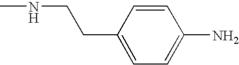 |
| Me | H | 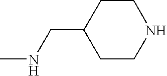 |
| Me | H | 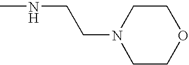 |
| Me | H | 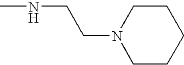 |
| Me | H | 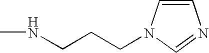 |
| Me | H | 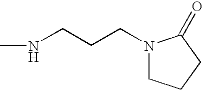 |
| Me | H | 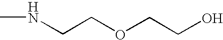 |
| Me | H | 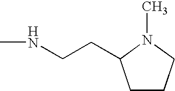 |
| Me | H | 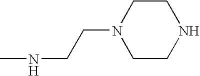 |
| Me | H | 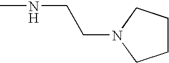 |
| Me | Me | 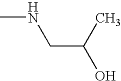 |
| Me | Me | 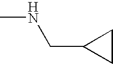 |
| Me | Me | 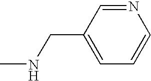 |
| Me | Me | 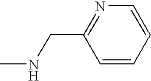 |
| Me | Me | 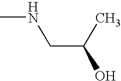 |
| Me | Me | 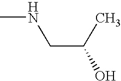 |
| Me | Me | 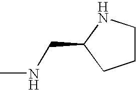 |
| Me | Me | 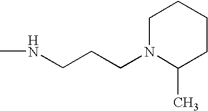 |
| Me | Me | 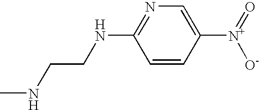 |
| Me | Me | 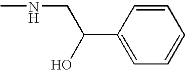 |
| Me | Me | 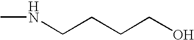 |
| Me | Me | 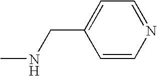 |
| Me | Me | 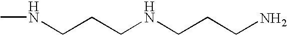 |
| Me | Me | 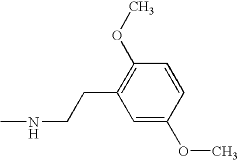 |
| Me | Me | 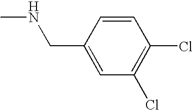 |
| Me | Me | 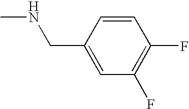 |
| Me | Me | 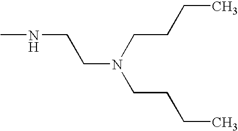 |
| Me | Me | 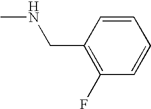 |
| Me | Me | 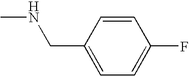 |
| Me | Me | 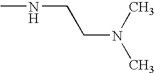 |
| Me | Me | 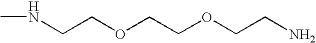 |
| Me | Me | 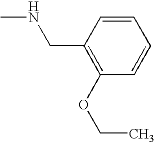 |
| Me | Me | 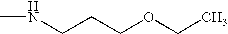 |
| Me | Me | 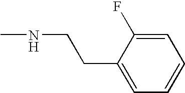 |
| Me | Me | 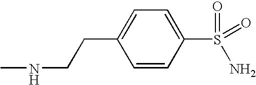 |
| Me | Me | 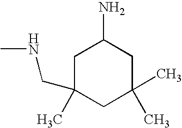 |
| Me | Me | 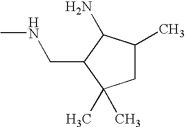 |
| Me | Me | 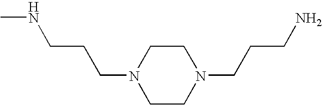 |
| Me | Me | 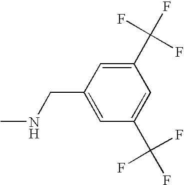 |
| Me | Me | 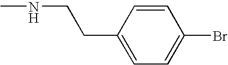 |
| Me | Me | 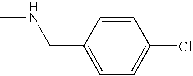 |
| Me | Me | 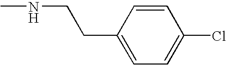 |
| Me | Me | 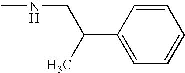 |
| Me | Me | 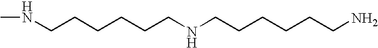 |
| Me | Me | 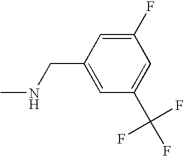 |
| Me | Me | 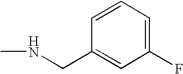 |
| Me | Me | 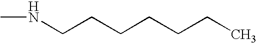 |
| Me | Me | 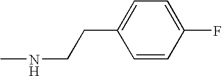 |
| Me | Me | 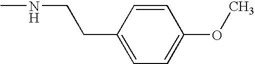 |
| Me | Me | 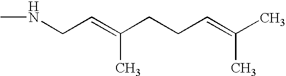 |
| Me | Me | 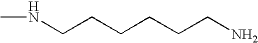 |
| Me | Me | 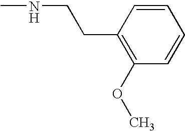 |
| Me | Me | 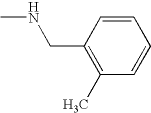 |
| Me | Me | 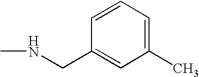 |
| Me | Me | 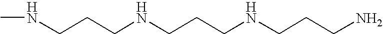 |
| Me | Me | 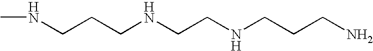 |
| Me | Me | 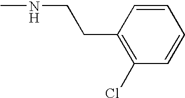 |
| Me | Me | 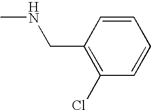 |
| Me | Me | 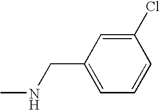 |
| Me | Me | 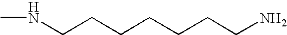 |
| Me | Me | 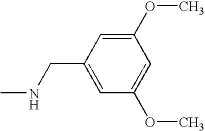 |
| Me | Me | 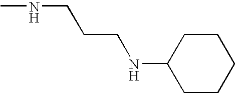 |
| Me | Me | 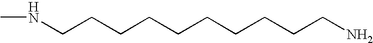 |
| Me | Me | 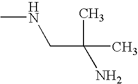 |
| Me | Me | 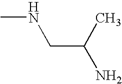 |
| Me | Me | 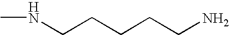 |
| Me | Me | 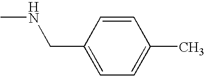 |
| Me | Me | 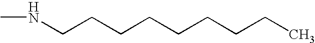 |
| Me | Me | 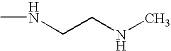 |
| Me | Me | 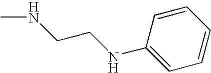 |
| Me | Me | 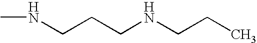 |
| Me | Me | 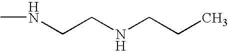 |
| Me | Me | 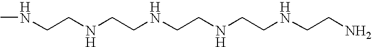 |
| Me | Me | 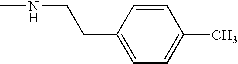 |
| Me | Me | 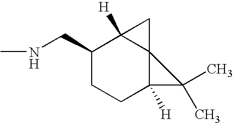 |
| Me | Me | 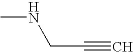 |
| Me | Me | 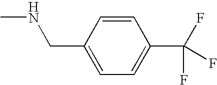 |
| Me | Me | 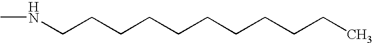 |
| Me | Me | 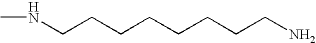 |
| Me | Me | 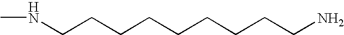 |
| Me | Me | 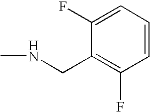 |
| Me | Me | 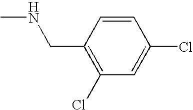 |
| Me | Me | 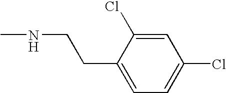 |
| Me | Me | 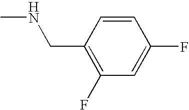 |
| Me | Me | 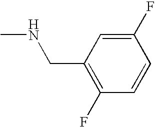 |
| Me | Me | 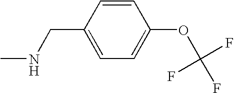 |
| Me | Me | 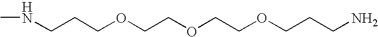 |
| Me | Me | 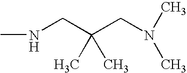 |
| Me | Me | 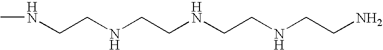 |
| Me | Me | 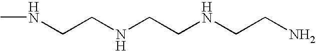 |
| Me | Me | 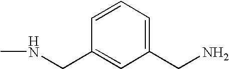 |
| Me | Me | 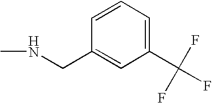 |
| Me | Me | 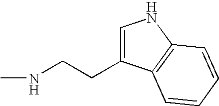 |
| Me | Me | 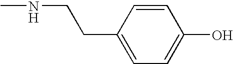 |
| Me | Me | 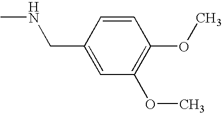 |
| Me | Me | 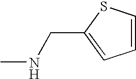 |
| Me | Me | 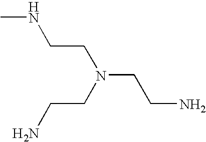 |
| Me | Me | 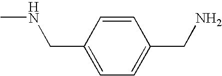 |
| Me | Me | 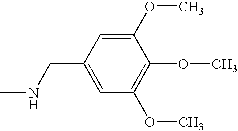 |
| Me | Me | 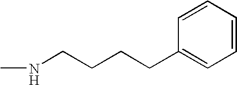 |
| Me | Me | 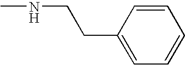 |
| Me | Me | 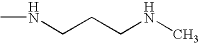 |
| Me | Me | 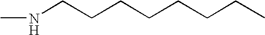 |
| Me | Me | 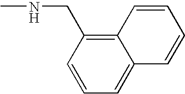 |
| Me | Me | 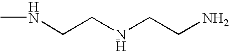 |
| Me | Me | 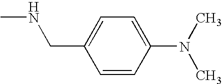 |
| Me | Me | 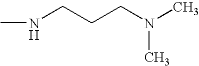 |
| Me | Me | 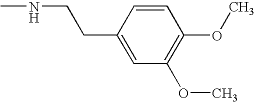 |
| Me | Me | 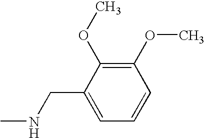 |
| Me | Me | 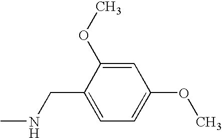 |
| Me | Me | 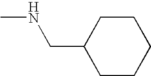 |
| Me | Me | 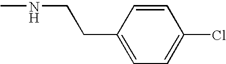 |
| Me | Me | 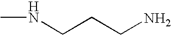 |
| Me | Me | 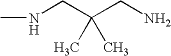 |
| Me | Me | 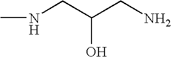 |
| Me | Me | 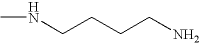 |
| Me | Me | 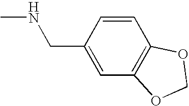 |
| Me | Me | 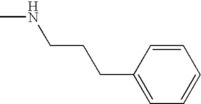 |
| Me | Me | 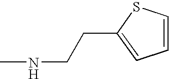 |
| Me | Me | 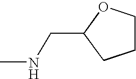 |
| Me | Me | 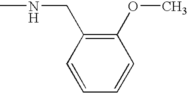 |
| Me | Me | 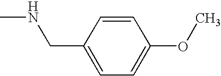 |
| Me | Me | 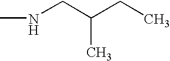 |
| Me | Me | 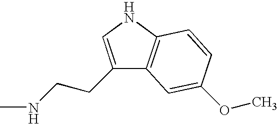 |
| Me | Me | 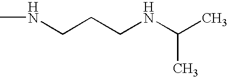 |
| Me | Me | 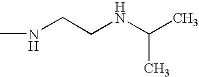 |
| Me | Me | 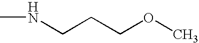 |
| Me | Me | 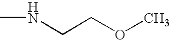 |
| Me | Me | 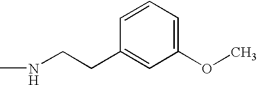 |
| Me | Me | 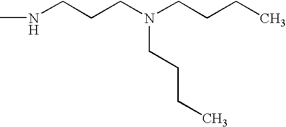 |
| Me | Me | 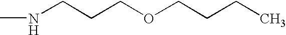 |
| Me | Me | 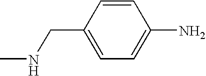 |
| Me | Me | 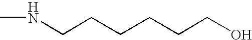 |
| Me | Me | 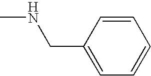 |
| Me | Me | 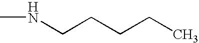 |
| Me | Me | 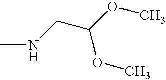 |
| Me | Me | 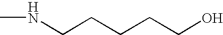 |
| Me | Me | 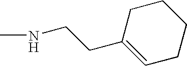 |
| Me | Me | 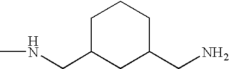 |
| Me | Me | 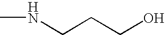 |
| Me | Me | 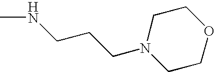 |
| Me | Me | 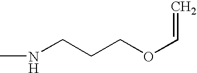 |
| Me | Me | 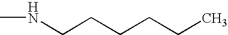 |
| Me | Me | 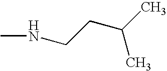 |
| Me | Me | 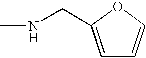 |
| Me | Me | 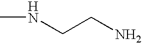 |
| Me | Me | 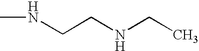 |
| Me | Me | 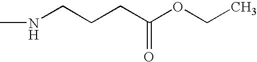 |
| Me | Me | 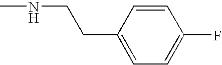 |
| Me | Me | 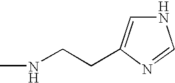 |
| Me | Me | 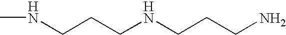 |
| Me | Me | 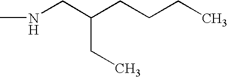 |
| Me | Me | 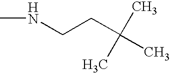 |
| Me | Me | 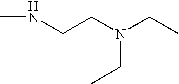 |
| Me | Me | 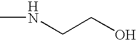 |
| Me | Me | 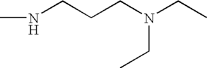 |
| Me | Me | 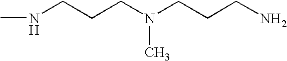 |
| Me | Me | 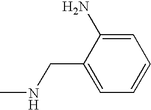 |
| Me | Me | 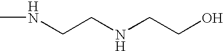 |
| Me | Me | 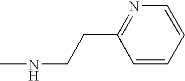 |
| Me | Me | 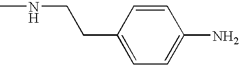 |
| Me | Me | 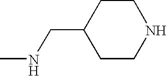 |
| Me | Me | 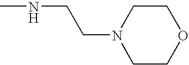 |
| Me | Me | 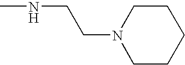 |
| Me | Me | 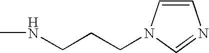 |
| Me | Me | 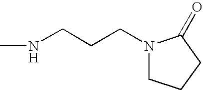 |
| Me | Me | 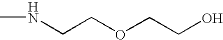 |
| Me | Me | 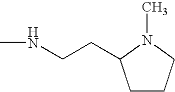 |
| Me | Me | 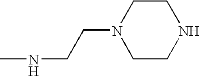 |
| Me | Me | 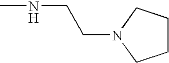 |
| Me | Me | 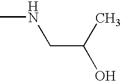 |
| Me | Me | 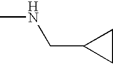 |
| Me | CH₂OEt | 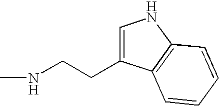 |
| Me | CH₂OEt | 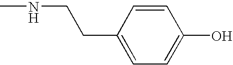 |
| Me | CH₂OEt | 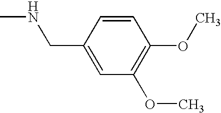 |
| Me | CH₂OEt | 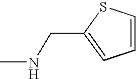 |
| Me | CH₂OEt | 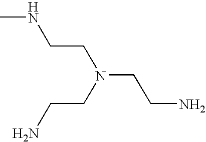 |
| Me | CH₂OEt | 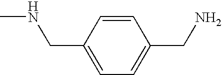 |
| Me | CH₂OEt | 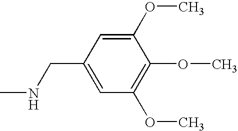 |
| Me | CH₂OEt | 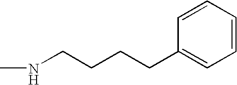 |
| Me | CH₂OEt | 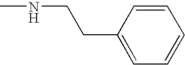 |
| Me | CH₂OEt | 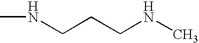 |
| Me | CH₂OEt | 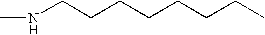 |
| Me | CH₂OEt | 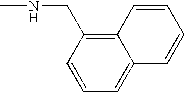 |
| Me | CH₂OEt | 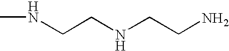 |
| Me | CH₂OEt | 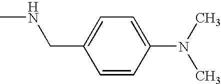 |
| Me | CH₂OEt | 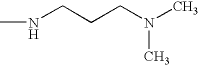 |
| Me | CH₂OEt | 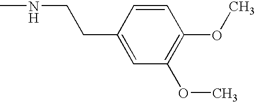 |
| Me | CH₂OEt | 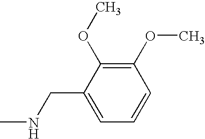 |
| Me | CH₂OEt | 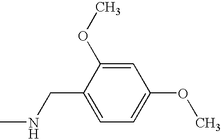 |
| Me | CH₂OEt | 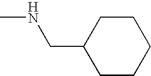 |
| Me | CH₂OEt | 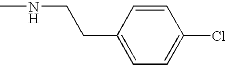 |
| Me | CH₂OEt | 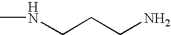 |
| Me | CH₂OEt | 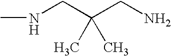 |
| Me | CH₂OEt | 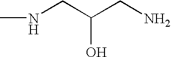 |
| Me | CH₂OEt | 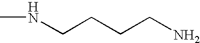 |
| Me | CH₂OEt | 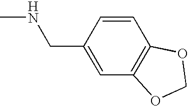 |
| Me | CH₂OEt | 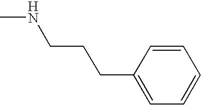 |
| Me | CH₂OEt | 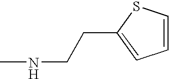 |
| Me | CH₂OEt | 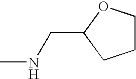 |
| Me | CH₂OEt | 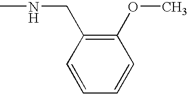 |
| Me | CH₂OEt | 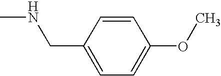 |
| Me | CH₂OEt | 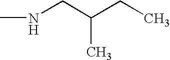 |
| Me | CH₂OEt | 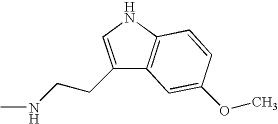 |
| Me | CH₂OEt | 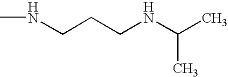 |
| Me | CH₂OEt | 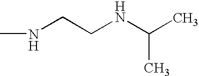 |
| Me | CH₂OEt | 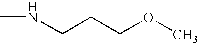 |
| Me | CH₂OEt | 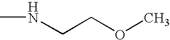 |
| Me | CH₂OEt | 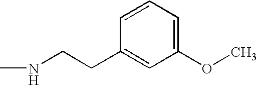 |
| Me | CH₂OEt | 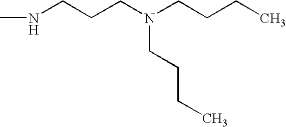 |
| Me | CH₂OEt | 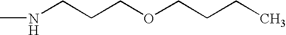 |
| Me | CH₂OEt | 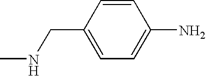 |
| Me | CH₂OEt | 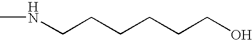 |
| Me | CH₂OEt | 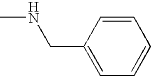 |
| Me | CH₂OEt | 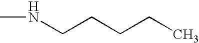 |
| Me | CH₂OEt | 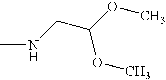 |
| Me | CH₂OEt | 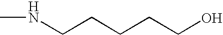 |
| Me | CH₂OEt | 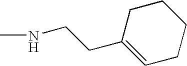 |
| Me | CH₂OEt | 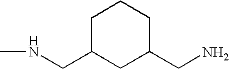 |
| Me | CH₂OEt | 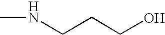 |
| Me | CH₂OEt | 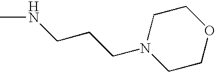 |
| Me | CH₂OEt | 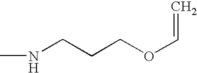 |
| Me | CH₂OEt | 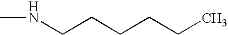 |
| Me | CH₂OEt | 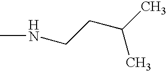 |
| Me | CH₂OEt | 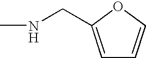 |
| Me | CH₂OEt | 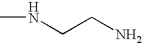 |
| Me | CH₂OEt | 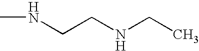 |
| Me | CH₂OEt | 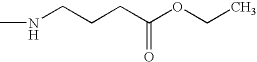 |
| Me | CH₂OEt | 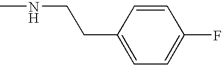 |
| Me | CH₂OEt | 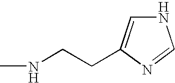 |
| Me | CH₂OEt | 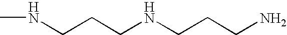 |
| Me | CH₂OEt | 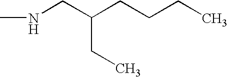 |
| Me | CH₂OEt | 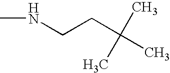 |
| Me | CH₂OEt | 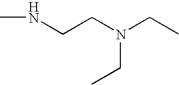 |
| Me | CH₂OEt | 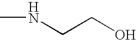 |
| Me | CH₂OEt | 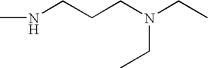 |
| Me | CH₂OEt | 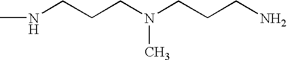 |
| Me | CH₂OEt | 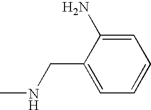 |
| Me | CH₂OEt | 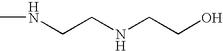 |
| Me | CH₂OEt | 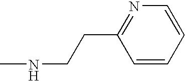 |
| Me | CH₂OEt | 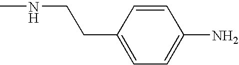 |
| Me | CH₂OEt | 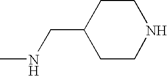 |
| Me | CH₂OEt | 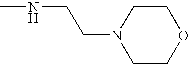 |
| Me | CH₂OEt | 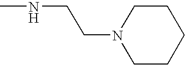 |
| Me | CH₂OEt | 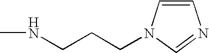 |
| Me | CH₂OEt | 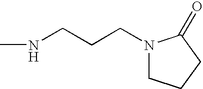 |
| Me | CH₂OEt | 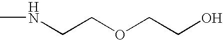 |
| Me | CH₂OEt | 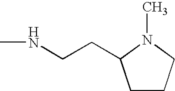 |
| Me | CH₂OEt | 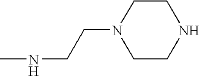 |
| Me | CH₂OEt | 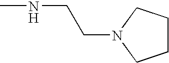 |
| Me | CH₂OEt | 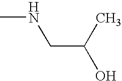 |
| Me | CH₂OEt | 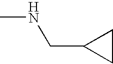 |
| Me | CH₂OEt | 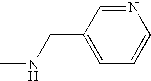 |
| Me | CH₂OEt | 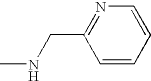 |
| Me | CH₂OEt | 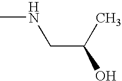 |
| Me | CH₂OEt | 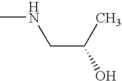 |
| Me | CH₂OEt | 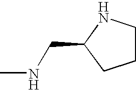 |
| Me | CH₂OEt | 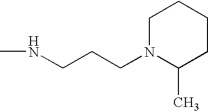 |
| Me | CH₂OEt | 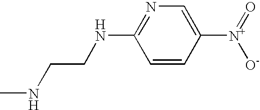 |
| Me | CH₂OEt | 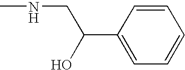 |
| Me | CH₂OEt | 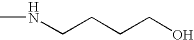 |
| Me | CH₂OEt | 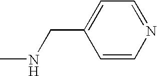 |
| Me | CH₂OEt | 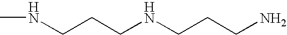 |
| Me | CH₂OEt | 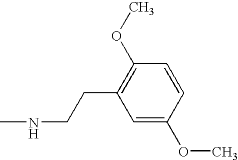 |
| Me | CH₂OEt | 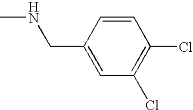 |
| Me | CH₂OEt | 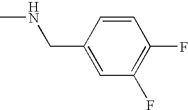 |
| Me | CH₂OEt | 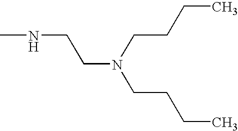 |
| Me | CH₂OEt | 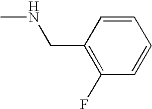 |
| Me | CH₂OEt | 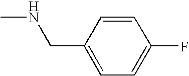 |
| Me | CH₂OEt | 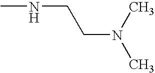 |
| Me | CH₂OEt | 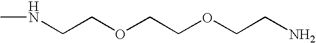 |
| Me | CH₂OEt | 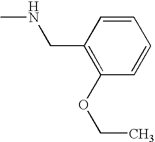 |
| Me | CH₂OEt | 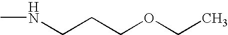 |
| Me | CH₂OEt | 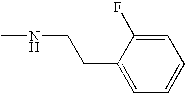 |
| Me | CH₂OEt | 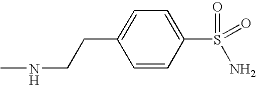 |
| Me | CH₂OEt | 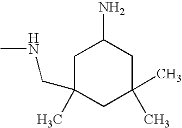 |
| Me | CH₂OEt | 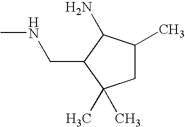 |
| Me | CH₂OEt | 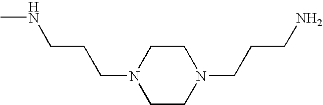 |
| Me | CH₂OEt | 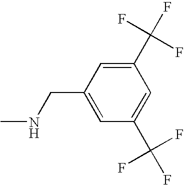 |
| Me | CH₂OEt | 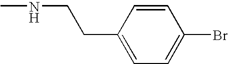 |
| Me | CH₂OEt | 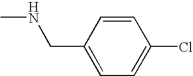 |
| Me | CH₂OEt | 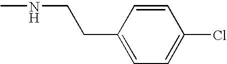 |
| Me | CH₂OEt | 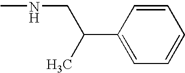 |
| Me | CH₂OEt | 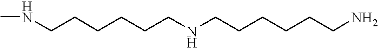 |
| Me | CH₂OEt | 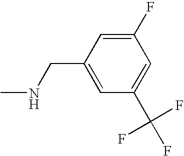 |
| Me | CH₂OEt | 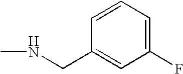 |
| Me | CH₂OEt | 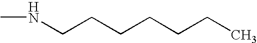 |
| Me | CH₂OEt | 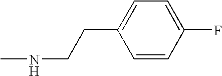 |
| Me | CH₂OEt | 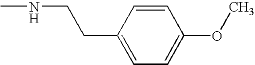 |
| Me | CH₂OEt | 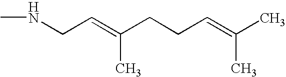 |
| Me | CH₂OEt | 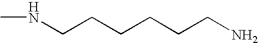 |
| Me | CH₂OEt | 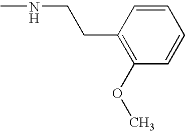 |
| Me | CH₂OEt | 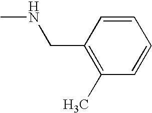 |
| Me | CH₂OEt | 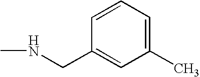 |
| Me | CH₂OEt | 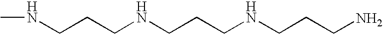 |
| Me | CH₂OEt | 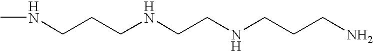 |
| Me | CH₂OEt | 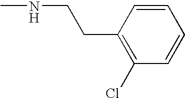 |
| Me | CH₂OEt | 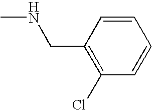 |
| Me | CH₂OEt | 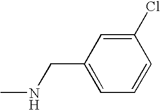 |
| Me | CH₂OEt | 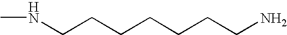 |
| Me | CH₂OEt | 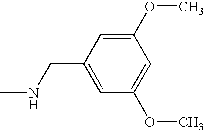 |
| Me | CH₂OEt | 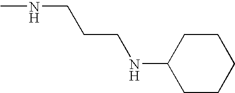 |
| Me | CH₂OEt | 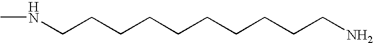 |
| Me | CH₂OEt | 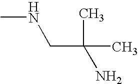 |
| Me | CH₂OEt | 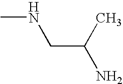 |
| Me | CH₂OEt | 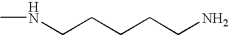 |
| Me | CH₂OEt | 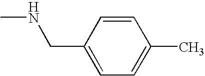 |
| Me | CH₂OEt | 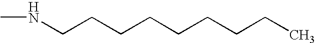 |
| Me | CH₂OEt | 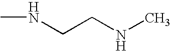 |
| Me | CH₂OEt | 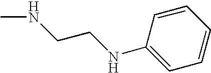 |
| Me | CH₂OEt | 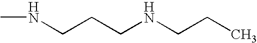 |
| Me | CH₂OEt | 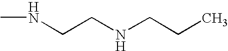 |
| Me | CH₂OEt | 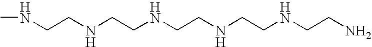 |
| Me | CH₂OEt | 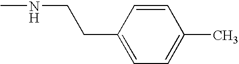 |
| Me | CH₂OEt | 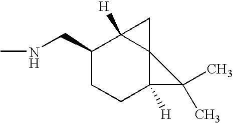 |
| Me | CH₂OEt | 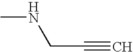 |
| Me | CH₂OEt | 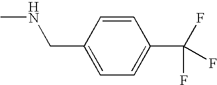 |
| Me | CH₂OEt | 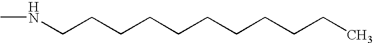 |
| Me | CH₂OEt | 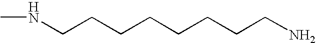 |
| Me | CH₂OEt | 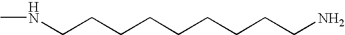 |
| Me | CH₂OEt | 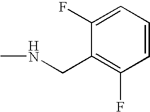 |
| Me | CH₂OEt | 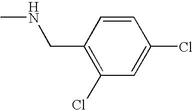 |
| Me | CH₂OEt | 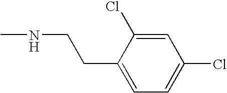 |
| Me | CH₂OEt | 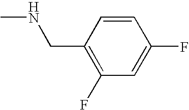 |
| Me | CH₂OEt | 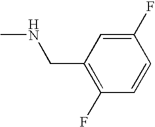 |
| Me | CH₂OEt | 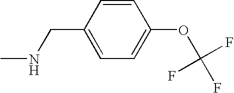 |
| Me | CH₂OEt | 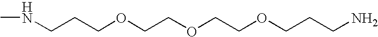 |
| Me | CH₂OEt | 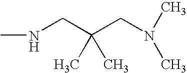 |
| Me | CH₂OEt | 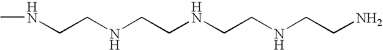 |
| Me | CH₂OEt | 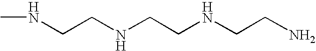 |
| Me | CH₂OEt | 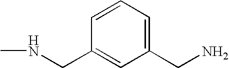 |
| Me | CH₂OEt | 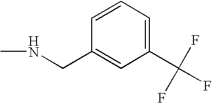 |
| 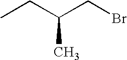 | Me | H |
| 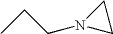 | Me | H |
| 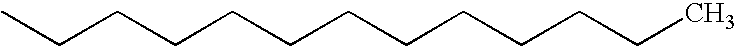 | Me | H |
| 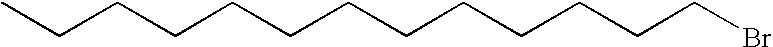 | Me | H |
| 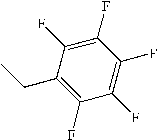 | Me | H |
| 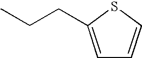 | Me | H |
| 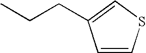 | Me | H |
| 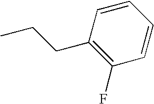 | Me | H |
| 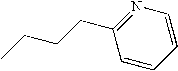 | Me | H |
| 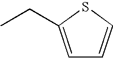 | Me | H |
| 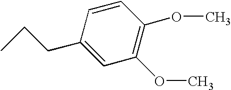 | Me | H |
| 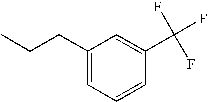 | Me | H |
| 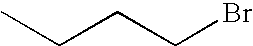 | Me | H |
| 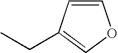 | Me | H |
| 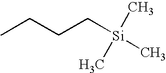 | Me | H |
| 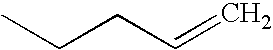 | Me | H |
| 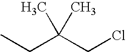 | Me | H |
| 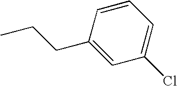 | Me | H |
| 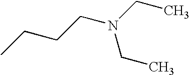 | Me | H |
| 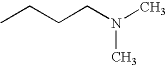 | Me | H |
| 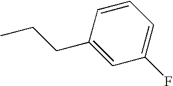 | Me | H |
| 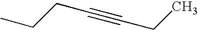 | Me | H |
| 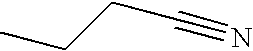 | Me | H |
| 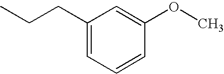 | Me | H |
| 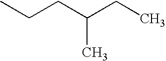 | Me | H |
| 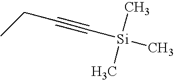 | Me | H |
| 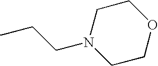 | Me | H |
| 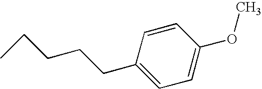 | Me | H |
| 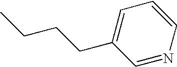 | Me | H |
| 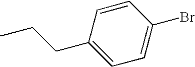 | Me | H |
| 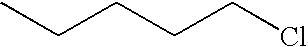 | Me | H |
| 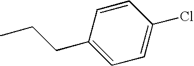 | Me | H |
| 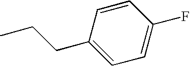 | Me | H |
| 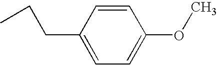 | Me | H |
| 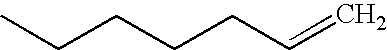 | Me | H |
| 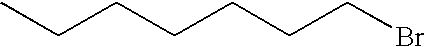 | Me | H |
| 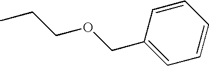 | Me | H |
| 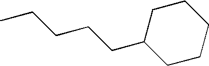 | Me | H |
| 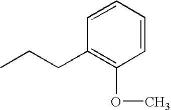 | Me | H |
| 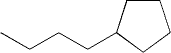 | Me | H |
| 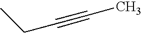 | Me | H |
| 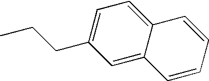 | Me | H |
| 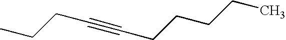 | Me | H |
| 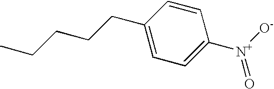 | Me | H |
| 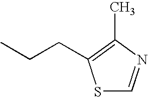 | Me | H |
| 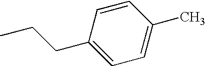 | Me | H |
| 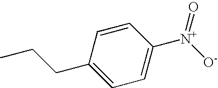 | Me | H |
| 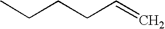 | Me | H |
| 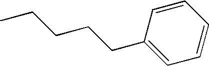 | Me | H |
| 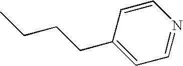 | Me | H |
| 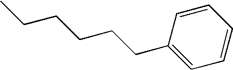 | Me | H |
| 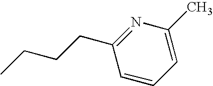 | Me | H |
| 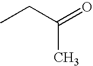 | Me | H |
| 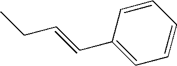 | Me | H |
| 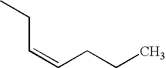 | Me | H |
| 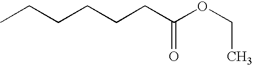 | Me | H |
| 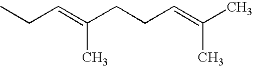 | Me | H |
| 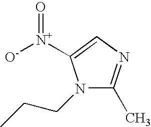 | Me | H |
| 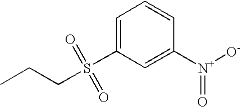 | Me | H |
| 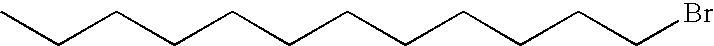 | Me | H |
| 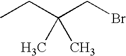 | Me | H |
| 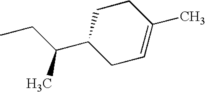 | Me | H |
| 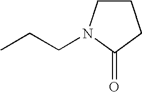 | Me | H |
| 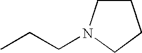 | Me | H |
| 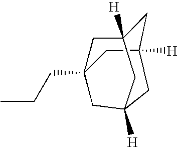 | Me | H |
| 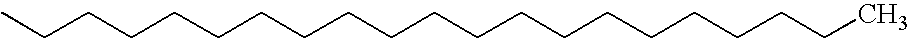 | Me | H |
| 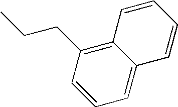 | Me | H |
| 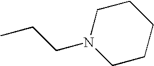 | Me | H |
| 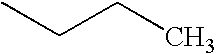 | Me | H |
| 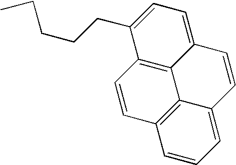 | Me | H |
| 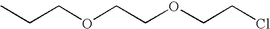 | Me | H |
| 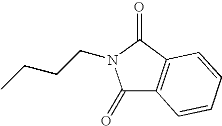 | Me | H |
| 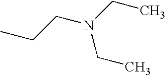 | Me | H |
| 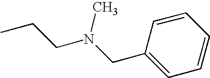 | Me | H |
| 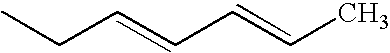 | Me | H |
| 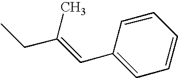 | Me | H |
| 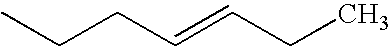 | Me | H |
| 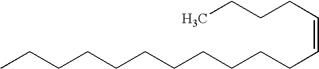 | Me | H |
| 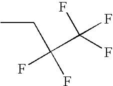 | Me | H |
| 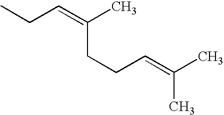 | Me | H |
| 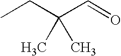 | Me | H |
| 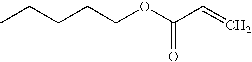 | Me | H |
| 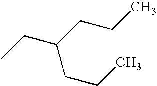 | Me | H |
| 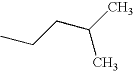 | Me | H |
| 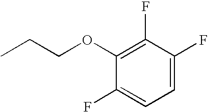 | Me | H |
| 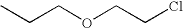 | Me | H |
| 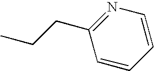 | Me | H |
| 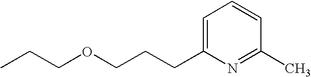 | Me | H |
| 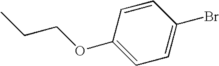 | Me | H |
| 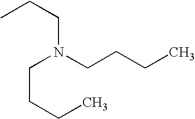 | Me | H |
| 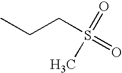 | Me | H |
| 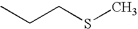 | Me | H |
| 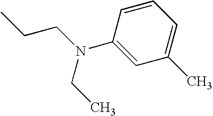 | Me | H |
| 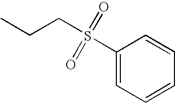 | Me | H |
| 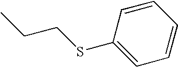 | Me | H |
| 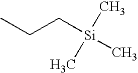 | Me | H |
| 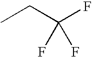 | Me | H |
| 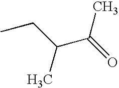 | Me | H |
| 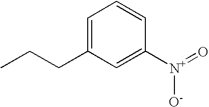 | Me | H |
| 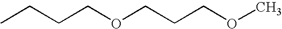 | Me | H |
| 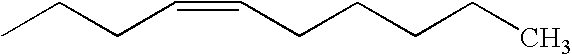 | Me | H |
| 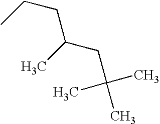 | Me | H |
| 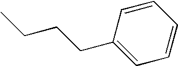 | Me | H |
| 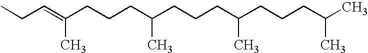 | Me | H |
| 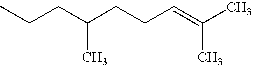 | Me | H |
| 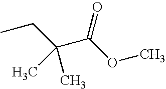 | Me | H |
| 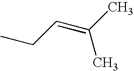 | Me | H |
| 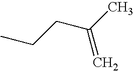 | Me | H |
| 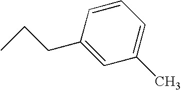 | Me | H |
| 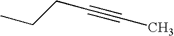 | Me | H |
| 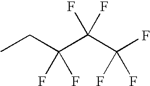 | Me | H |
| 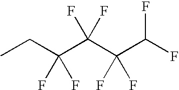 | Me | H |
| 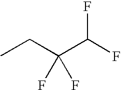 | Me | H |
| 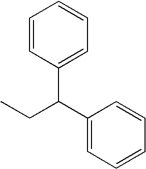 | Me | H |
| 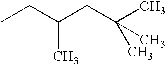 | Me | H |
| 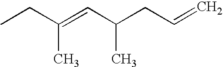 | Me | H |
| 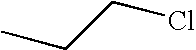 | Me | H |
| 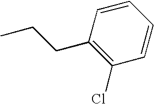 | Me | H |
| 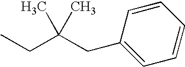 | Me | H |
| 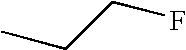 | Me | H |
| 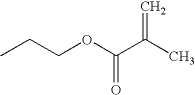 | Me | H |
| 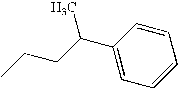 | Me | H |
| 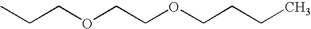 | Me | H |
| 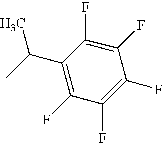 | Me | H |
| 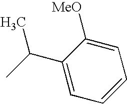 | Me | H |
| 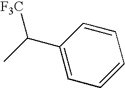 | Me | H |
| 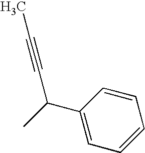 | Me | H |
| 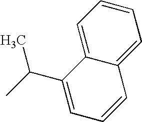 | Me | H |
| 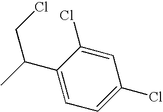 | Me | H |
| 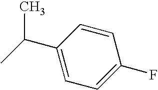 | Me | H |
| 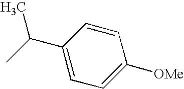 | Me | H |
| 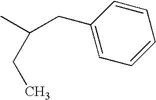 | Me | H |
| 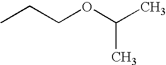 | Me | H |
| 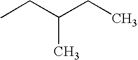 | Me | H |
| 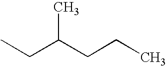 | Me | H |
| 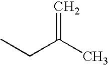 | Me | H |
| 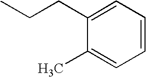 | Me | H |
| 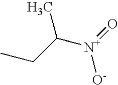 | Me | H |
| 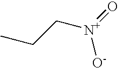 | Me | H |
| 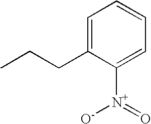 | Me | H |
| 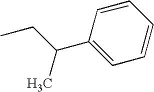 | Me | H |
| 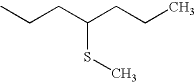 | Me | H |
| 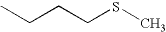 | Me | H |
| 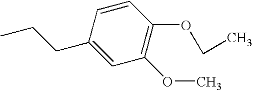 | Me | H |
| 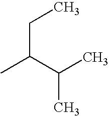 | Me | H |
| 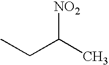 | Me | H |
| 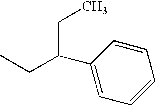 | Me | H |
| 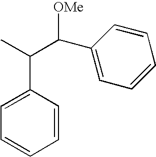 | Me | H |
| 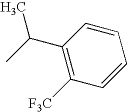 | Me | H |
| 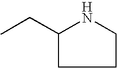 | Me | H |
| 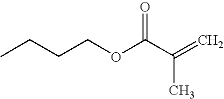 | Me | H |
| 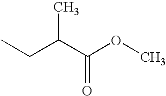 | Me | H |
| 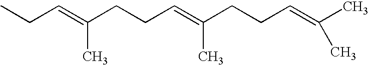 | Me | H |
| 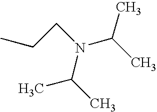 | Me | H |
| 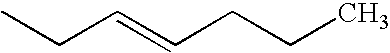 | Me | H |
| Me | 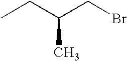 | H |
| Me | 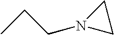 | H |
| Me | 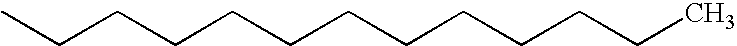 | H |
| Me | 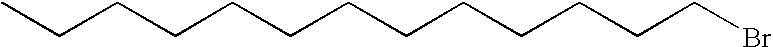 | H |
| Me | 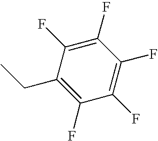 | H |
| Me | 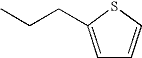 | H |
| Me | 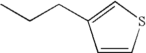 | H |
| Me | 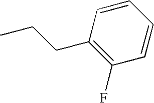 | H |
| Me | 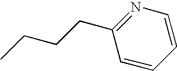 | H |
| Me | 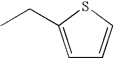 | H |
| Me | 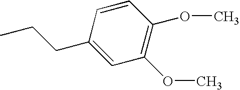 | H |
| Me | 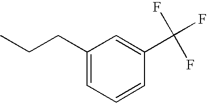 | H |
| Me | 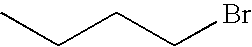 | H |
| Me | 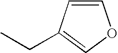 | H |
| Me | 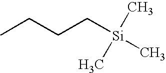 | H |
| Me | 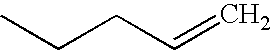 | H |
| Me | 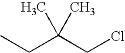 | H |
| Me | 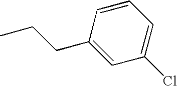 | H |
| Me | 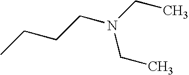 | H |
| Me | 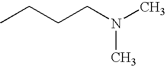 | H |
| Me | 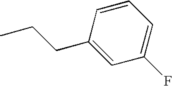 | H |
| Me | 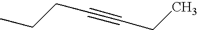 | H |
| Me | 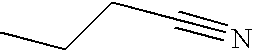 | H |
| Me | 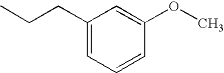 | H |
| Me | 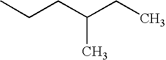 | H |
| Me | 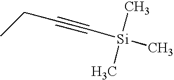 | H |
| Me | 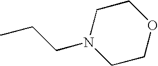 | H |
| Me | 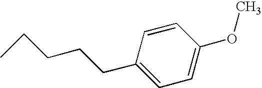 | H |
| Me | 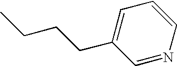 | H |
| Me | 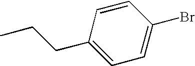 | H |
| Me | 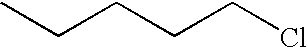 | H |
| Me | 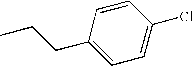 | H |
| Me | 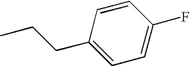 | H |
| Me | 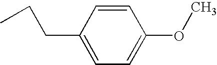 | H |
| Me | 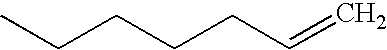 | H |
| Me | 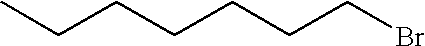 | H |
| Me | 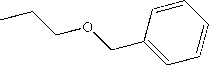 | H |
| Me | 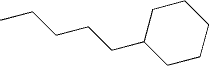 | H |
| Me | 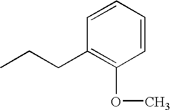 | H |
| Me | 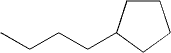 | H |
| Me | 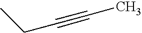 | H |
| Me | 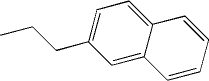 | H |
| Me | 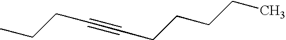 | H |
| Me | 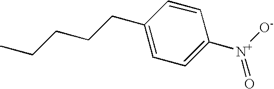 | H |
| Me | 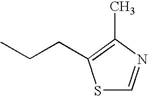 | H |
| Me | 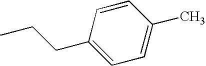 | H |
| Me | 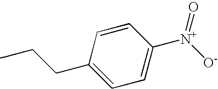 | H |
| Me | 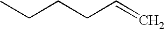 | H |
| Me | 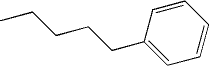 | H |
| Me | 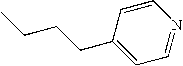 | H |
| Me | 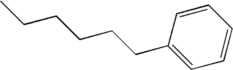 | H |
| Me | 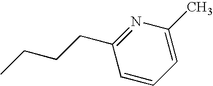 | H |
| Me | 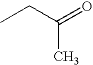 | H |
| Me | 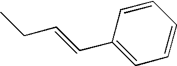 | H |
| Me | 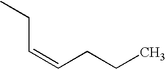 | H |
| Me | 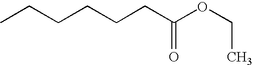 | H |
| Me | 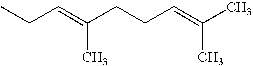 | H |
| Me | 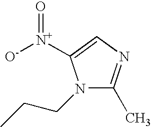 | H |
| Me | 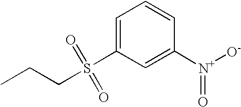 | H |
| Me | 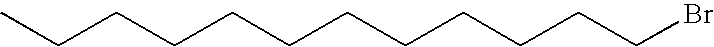 | H |
| Me | 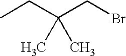 | H |
| Me | 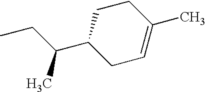 | H |
| Me | 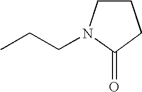 | H |
| Me | 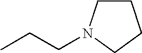 | H |
| Me | 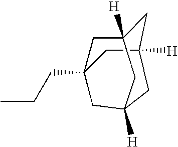 | H |
| Me | 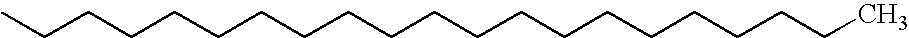 | H |
| Me | 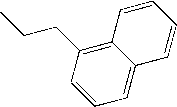 | H |
| Me | 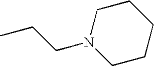 | H |
| Me | 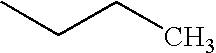 | H |
| Me | 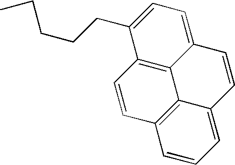 | H |
| Me | 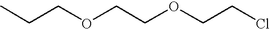 | H |
| Me | 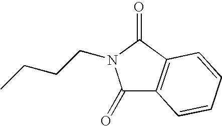 | H |
| Me | 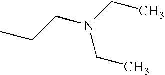 | H |
| Me | 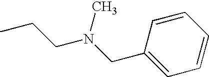 | H |
| Me | 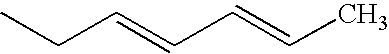 | H |
| Me | 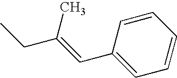 | H |
| Me | 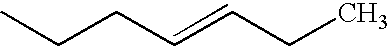 | H |
| Me | 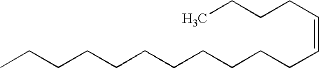 | H |
| Me | 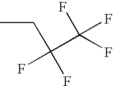 | H |
| Me | 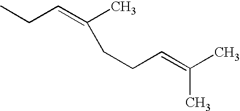 | H |
| Me | 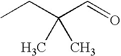 | H |
| Me | 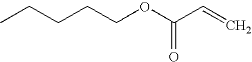 | H |
| Me | 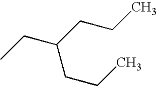 | H |
| Me | 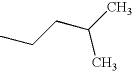 | H |
| Me | 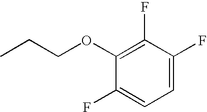 | H |
| Me | 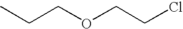 | H |
| Me | 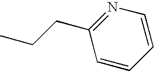 | H |
| Me | 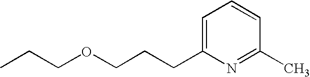 | H |
| Me | 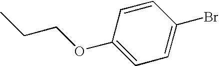 | H |
| Me | 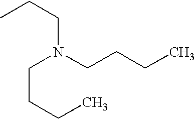 | H |
| Me | 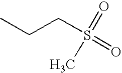 | H |
| Me | 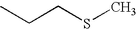 | H |
| Me | 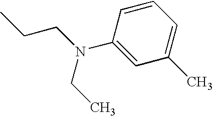 | H |
| Me | 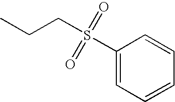 | H |
| Me | 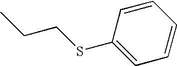 | H |
| Me | 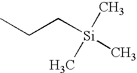 | H |
| Me | 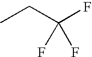 | H |
| Me | 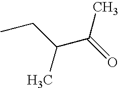 | H |
| Me | 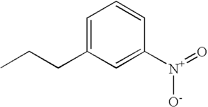 | H |
| Me | 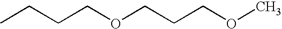 | H |
| Me | 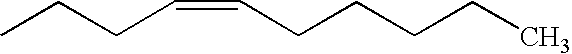 | H |
| Me | 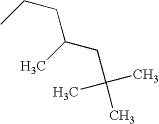 | H |
| Me | 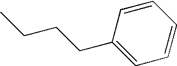 | H |
| Me | 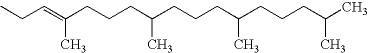 | H |
| Me | 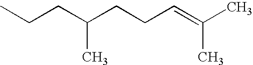 | H |
| Me | 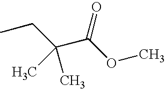 | H |
| Me | 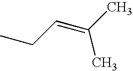 | H |
| Me | 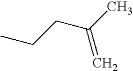 | H |
| Me | 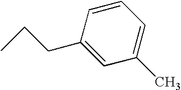 | H |
| Me | 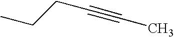 | H |
| Me | 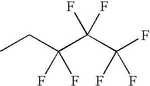 | H |
| Me | 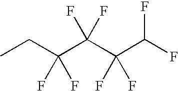 | H |
| Me | 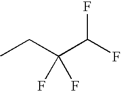 | H |
| Me | 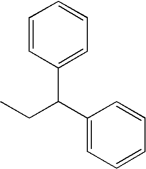 | H |
| Me | 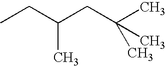 | H |
| Me | 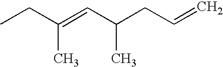 | H |
| Me | 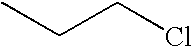 | H |
| Me | 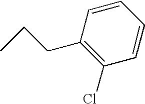 | H |
| Me | 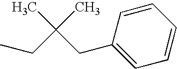 | H |
| Me | 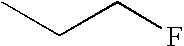 | H |
| Me | 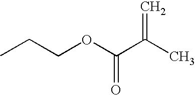 | H |
| Me | 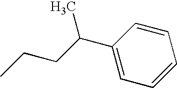 | H |
| Me | 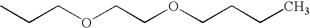 | H |
| Me | 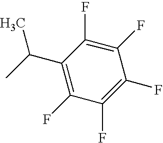 | H |
| Me | 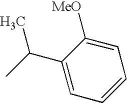 | H |
| Me | 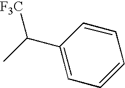 | H |
| Me | 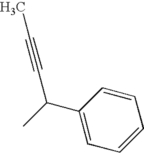 | H |
| Me | 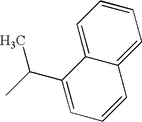 | H |
| Me | 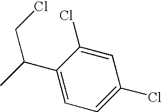 | H |
| Me | 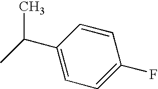 | H |
| Me | 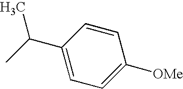 | H |
| Me | 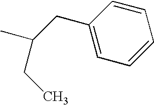 | H |
| Me | 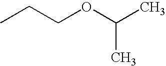 | H |
| Me | 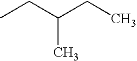 | H |
| Me | 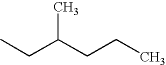 | H |
| Me | 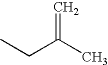 | H |
| Me | 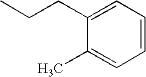 | H |
| Me | 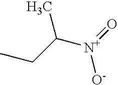 | H |
| Me | 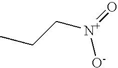 | H |
| Me | 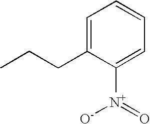 | H |
| Me | 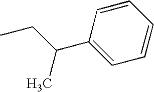 | H |
| Me | 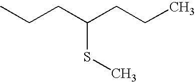 | H |
| Me | 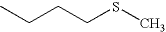 | H |
| Me | 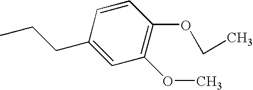 | H |
| Me | 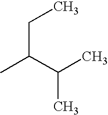 | H |
| Me | 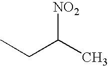 | H |
| Me | 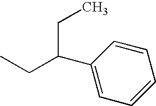 | H |
| Me | 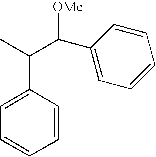 | H |
| Me | 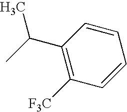 | H |
| Me | 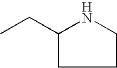 | H |
| Me | 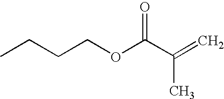 | H |
| Me | 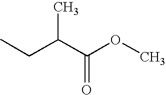 | H |
| Me | 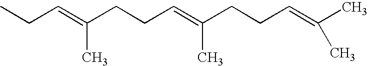 | H |
| Me | 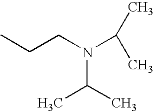 | H |
| Me | 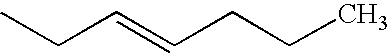 | H |

In addition to LSF, and the above-described LSF analogs, additional BRMs preferred for use in accordance with the principles of the present invention include, without limitation, members of the group consisting of the compounds (LSF analogs) described in the following U.S. patents, the entire disclosures or which are incorporated herein by reference:

| Pat. No. | Title |
|---|---|
| 5,585,380 | Modulation of Cellular Response to External Stimuli |
| 5,648,357 | Enantiomerically Pure Hydroxylated Xanthine Compounds |
| 5,652,243 | Methods of Using Enantiomericallly Pure Hydroxylated Xanthine Compounds |
| 5,612,349 | Enantiomerically Pure Hydroxylated Xanthine Compounds |
| 5,567,704 | R-Enantiomerically Pure Hydroxylated Xanthine Compounds To Treat |
| | Baldness |
| 5,580,874 | Enantiomerically Pure Hydroxylated Xanthine Compounds |
| 5,739,138 | Enantiomerically Pure Hydroxylated Xanthine Compounds To Treat |
| | Autoimmune Diabetes |
| 5,792,772 | Enantiomerically Pure Hydroxylated Xanthine Compounds |
| 5,620,984 | Enantiomerically Pure Hydroxylated Xanthine Compounds |
| 5,580,873 | Enantiomerically Pure Hydroxylated Xanthine Compounds To Treat |
| | Proliferative Vascular Diseases |
| 5,629,315 | Treatment of Diseases Using Enantiomerically Pure Hydroxylated Xanthine |
| | Compounds |
| 5,621,102 | Process for Preparing Enantiomerically Pure Xanthine Derivatives |
| 5,965,564 | Enantiomerically Pure Hydroxylated Xanthine Compounds |
| 5,629,423 | Asymmetric Synthesis of Chiral Secondary Alcohols |
| 6,780,865 | Compounds Having Selective Hydrolytic Potentials |
| 6,057,328 | Method for Treating Hyperoxia |
| 6,469,017 | Method of Inhibiting Interleukin-12 Signaling |
| 5,288,721 | Substituted Epoxyalkyl Xanthines for Modulation of Cellular Response |
| 5,866,576 | Expoxide - Containing Compounds |
| 6,121,270 | Epoxide - Containing Compounds |
| 5,340,813 | Substituted Aminoalkyl Xanthines Compounds |
| 5,817,662 | Substituted Amino Alkyl Compounds |
| 5,889,011 | Substituted Amino Alkyl Compounds |
| 6,103,730 | Amine Substituted Compounds |
| 5,801,182 | Amine Substituted Compounds |
| 5,807,861 | Amine Substituted Compounds |
| 5,473,070 | Substituted Long Chain Alcohol Xanthine Compounds |
| 5,804,584 | Hydroxyl-Containing Compounds |
| 5,780,476 | Hydroxyl-Containing Compounds |
| 6,133,274 | Hydroxyl-Containing Bicyclic Compounds |
| 6,693,105 | Hydroxyl-Containing Compounds |
| 6,075,029 | Modulators of Metabolism |
| 5,670,506 | Halogen, Isothiocyanate or Azide Substituted Compounds |
| 6,020,337 | Electronegative-Substituted Long Chain Xanthine Compounds |
| 5,795,897 | Oxohexyl Methylxanthine Compounds |
| 5,770,595 | Oxime Substituted Therapeutic Compounds |
| 5,929,081 | Method for Treating Diseases Mediated by Cellular Proliferation in Response |
| | to PDGF, EGF, FGF and VEGF |
| 5,859,018 | Method for Treating Diseases Mediated by Cellular Proliferation in Response |
| | to PDGF, EGF, FGF and VEGF |
| 5,795,898 | Method for Treating Diseases Mediated by Cellular Proliferation in Response |
| | to PDGF, EGF, FGF and VEGF |
| 6,100,271 | Therapeutic Compounds Containing Xanthinyl |
| 5,807,862 | Therapeutic Compounds |
| 6,043,250 | Methods for Using Therapeutic Compounds Containing Xanthinyl |
| 6,774,130 | Therapeutic Compounds for Inhibiting Interleukin-12 Signaling and Methods |
| | for Using Same |
| 6,878,715 | Therapeutic Compounds for Inhibiting Interleukin-12 Signaling and Methods |
| | for Using Same |
| 6,586,429 | Tricyclic Fused Xanthine Compounds and Their Uses (As Amended) |

Still further, additional BRM compounds or agents that may be for used in accordance with the principles of the present invention include, without limitation, members of the group consisting of the following cytokine formation blocking agents or methods: SiRNA (small interfering RNA); mTOR (mammalian target of Rapamycin); Leflunomide and active metabolites (e.g., A77 1726, LEF M); blockers of formation of advance glycation end products or small molecule or antibodies that inhibit the receptor for advance glycation end products (RAGE); Lipoxins or analogs thereof (e.g., LXA4); small molecule inhibitors of IL-12 (e.g., STA-S326, Synta Pharmaceuticals); monoclonal antibodies (e.g., anti-interleukin-12 monoclonal antibody (ABT-874, Abbott Laboratories); various methods for inhibiting cytokines described in Vanderbroeck, K., et al., “Inhibiting Cytokines of the Interleukin-12 Family: Recent Advances and Novel Challenges,” Journal of Pharmacy and Pharmacology, 56:145-160 (2004), and the like.

Isolating Islets

Methods of isolating pancreatic islet cells are known to those skilled in the art. See, e.g., Field et al., Transplantation 61:1554 (1996); Linetsky et al., Diabetes 46:1120 (1997). Fresh pancreatic tissue can be divided by mincing, teasing, comminution and/or collagenase digestion. The islets are then isolated from contaminating cells and materials by washing, filtering, centrifuging or picking procedures. Methods and apparatus for isolating and purifying islet cells are described in U.S. Pat. Nos. 5,447,863, 5,322,790, 5,273,904, and 4,868,121, the disclosures of which are entirely incorporated herein. The isolated pancreatic cells may optionally be cultured prior to encapsulation, using any suitable method of culturing islet cells as is known in the art. See, e.g., U.S. Pat. No. 5,821,121. Isolated cells may be cultured in a medium under conditions that helps to eliminate antigenic components (Transplant. Proc. 14:714-23 (1982)).

Encapsulation Materials and Techniques

Methods for formation of the encapsulating membranes are available in the art. See, e.g., U.S. Pat. Nos. 5,614,205 and 5,801,033, herein incorporated by reference. For example, membranes may be formed by conventional vacuum deposition and have a porosity which can be accurately controlled such that a selective membrane may be established. The composition of the semipermeable membrane used to encapsulate pancreatic islet cells is not critical provided that the membrane is cable of separating the insulin delivery source from the immune system of the host, while allowing glucose and other nutrients in and insulin to be secreted in response to ambient glucose levels. Examples of suitable materials suitable that may be used as the biocompatible semipermeable membranes include, without limitation, the following:
Alginate/poly (L-Lysine) capsules (AmCyte, Inc., MicroIslet, Inc., etc.)
Biodegradable capsules with only alginate
Hydrogel-based microcapsule using uncoated microsphere technology
Sodium alginate, cellulose sulfate, poly(methylene-co-quanidine), calcium chloride, and sodium chloride microcapsule (Taylor Wang, et al.)
Macro-Agarase Beads (The Rogosin Institute)
Polyethylene glycol capsules (“PEG”) (Neocrine Biosciences and Novocell)
Stealth microcapsules (Encell)
Islet Sheets (Islets Sheet Medical)
Planar membrane diffusion device (Theracyte, Baxter Healthcare) as described in Cell Transplantation (9:115-124, 2000)
Alginate-chitosan with PEG and crosslinkers such as carbondimide and glutaraldehyde
Collagen-containing alginate/poly (L-Lysine)/alginate microcapsules
Agaraose/polystyrene sulfonic acid microcapsules
Micro fabricated silicon based biocapsule

Microencapsulation of islet cells generally involves three steps: (a) generating microcapsules enclosing the islet cells (e.g., by forming droplets of cell-containing liquid alginate followed by exposure to a solution of calcium chloride to form a solid gel), (b) coating the resulting gelled spheres with additional outer coatings (e.g., outer coatings comprising polylysine) to form a semipermeable membrane; and (c) liquefying the original core get (e.g., by chelation using a solution of sodium citrate). The three steps are typically separated by washings in normal saline.

A preferred method of microencapsulating pancreatic cells is the alginate-polyamino acid technique. Briefly, islet cells are suspended in sodium alginate in saline, and droplets containing islets are produced. Droplets of cell-containing alginate flow into calcium chloride in saline. The negatively charged alginate droplets bind calcium and form a calcium alginate gel. The microcapsules are washed in saline and incubated with poly-L-lysine; the positively charged poly-1-lysine displaces calcium ions and binds (ionic) negatively charged alginate, producing an outer poly-electrolyte membrane. A final coating of sodium alginate may be added by washing the microcapsules with a solution of sodium alginate, which ionically bonds to the poly-L-lysine layer. See, e.g., U.S. Pat. No. 4,391,909 to Lim et al. This technique produces what has been termed a “single-wall” microcapsule. Preferred microcapsules are essentially round, small, and uniform in size. See, e.g., Wolters et al., J. Applied Biomaterials 3:281 (1992).

When desired, the alginate-polylysine microcapsules can then be incubated in sodium citrate to solubilize any calcium alginate that has not reacted with poly-1-lysine, i.e., to solubilize the internal core of sodium alginate containing the islet cells, thus producing a microcapsule with a liquefied cell-containing core portion. See, e.g., Lim and Sun, Science 210:908 (1980). Such microcapsules are referred to as having “chelated”, “hollow” or “liquid” cores.

A “double-wall” microcapsule is produced by following the same procedure as for single-wall microcapsules, but prior to any incubation with sodium citrate, the microcapsules are again incubated with poly-1-lysine and sodium alginate.

Alginates are linear polymers of mannuronic and guluronic acid residues. Monovalent cation alginate salts, e.g., Na-alginate, are generally soluble. Divalent cations such as Ca++, Ba++ or Sr++tend to interact with guluronate, providing crosslinking and forming stable alginate gels. Alginate encapsulation techniques typically take advantage of the gelling of alginate in the presence of divalent cation solutions. Alginate encapsulation of cells generally involves suspending the cells to be encapsulated in a solution of a monovalent cation alginate salt, generating droplets of this solution, and contacting the droplets with a solution of divalent cations. The divalent cations interact with the alginate at the phase transition between the droplet and the divalent cation solution, resulting in the formation of a stable alginate gel matrix being formed. A variation of this technique is reported in U.S. Pat. No. 5,738,876, wherein the cell is suffused with a solution of multivalent ions (e.g., divalent cations) and then suspended in a solution of gelling polymer (e.g., alginate), to provide a coating of the polymer.

Chelation of the alginate (degelling) core solubilizes the internal structural support of the capsule, may adversely affect the durability of the microcapsule, and is a harsh treatment of the encapsulated living cells. Degelling of the core may also cause leaching out of the unbound poly-lysine or solubilized alginate, resulting in a fibrotic reaction to the implanted microcapsule. The effect of core liquidation on glucose-stimulated insulin secretion by the encapsulated cells has been studied. See, e.g., Fritschy et al., Diabetes 40:37 (1991). The present inventors examined the function of islets enclosed in microcapsules that had not been subjected to liquefaction of the core (i.e., ‘solid’ or non-chelated microcapsules). It was found that culture of solid microcapsules prior to use enhanced the insulin response of the enclosed islets to glucose stimulation.

Alginate/polycation encapsulation procedures are simple and rapid, and represent a promising method for islet encapsulation for clinical treatment of diabetes. Many variations of this basic encapsulation method have been described in patents and the scientific literature. See, e.g., Chang et al., U.S. Pat. No. 5,084,350, which discloses microcapsules enclosed in a larger matrix, where the microcapsules are liquefied once the microcapsules are within the larger matrix. See, e.g., Tsang et al., U.S. Pat. No. 4,663,286, which discloses encapsulation using an alginate polymer, where the gel layer is cross-linked with a polycationic polymer such as polylysine, and a second layer formed using a second polycationic polymer (such as polyornithine); the second layer can then be coated by alginate. See, e.g., U.S. Pat. No. 5,762,959 to Soon-Shiong et al. which discloses a microcapsule having a solid (non-chelated) alginate gel core of a defined ratio of calcium/barium alginates, with polymer material in the core. U.S. Pat. Nos. 5,801,033 and 5,573,934 to Hubbell et al. describe alginate/polylysine microspheres having a final polymeric coating (e.g., polyethylene glycol (PEG)); Sawhney et al., Biomaterials 13:863 (1991) describe alginate/polylysine microcapsules incorporating a graft copolymer of poly-1-lysine and polyethylene oxide on the microcapsule surface, to improve biocompatibility; U.S. Pat. No. 5,380,536 describes microcapsules with an outermost layer of water soluble non-ionic polymers such as polyethylene(oxide). U.S. Pat. No. 5,227,298 to Weber et al. describes a method for providing a second alginate gel coating to cells already coated with polylysine alginate; both alginate coatings are stabilized with polylysine. U.S. Pat. No. 5,578,314 to Weber et al. provides a method for microencapsulation using multiple coatings of purified alginate. U.S. Pat. No. 5,693,514 to Dorian et al. reports the use of a non-fibrogenic alginate, where the outer surface of the alginate coating is reacted with alkaline earth metal cations comprising calcium ions and/or magnesium ions, to form an alkaline earth metal alginate coating. The outer surface of the alginate coating is not reacted with polylysine. U.S. Pat. No. 5,846,530 to Soon-Shiong describes microcapsules containing cells that have been individually coated with polymerizable alginate, or polymerizable polycations such as polylysine, prior to encapsulation.

Microcapsules

The methods of the present invention are may be used with any microcapsule that contains living cells secreting a desirable biological substance (preferably pancreatic cells and more preferably islet cells), where the microcapsule comprises an inner gel or liquid core containing the cells of interest, or a liquid core containing the cells of interest, bounded by a semi-permeable membrane surrounding the cell-containing core. The inner core is preferably composed of a water-soluble gelling agent; preferably the water-soluble gelling agent comprises plural groups that can be ionized to form anionic or cationic groups. The presence of such groups in the gel allows the surface of the gel bead to be cross-linked to produce a membrane, when exposed to polymers containing multiple functionalities having a charge opposite to that of the gel.

Cells suspended in a gellable medium (such as alginate) may be formed into droplets using any suitable method as is known in the art, including but not limited to emulsification (see e.g., U.S. Pat. No. 4,352,883), extrusion from a needle (see, e.g., U.S. Pat. No. 4,407,957; Nigam et al., Biotechnology Techniques 2:271-276 (1988)), use of a spray nozzle (Plunkett et al., Laboratory Investigation 62:510-517 (1990)), or use of a needle and pulsed electrical electrostatic voltage (see, e.g., U.S. Pat. No. 4,789,550; U.S. Pat. No. 5,656,468).

The water-soluble gelling agent is preferably a polysaccharide gum, and more preferably a polyanionic polymer. An exemplary water-soluble gelling agent is an alkali metal alginate such as sodium alginate. The gelling agent preferably has free acid functional groups and the semi-permeable membrane is formed by contacting the gel with a polymer having free amino functional groups with cationic charge, to form permanent crosslinks between the free amino acids of the polymer and the acid functional groups. Preferred polymers include polylysine, polyethylenimine, and polyarginine. A particularly preferred microcapsule contains cells immobilized in a core of alginate with a poly-lysine coating; such microcapsules may comprise an additional external alginate layer to form a multi-layer alginate-polylysine-alginate microcapsule. See U.S. Pat. No. 4,391,909 to Lim et al, the contents of which are incorporated by reference herein in their entirety. See also U.S. Pat. No. 6,352,707 to Usala.

When desired, liquefaction of the core gel may be carried out by any suitable method as is known in the art, such as ion exchange or chelation of calcium ion by sodium citrate or EDTA.

Microcapsules useful in the present invention may have at least one semipermeable surface membrane surrounding a cell-containing core. The surface membrane permits the diffusion of nutrients, biologically active molecules and other selected products through the surface membrane and into the microcapsule core. The surface membrane contains pores of a size that determines the molecular weight cut-off of the membrane. Where the microcapsule contains insulin-secreting cells, the membrane pore size is chosen to allow the passage of insulin from the core to the external environment, but to exclude the entry of host immune response factors.

As used herein, a “poly-amino acid-alginate microsphere” refers to a capsule of less than 2 mm in diameter having an inner core of cell-containing alginate bounded by a semipermeable membrane formed by alginate and poly-1-lysine. Viable cells encapsulated using an anionic polymer such as alginate to provide a gel layer, where the gel layer is subsequently cross-linked with a polycationic polymer (e.g., an amino acid polymer such as polylysine0. See e.g., U.S. Pat. Nos. 4,806,355, 4,689,293 and 4,673,566 to Goosen et al.; U.S. Pat. Nos. 4,409,331, 4,407,957, 4,391,909 and 4,352,883 to Lim et al.; U.S. Pat. Nos. 4,749,620 and 4,744,933 to Rha et al.; and U.S. Pat. No. 5,427,935 to Wang et al. Amino acid polymers that may be used to encapsulate islet cells in alginate include the cationic amino acid polymers of lysine, arginine, and mixtures thereof.

A method of encapsulating a core material within a semi-permeable membrane (e.g., a hydrogel) comprises: (a) placing the material in an aqueous solution of a water-soluble polymeric substance that can be reversibly gelled and which has free acid groups, (b) forming the solution into droplets, (c) gelling the droplets to produce discrete shape-retaining temporary capsules, (d) forming biocompatible semi-permeable membranes about the temporary capsules by contact between the temporary capsules and a biocompatible polyamino acid polymer containing free amino groups to cause ionic reaction with the acid groups in a surface layer of the capsule to provide a positively-charged surface, and (e) contacting said microcapsules formed in step (d) with a non-toxic biocompatible water soluble polymeric material which contains free negatively-charged groups capable of ionic reaction with the free amino groups of said polyamino acid polymer in surface layer of the microcapsule, thereby to form an outer coating of said biocompatible polymeric material on said microcapsules having a negatively-charged surface, said semi-permeable membrane formation and said contact thereof with biocompatible polymeric material being such as to form microcapsules having a diameter of about 50 to about 2000 μm and a semi-permeable membrane thickness of about 5 to about 20 μm, and being such as to produce microcapsules capable of resisting degradation and remaining permeable in vivo.

Culture of Isolated Cells

Generally, pancreatic islets are isolated by collagenase digestion of pancreatic tissue. This process involves subjecting the islet cells to a period of hypoxia which is then followed by reoxygenation. Hypoxia-reoxygenation produces an injury that is linked to excessive production of oxygen free radicals which impair the function, and cause the death, of islet cells, particularly those isolated from the pancreas of large mammals such as pigs and humans.

Where culture of isolated islets or islet cells prior to microencapsulation is beneficial, the islets are cultured according to known cell culture techniques for a period of at least 3 hours, more preferably from 12-36 hours, and more preferably from 18-24 hours, in a culture medium containing a BRM or a combination of a BRM and an antioxidant, an anti-endotoxin, and an antibiotic.

Culture of isolated pancreatic islets or any insulin producing cells (human or xeno) to improve glucose-stimulated insulin secretion may utilize any suitable anti-oxidant as is known in the art. As used herein, an antioxidant is a compound that neutralizes free radicals or prevents or suppresses the formation of free radicals. Particularly preferred are molecules including thiol groups such as reduced glutathione (GSH) or its precursors, glutathione or glutathione analogs, glutathione monoester, and N-acetylcysteine. Other suitable anti-oxidants include superoxide dismutase, catalase, vitamin E, Trolox, lipoic acid, lazaroids, butylated hydroxyanisole (BHA), vitamin K, and the like. Glutathione, for example, may be used in a concentration range of from about 2 to about 10 mM. See, e.g., U.S. Pat. Nos. 5,710,172; 5,696,109; 5,670,545.

Culture of isolated pancreas cells to improve glucose-stimulated insulin secretion may utilize any suitable antibiotic as is known in the art. Suitable antibiotics include penicillins, tetracyclines, cephalosporins, macrolides, .beta.-lactams and aminoglycosides; examples of such suitable antibiotics include streptomycin and amphotericin B.

Culture of isolated pancreas cells to improve glucose-stimulated insulin secretion may utilize any suitable anti-endotoxin as is known in the art. Endotoxins are bacterial toxins, complex phospholipid-polysaccharide molecules that form a part of the cell wall of a variety of Gram-negative bacteria. Anti-endotoxins are compounds that destroy or inhibit the activity of endotoxins. Endotoxins are intracellular toxins, and are complex phospholipid-polysaccharide macromolecules that form a part of the cell wall of a variety of Gram-negative bacteria, including enterobacteria, vibrios, brucellae and neisseriae. Suitable anti-endotoxins for use in culturing islet cells include L-N.sup.G-Monomethylarginine (L-NMMA, 2 mM), lactoferrin (100 .mu.g/ml), N-acetylcysteine (NAC, 1 mM), adenosine receptor antagonists such as bamiphylline (theophylline) and anti-lipopolysaccharide compounds such as echinomycine (10 nM), and the like.

Cryopreservation of Cells

Mammalian tissue remains viable in vitro only for short periods of time, usually days. Loss of islet cells suitable for transplantation may be avoided by viable cryopreservation and cold storage of the cells. Microencapsulated islet cells respond poorly to cryopreservation. However, cryopreservation of naked (unencapsulated) islet cells did not adversely affect their later function in microcapsules when the cells were first cryopreserved, then thawed and microencapsulated. Frozen and thawed microencapsulated islets responded poorly to glucose stimulation; in comparison, ‘naked’ islet cells that were cryopreserved and then thawed retained their ability to respond to glucose stimulation and were suitable for microencapsulation. Islet cells can thus be preserved by cryopreservation, thawed and microencapsulated just prior to use.

Methods of cryopreservation are well known in the art. In general terms, cryopreservation of animal cells involves freezing the cells in a mixture of a growth medium and another liquid that prevents water from forming ice crystals, and then storing the cells at liquid nitrogen temperatures (e.g., from about −80 to about −196° C.).

An aspect of the present invention is the cryopreservation of isolated mammalian cells in a cryopreservation medium containing an antioxidant, followed by microencapsulation of the cells prior to in vivo implantation. A preferred embodiment of the present invention is the cryopreservation of isolated islets or islet cells in a cryopreservation medium containing an antioxidant as described herein, followed by microencapsulation prior to in vivo implantation.

More preferably, the cells are cryopreserved in a medium containing at least one of, or a combination of, the following: a BRM, an antioxidant, an antiendotoxin, and an antibiotic (each as described above). Preferably, the cells are cryopreserved in a medium containing at least one each of an antioxidant, a BRM, an anti-endotoxin, and an antibiotic (each as described above).

Culture of Microspheres.

Culturing microcapsules prior to use enhances subsequent glucose-stimulated insulin production to results in islets that respond better to a glucose challenge than islets contained in fresh (non-cultured) microcapsules.

Culture of microencapsulated cells is carried out in a manner similar to the culture of isolated cells, as described herein and as generally known in the art. Accordingly, a method of the present invention is the culture of microcapsules (with either solid or liquid cores containing living cells) prior to implantation in vivo, to enhance or improve the ability of the microcapsule to produce a desired cell secretory product. A particularly preferred embodiment is the culture, prior to implantation, of gelled or solid-core alginate-polylysine microcapsules containing pancreatic islets or islet cells. Microcapsules are cultured for a period of at least 3 hours, more preferably from 12 to 36 hours, or 18 to 24 hours, prior to implantation.

Preferably the microcapsules are cultured in a medium containing at least one BRM, or a combination of, the following: a BRM, an antibiotic, an antioxidant, and an antiendotoxin (as described above). More preferably, the microcapsules are cultured in a medium containing at least one each of an antioxidant, a beta-cell growth, differentiating or neogenesis factor, a BRM, an anti-endotoxin, and an antibiotic (each as described above).

Transplantation

Encapsulated islet cells or any insulin producing cells that are prepared according to the present invention may be transplanted into subjects as a treatment for insulin-dependent diabetes; such transplantation may be into the peritoneal cavity of the subject. An amount of encapsulated islet cells to produce sufficient insulin to control glycemia in the subject is provided by any suitable means, including but not limited to surgical implantation and intraperitoneal injection. Preferably, transplants of at least 6,000 islets, equivalent to 150 μm in size, per kilogram of recipient body weight, to achieve euglycemia. However, it will be apparent to those skilled in the art that the quantity of microcapsules transplanted depends on the ability of the microcapsules to provide insulin in vivo, in response to glucose stimulation. One skilled in the art will be able to determine suitable transplantation quantities of microcapsules, using techniques as are known in the art.

In sum, without limiting the foregoing, the present invention is directed a composition for treating diabetes (Types 1 or 2, and LADA), said composition having at least the following features: (1) a delivery vehicle comprising a selectively permeable membrane that allows passage of glucose, insulin and other nutrients through the membrane, but prevents large molecules such as antibodies or inflammatory cells from passing through the membrane; (2) a population of islet cells or insulin producing cells encapsulated by said membrane; and (3) a BRM, said BRM being in contact with the membrane or being encapsulated by the membrane. The BRM may be linked to a surface of the membrane (e.g., via covalent bonds, ionic bonds, hydrogen bonds, aromatic bonds, metallic bonds, hydrophobic/hydrophilic interactions, etc.). The membrane may comprise a polymer matrix and the BRM may be embedded in the polymer matrix of the delivery vehicle. The present invention is also directed to a composition of matter having at least the following features: (1) a selectively permeable membrane that allows passage of glucose, insulin and other nutrients through the membrane, but prevents large molecules such as antibodies or inflammatory cells from passing through the membrane; and (2) a BRM linked to the membrane (e.g., via covalent bonds, ionic bonds, hydrogen bonds, aromatic bonds, metallic bonds, hydrophobic/hydrophilic interactions, etc.). The composition may be cultured in a medium containing at least one (1) an antioxidant, (2) a beta-cell growth, differentiating or neogenesis factor, (3) an anti-endotoxin, and (4) an antibiotic (each as described above).

Although illustrative embodiments have been described in detail, it is to be understood that the invention is not limited to those precise embodiments, and that various changes and modifications can be effected therein by one skilled in the art without departing from the scope and spirit of the invention.

1. An encapsulated material for treating diabetes comprising:
(a) a selectively permeable membrane;
(b) a population of islet cells or insulin producing cells encapsulated by said membrane; and
(c) a biological response modifier.

2. The encapsulated material of claim 1, wherein the membrane allows passage of glucose, insulin and other nutrients through the membrane, selectively.

3. The encapsulated material of claim 1, wherein the biological response modifier is bonded to the membrane.

4. The encapsulated material of claim 1, wherein the membrane is a polymeric matrix.

5. The encapsulated material of claim 1, further comprising at least one member of the group consisting of an antioxidant, a beta-cell growth factor, a beta-cell differentiating factor, a beta-cell neogenesis factor, an anti-endotoxin, and an antibiotic.

6. The encapsulated material of claim 1, wherein the biological response modifier is a compound, including resolved enantiomers, diastereomers, tautomers, salts and solvates thereof, having the following formula: 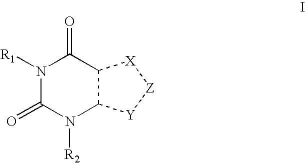
wherein:
X, Y and Z are independently selected from a member of the group consisting of C(R₃), N, N(R₃) and S;
R₁ is selected from a member of the group consisting of hydrogen, methyl, C₍₅₋₉₎alkyl, C₍₅₋₉₎alkenyl, C₍₅₋₉₎alkynyl, C₍₅₋₉₎hydroxyalkyl, C₍₃₋₈₎alkoxyl, C₍₅₋₉₎alkoxyalkyl, the R₁ being optionally substituted;
R₂ and R₃ are independently selected from a member of the group consisting of hydrogen, halo, oxo, C₍₁₋₂₀₎alkyl, C₍₁₋₂₀₎hydroxyalkyl, C₍₁₋₂₀₎thioalkyl, C₍₁₋₂₀₎alkylamino, C₍₁₋₂₀₎alkylaminoalkyl, C₍₁₋₂₀₎aminoalkyl, C₍₁₋₂₀₎aminoalkoxyalkenyl, C₍₁₋₂₀₎aminoalkoxyalkynyl, C₍₁₋₂₀₎diaminoalkyl, C₍₁₋₂₀₎triaminoalkyl, C₍₁₋₂₀₎tetraaminoalkyl, C₍₅₋₁₅₎aminotrialkoxyamino, C₍₁₋₂₀₎alkylamido, C₍₁₋₂₀₎alkylamidoalkyl, C₍₁₋₂₀₎amidoalkyl, C₍₁₋₂₀₎acetamidoalkyl, C₍₁₋₂₀₎alkenyl, C₍₁₋₂₀₎alkynyl, C₍₃₋₈₎alkoxyl, C₍₁₋₁₁₎alkoxyalkyl, and C₍₁₋₂₀₎dialkoxyalkyl.

7. The encapsulated material of claim 6, wherein R₁ is substituted with a member of the group consisting of N—OH, acylamino, cyano group, sulfo, sulfonyl, sulfinyl, sulfhydryl (mercapto), sulfeno, sulfanilyl, sulfamyl, sulfamino, and phosphino, phosphinyl, phospho, phosphono and —NR^{a}R^{b}, wherein each of R^{a} and R^{b} may be the same or different and each is selected from the group consisting of hydrogen, optionally substituted alkyl, cycloalkyl, alkenyl, cycloalkenyl, alkynyl, aryl, heteroaryl and heterocyclic group.

8. The encapsulated material of claim 6, wherein R₂ and R₃ are selected from the group consisting of methyl, ethyl, oxo, isopropyl, n-propyl, isobutyl, n-butyl, t-butyl, 2-hydroxyethyl, 3-hydroxypropyl, 3-hydroxy-n-butyl, 2methoxyethyl, 4-methoxy-n-butyl, 5-hydroxyhexyl, 2-bromopropyl, 3-dimethylaminobutyl, 4-chloropentyl, methylamino, aminomethyl, and methylphenyl.

9. The encapsulated material of claim 6, wherein each R₂ and R₃ is substituted with one or more members of the group consisting of hydroxyl, methyl, carboxyl, furyl, furfuryl, biotinyl, phenyl, naphthyl, amino group, amido group, carbamoyl group, cyano group, sulfo, sulfonyl, sulfinyl, sulfhydryl, sulfeno, sulfanilyl, sulfamyl, sulfamino, phosphino, phosphinyl, phospho, phosphono, N—OH, —Si(CH₃)₃, C₍₁₋₃₎alkyl, C₍₁₋₃₎hydroxyalkyl, C₍₁₋₃₎thioalkyl, C₍₁₋₃₎alkylamino, benzyldihydrocinnamoyl group, benzoyldihydrocinnamido group, optionally substituted heterocyclic group and optionally substituted carbocyclic group.

10. The encapsulated material of claim 6, wherein the heterocyclic group or carbocyclic group is substituted with one or more members of the group consisting of halo, hydroxyl, nitro, SO₂NH₂, C₍₁₋₆₎alkyl, C₍₁₋₆₎haloalkyl, C₍₁₋₈₎alkoxyl, C₍₁₋₁₁₎alkoxyalkyl, C₍₁₋₆₎alkylamino, and C₍₁₋₆₎aminoalkyl.

11. The encapsulated material of claim 10, wherein the heterocyclic group is a member selected from the group consisting of acridinyl, aziridinyl, azocinyl, azepinyl, benzimidazolyl, benzodioxolanyl, benzofuranyl, benzothiophenyl, carbazole, 4a H carbazole, chromanyl, chromenyl, cinnolinyl, decahydroquinolinyl, dioxoindolyl, furazanyl, furyl, furfuryl, imidazolidinyl, imidazolinyl, imidazolyl, 1H indazolyl, indolenyl, indolinyl, indolizinyl, indolyl, 3H indolyl, isobenzofuranyl, isochromanyl, isoindolinyl, isoindolyl, isoquinolinyl, isothiazolyl, isoxazolyl, morpholinyl, naphthalenyl, naphthyridinyl, norbornanyl, norpinanyl, octahydroisoquinolinyl, oxazolidinyl, oxazolyl, oxiranyl, perimidinyl, phenanthridinyl, phenanthrolinyl, phenarsazinyl, phenazinyl, phenothiazinyl, phenoxathiinyl, phenoxazinyl, phenyl, phthalazinyl, piperazinyl, piperidinyl, 4 piperidonyl, piperidyl, pteridinyl, purinyl, pyranyl, pyrazinyl, pyrazolidinyl, pyrazolinyl, pyrazolyl, pyrenyl, pyridazinyl, pyridinyl, pyridyl, pyridyl, pyrimidinyl, pyrrolidinyl, 2 pyrrolidonyl, pyrrolonyl, pyrrolyl, 2H pyrrolyl, quinazolinyl, 4H quinolizinyl, quinolinyl, quinoxalinyl, quinuclidinyl, β carbolinyl, tetrahydrofuranyl, tetrahydroisoquinolinyl, tetrahydroquinolinyl, tetrazolyl, 6H 1,2,5 thiadiazinyl, 2H,6H 1,5,2 dithiazinyl, thianthrenyl, thiazolyl, thienyl, thiophenyl, triazinyl, xanthenyl and xanthinyl.

12. The encapsulated material of claim 11, wherein the carbocyclic group is a member selected from the group consisting of adamantyl, anthracenyl, benzamidyl, benzyl, bicyclo[2.2.1]heptanyl, bicyclo[2.2.1]hexanyl, bicyclo[2.2.2]octanyl, bicyclo[3.2.0]heptanyl, bicyclo[4.3.0]nonanyl, bicyclo[4.4.0]decanyl, biphenyl, biscyclooctyl, cyclobutyl, cyclobutenyl, cycloheptyl, cycloheptenyl, cyclohexanedionyl, cyclohexenyl, cyclohexyl, cyclooctanyl, cyclopentadienyl, cyclopentanedionyl, cyclopentenyl, cyclopentyl, cyclopropyl, decalinyl, 1,2-diphenylethanyl, indanyl, 1-indanonyl, indenyl, naphthyl, napthlalenyl, phenyl, resorcinolyl, stilbenyl, tetrahydronaphthyl, tetralinyl, tetralonyl, and tricyclododecanyl.

13. The encapsulated material of claim 1, wherein the biological response modifier is 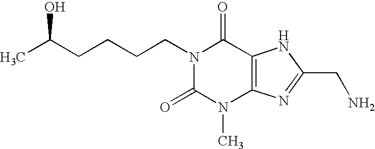
or a pharmaceutically acceptable salt thereof.

14. The pharmaceutical composition of claim 1, wherein the biological response modifier is 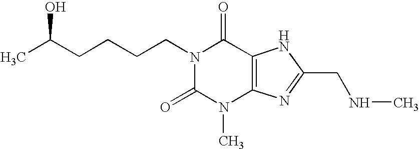
or a pharmaceutically acceptable salt thereof.

15. The pharmaceutical composition of claim 1, or pharmaceutically acceptable salt thereof, wherein the biological response modifier is a member selected from the group consisting of:

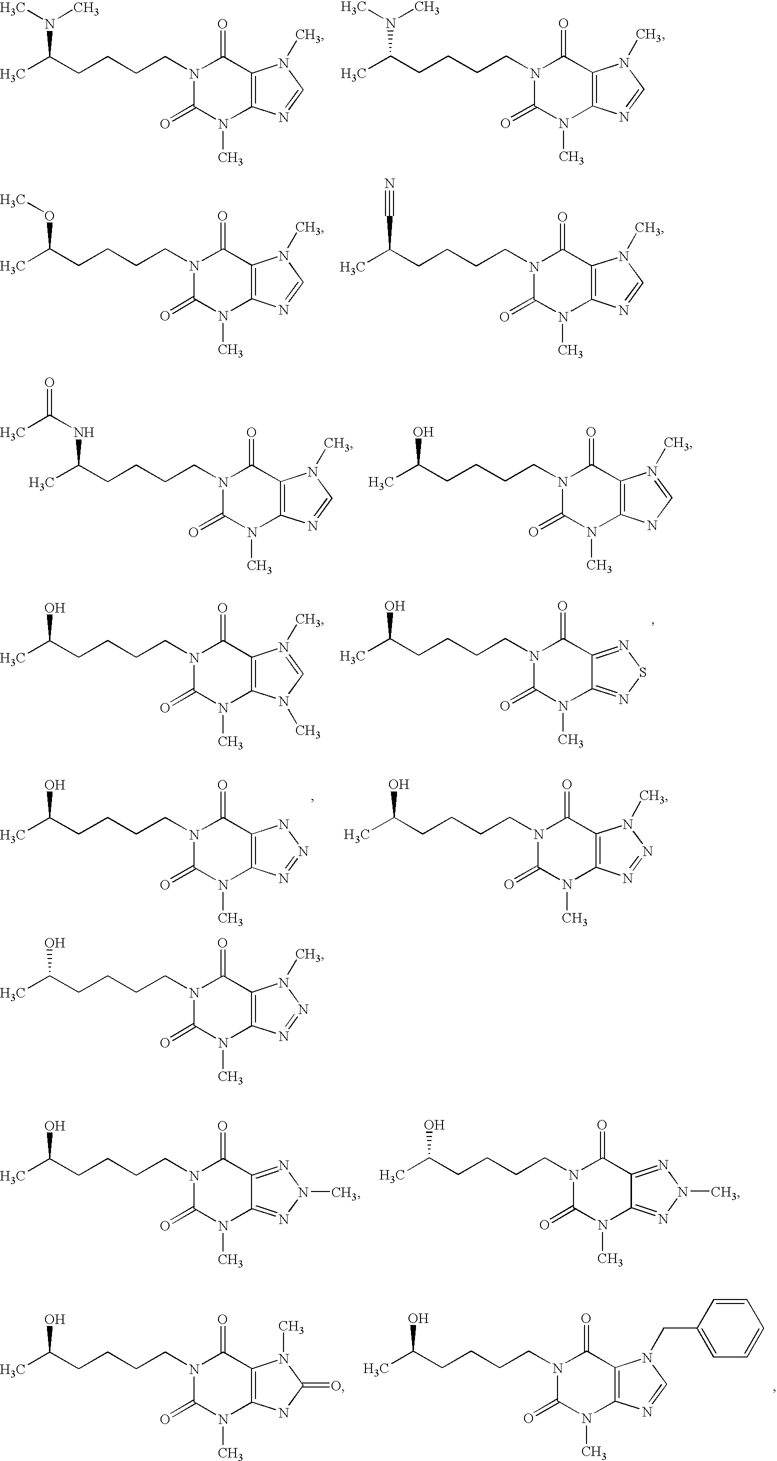

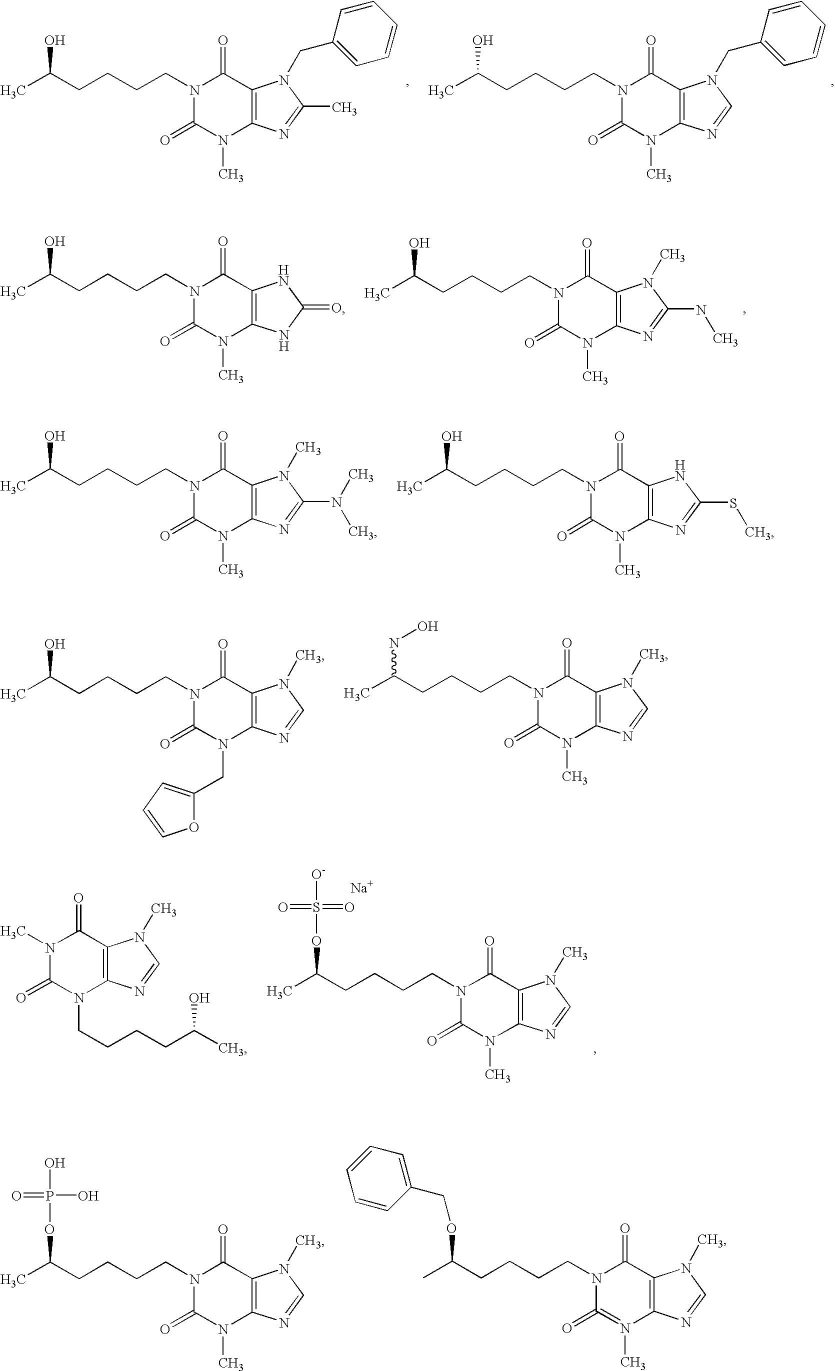

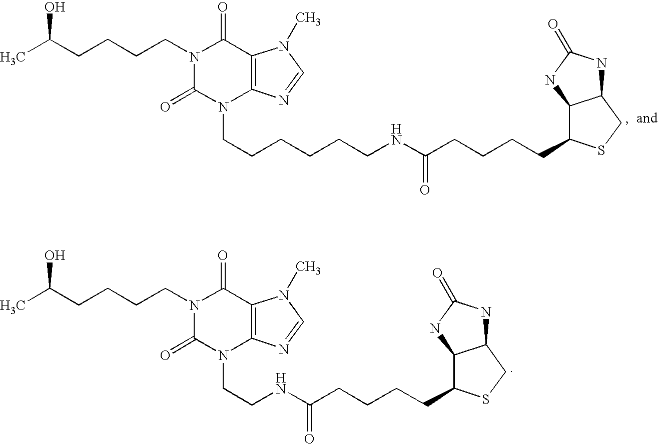

16. The pharmaceutical composition of claim 1, wherein the biological response modifier is selected from the group consisting of the compounds defined in Table 1.

17. The pharmaceutical composition of claim 5, wherein the β-cell growth or differentiating factor comprises a GLP-1 analog selected from the group consisting of: Exendin-4 (Ex-4); Exenatide; Liraglutide; CJC-1131; Albugon; and LY-548806.

18. The pharmaceutical composition of claim 5, wherein the β-cell growth or differentiating factor is a DPP-IV inhibitor selected from the group consisting of: Sitagliptin, Vildagliptin, NVP DPP728, Saxagliptin, P32/98, FE 999011, and PHX1149.

19. The pharmaceutical composition of claim 5, wherein the β-cell growth or differentiating factor is a member selected from the group consisting of: gastric inhibitory polypeptide (GIP) and analogs thereof; gastrin; epidermal growth factor 1; islet neogenesis therapy; insulin like growth factor 1 or 2; Parathyroid hormone related peptide (PTHrP); Hepatocyte growth factor; islet neogenesis associated protein (INGAP); NVP-LAQ824; TrichostatinA-0; hydroxamate; suberanihohydroxamic; tetrapeptides, apicidin; trapoxin; CG1521; scriptide; oxamflatin; pyroxamide; propenamides; chlamydocin; diheteropeptin; WF-3136; Cyl-1; Cyl-2; FR 901228; cyclic-hydroxamic-acid-containing peptides; MS-275, CI-994; depudecin, Neurogen 3, PDX-1, NKX6.1, glucagon-like peptide 1 fragments, glucagon-like peptide 1(7-36)amide and glucagon-like peptide 1(7-37).

20. A method for restoring β-cell mass and function in a mammal, comprising administering to the mammal a therapeutically effective amount of the encapsulated material of claim 1.

21. A method for treating diabetes in a mammal, the method comprising transplanting to the mammal a therapeutically effective amount of the encapsulated material of claim 1.

22. A means for delivering a therapeutically effective amount of the encapsulated material of claim 1 to a mammal.
